(19) United States
(12) Patent Application Publication
(10) Pub. No.: US 2006/0115813 A1
(43) Pub. Date: Jun. 1, 2006

(54) Intracellular signaling molecules

(75) Inventors: Junming Yang, SAN JOSE, CA (US); Brooke M. Emerling, Chicago, IL (US); Y Tom Tang, San Jose, CA (US); Mariah R. Baugh, Los Angeles, CA (US); Ernestine A. Lee, Kensington, CA (US); Jayalaxmi Ramkumar, Fremont, CA (US); Henry Yue, Sunnyvale, CA (US); Jennifer A. Griffin, Fremont, CA (US); Narinder K. Chawla, Union City, CA (US); Bao Tran, Santa clara, CA (US); Danniel B. Nguyen, San Jose, CA (US); Farrah A. Khan, Des Plaines, IL (US); Ameena R. Gandhi, San Francisco, CA (US); April J A Hafalia, Daly City, CA (US); Anita Swarnakar, San Francisco, CA (US); Rajagopal Gururajan, San Jose, CA (US); Jennifer L Policky, San Jose, CA (US); Monique G. Yao, Mountain view, CA (US); Bridget A. Warren, San Marcos, CA (US); Kimberly J. Gietzen, San Jose, CA (US); Vicki S. Elliott, San Jose, CA (US); Soo Yeun Lee, Mountain view, CA (US); Bharati Sanjanwala, Los Altos, CA (US); Cynthia qD. Honchell, San Francisco, CA (US); Ian J. Forsythe, Edmonton (CA); Ann E. Gorvad, Bellingham, WA (US); Thomas W. Richardson, Redwood City, CA (US); Sally Lee, San Jose, CA (US); Ines Barroso, Cambridge (GB)

Correspondence Address: INCYTE CORPORATION;EXPERIMENTAL STATION, ROUTE 141 & HENRY CLAY ROAD, BLDG. E336, WILMINGTON, DE, 19880, US

(73) Assignee: INCYTE CORPORATION, PALO ALTO, CA (US)

(21) Appl. No.: 10/480,962

(22) PCT Filed: Jun. 6, 2002
(86) PCT No.: PCT/US02/17955

§ 371 Date: Sep. 9, 2004

Related U.S. Application Data
(60) Provisional application No. 60/297,010, filed on Jun. 8, 2001.
(60) Provisional application No. 60/298,706, filed on Jun. 15, 2001.
(60) Provisional application No. 60/299,998, filed on Jun. 20, 2001.
(60) Provisional application No. 60/300,377, filed on Jun. 21, 2001.
(60) Provisional application No. 60/301,871, filed on Jun. 29, 2001.
(60) Provisional application No. 60/303,349, filed on Jul. 5, 2001.
(60) Provisional application No. 60/303,403, filed on Jul. 6, 2001.
(60) Provisional application No. 60/351,927, filed on Jan. 25, 2002.

(51) Int. Cl. C12Q 1/68; A61K 38/17; C07K 14/47; C07H 21/04; C12P 21/06

(52) U.S. Cl. 435/6; 435/69.1; 435/320.1; 435/325; 530/350; 514/12; 536/23.5

(57) ABSTRACT

Various embodiments of the invention provide human intracellular signaling molecules (INTSIG) and polynucleotides which identify and encode INTSIG. Embodiments of the invention also provide expression vectors, host cells, antibodies, agonists, and antagonists. Other embodiments provide methods for diagnosing, treating, or preventing disorders associated with aberrant expression of INTSIG.

### TECHNICAL FIELD

The invention relates to novel nucleic acids, intracellular signaling molecules encoded by these nucleic acids, and to the use of these nucleic acids and proteins in the diagnosis, treatment, and prevention of cell proliferative, autoimmune/inflammatory, neurological, gastrointestinal, reproductive, developmental, and vesicle trafficking disorders. The invention also relates to the assessment of the effects of exogenous compounds on the expression of nucleic acids and intracellular signaling molecules.

### BACKGROUND OF THE INVENTION

Cell-cell communication is essential for the growth, development, and survival of multicellular organisms. Cells communicate by sending and receiving molecular signals. An example of a molecular signal is a growth factor, which binds and activates a specific transmembrane receptor on the surface of a target cell. The activated receptor transduces the signal intracellularly, thus initiating a cascade of biochemical reactions that ultimately affect gene transcription and cell cycle progression in the target cell.

Intracellular signaling is the process by which cells respond to extracellular signals (hormones, neurotransmitters, growth and differentiation factors, etc.) through a cascade of biochemical reactions that begins with the binding of a signaling molecule to a cell membrane receptor and ends with the activation of an intracellular target molecule. Intermediate steps in the process involve the activation of various cytoplasmic proteins by phosphorylation via protein inases, and their deactivation by protein phosphatases, and the eventual translocation of some of these activated proteins to the cell nucleus where the transcription of specific genes is triggered. The intracellular signaling process regulates all types of cell functions including cell proliferation, cell differentiation, and gene transcription, and involves a diversity of molecules including protein kinases and phosphatases, and second messenger molecules such as cyclic nucleotides, calcium-calmodulin, inositol, and various mitogens that regulate protein phosphorylation.

A distinctive class of signal transduction molecules are involved in odorant detection. The process of odorant detection involves specific recognition by odorant receptors. The olfactory mucosa also appears to possess an additional group of odorant-binding proteins which recognize and bind separate classes of odorants. For example, cDNA clones from rat have been isolated which correspond to mRNAs highly expressed in olfactory mucosa but not detected in other tissues. The proteins encoded by these clones are homologous to proteins that bind lipopolysaccharides or polychlorinated biphenyls, and the different proteins appear to be expressed in specific areas of the mucosal tissue. These proteins are believed to interact with odorants before or after specific recognition by odorant receptors, perhaps acting as selective signal filters (Dear, T. N. et al. (1991) EMBO J. 10:2813-2819; Vogt, R. G. et al. (1991) J. Neurobiol. 22:74-84).

Cells also respond to changing conditions by switching off signals. Many signal transduction proteins are short-lived and rapidly targeted for degradation by covalent ligation to ubiquitin, a highly conserved small protein. Cells also maintain mechanisms to monitor changes in the concentration of denatured or unfolded proteins in membrane-bound extracytoplasmic compartments, including a transmembrane receptor that monitors the concentration of available chaperone molecules in the endoplasmic reticulum and transmits a signal to the cytosol to activate the transcription of nuclear genes encoding chaperones in the endoplasmic reticulum.

Certain proteins in intracellular signaling pathways serve to link or cluster other proteins involved in the signaling cascade. These proteins are referred to as scaffold, anchoring, or adaptor proteins. (For review, see Pawson, T. and J. D. Scott (1997) Science 278:2075-2080.) As many intracellular signaling proteins such as protein kinases and phosphatases have relatively broad substrate specificities, the adaptors help to organize the component signaling proteins into specific biochemical pathways. Many of the above signaling molecules are characterized by the presence of particular domains that promote protein-protein interactions. A sampling of these domains is discussed below, along with other important intracellular messengers.

Intracellular Signaling Second Messenger Molecules

Protein Phosphorylation

Protein kinases and phosphatases play a key role in the intracellular signaling process by controlling the phosphorylation and activation of various signaling proteins. The high energy phosphate for this reaction is generally transferred from the adenosine triphosphate molecule (ATP) to a particular protein by a protein kinase and removed from that protein by a protein phosphatase. Protein kinases are roughly divided into two groups: those that phosphorylate serine or threonine residues (serine/threonine kinases, STK) and those that phosphorylate tyrosine residues (protein tyrosine kinases, PTK). A few protein kinases have dual specificity for serine/threonine and tyrosine residues. Almost all kinases contain a conserved 250-300 amino acid catalytic domain containing specific residues and sequence motifs characteristic of the kinase family (Hardie, G. and S. Hanks (1995) The Protein Kinase Facts Books, Vol 1:7-20, Academic Press, San Diego, Calif.).

STKs include the second messenger dependent protein kinases such as the cyclic-AMP dependent protein kinases (PKA), involved in mediating hormone-induced cellular responses; calcium-calmodulin (CaM) dependent protein kinases, involved in regulation of smooth muscle contraction, glycogen breakdown, and neurotransmission; and the mitogen-activated protein kinases (MAP kinases) which mediate signal transduction from the cell surface to the nucleus via phosphorylation cascades. Altered PKA expression is implicated in a variety of disorders and diseases including cancer, thyroid disorders, diabetes, atherosclerosis, and cardiovascular disease (Isselbacher, K. J. et al. (1994) Harrison's Pinci les of Internal Medicine, McGraw-Hill, New York, N.Y., pp. 416-431, 1887).

PTKs are divided into transmembrane, receptor PTKs and nontransmembrane, non-receptor PTKs. Transmembrane PTKs are receptors for most growth factors. Non-receptor PTKs lack transmembrane regions and, instead, form complexes with the intracellular regions of cell surface receptors. Receptors that function through non-receptor PTKs include those for cytokines and hormones (growth hormone and prolactin) and antigen-specific receptors on T and B lymphocytes. Many of these PTKs were first identified as the products of mutant oncogenes in cancer cells in which their activation was no longer subject to normal cellular controls. In fact, about one third of the known oncogenes encode PTKs, and it is well known that cellular transformation (oncogenesis) is often accompanied by increased tyrosine phosphorylation activity (Charbonneau H and N. K. Tonks (1992) Annu. Rev. Cell Biol. 8:463-493).

An additional family of protein kinases previously thought to exist only in prokaryotes is the histidine protein kinase family (HPK). HPKs bear little homology with mammalian STKs or PTKs but have distinctive sequence motifs of their own (Davie, J. R. et al. (1995) J. Biol. Chem. 270:19861-19867). A histidine residue in the N-terminal half of the molecule (region I) is an autophosphorylation site. Three additional motifs located in the C-terminal half of the molecule include an invariant asparagine residue in region II and two glycine-rich loops characteristic of nucleotide binding domains in regions III and IV. Recently a branched chain alpha-ketoacid dehydrogenase kinase has been found with characteristics of HPK in rat (Davie et al., supra).

Protein phosphatases regulate the effects of protein kinases by removing phosphate groups from molecules previously activated by kinases. The two principal categories of protein phosphatases are the protein (serine/threonine) phosphatases (PPs) and the protein tyrosine phosphatases (PTPs). PPs dephosphorylate phosphoserine/threonine residues and are important regulators of many cAMP-mediated hormone responses (Cohen, P. (1989) Annu. Rev. Biochem. 58:453-508). PIPs reverse the effects of protein tyrosine kinases and play a significant role in cell cycle and cell signaling processes (Charbonneau and Tonks, supra). As previously noted, many PTKs are encoded by oncogenes, and oncogenesis is often accompanied by increased tyrosine phosphorylation activity. It is therefore possible that PTPs may prevent or reverse cell transformation and the growth of various cancers by controlling the levels of tyrosine phosphorylation in cells. This hypothesis is supported by studies showing that overexpression of PTPs can suppress transformation in cells, and that specific inhibition of PTPs can enhance cell transformation (Charbonneau and Totks, supra).

Phospholipid and Inositol-Phosphate Signaling

Inositol phospholipids (phosphoinositides) are involved in an intracellular signaling pathway that begins with binding of a signaling molecule to a G-protein linked receptor in the plasma membrane. This leads to the phosphorylation of phosphatidylinositol (PI) residues on the inner side of the plasma membrane to the biphosphate state (PIP₂) by inositol kinases. Simultaneously, the G-protein linked receptor binding stimulates a trimeric G-protein which in turn activates a phosphoinositide-specific phospholipase C-β. Phospholipase C-β then cleaves PIP₂ into two products, inositol triphosphate (IP₃) and diacylglycerol. These two products act as mediators for separate signaling events. IP₃ diffuses through the plasma membrane to induce calcium release from the endoplasmnic reticulum (ER), while diacylglycerol remains in the membrane and helps activate protein kinase C, a serine-threonine linase that phosphorylates selected proteins in the target cell. The calcium response initiated by IP₃ is terminated by the dephosphorylation of IP₃ by specific inositol phosphatases. Cellular responses that are mediated by this pathway are glycogen breakdown in the liver in response to vasopressin, smooth muscle contraction in response to acetylcholine, and thrombin-induced platelet aggregation.

Inositol-phosphate signaling controls tubby, a membrane bound transcriptional regulator that serves as an intracellular messenger of Ge-coupled receptors (Santagata et al. (2001) Science 292:2041-2050). Members of the tubby family contain a C-terminal tubby domain of about 260 amino acids that binds to double-stranded DNA and an N-terminal transcriptional activation domain. Tabby binds to phosphatidylinositol 4,5-bisphosphate, which localizes tubby to the plasma membrane. Activation of the G-protein t leads to activation of phospholipase C-β and hydrolysis of phosphoinositide. Loss of phosphatidylinositol 4,5-bisphosphate causes tubby to dissociate from the plasma membrane and to translocate to the nucleus where tubby regulates transcription of its target genes. Defects in the tubby gene are associated with obesity, retinal degeneration, and hearing loss (Boggon, T. J. et al. (1999) Science 286:2119-2125).

Cyclic Nucleotide Signaling

Cyclic nucleotides (cAMP and cGMP) function as intracellular second messengers to transduce a variety of extracellular signals including hormones, light, and neurotransmitters. In particular, cyclic-AMP dependent protein kinases (PKA) are thought to account for all of the effects of cAMP in most mammalian cells, including various hormone-induced cellular responses. Visual excitation and the phototransmission of light signals in the eye is controlled by cyclic-GMP regulated, Ca²+-specific channels. Because of the importance of cellular levels of cyclic nucleotides in mediating these various responses, regulating the synthesis and breakdown of cyclic nucleotides is an important matter. Thus adenylyl cyclase, which synthesizes cAMP from AMP, is activated to increase cAMP levels in muscle by binding of adrenaline to β-adrenergic receptors, while activation of guanylat cyclase and increased cGMP levels in photoreceptors leads to reopening of the Ca²⁺-specific channels and recovery of the dark state in the eye. There are nine known transmembrane isoforms of mammalian adenylyl cyclase, as well as a soluble form preferentially expressed in testis. Soluble adenylyl cyclase contains a P-loop, or nucleotide binding domain, and may be involved in male fertility (Buck, J. et al. (1999) Proc. Natl. Acad. Sci. USA 96:79-84).

In contrast, hydrolysis of cyclic nucleotides by cAMP and cGMP-specific phosphodiesterases (PDEs) produces the opposite of these and other effects mediated by increased cyclic nucleotide levels. PDEs appear to be particularly important in the regulation of cyclic nucleotides, considering the diversity found in this family of proteins. At least seven families of mammalian PDEs (PDE1-7) have been identified based on substrate specificity and affinity, sensitivity to cofactors, and sensitivity to inhibitory drugs (Beavo, J. A. (1995) Physiol. Rev. 75:725-748). PDE inhibitors have been found to be particularly useful in treating various clinical disorders. Rolipram, a specific inhibitor of PD134, has been used in the treatment of depression, and similar inhibitors are undergoing evaluation as anti-inflammatory agents. Theophylline is a nonspecific PDE inhibitor used in the treatment of bronchial asthma and other respiratory diseases (Banner, K. H. and C. P. Page (1995) Eur. Respir. J. 8:996-1000).

Calcium Signaling Molecules

Ca²⁺ is another second messenger molecule that is even more widely used as an intracellular mediator than cAMP. Ca²⁺ can enter the cytosol by two pathways, in response to extracellular signals. One pathway acts primarily in nerve signal transduction where Ca²⁺ enters a nerve terminal through a voltage-gated Ca²⁺ channel. The second is a more ubiquitous pathway in which Ca²⁺ is released from the ER into the cytosol in response to binding of an extracellular signaling molecule to a receptor. Ca²⁺ directly activates regulatory enzymes, such as protein kinase C, which trigger signal transduction pathways. Ca²⁺ also binds to specific Ca²⁺-binding proteins (CBPs) such as calmodulin (CaM) which then activate multiple target proteins in the cell including enzymes, membrane transport pumps, and ion channels. CaM interactions are involved in a multitude of cellular processes including, but not limited to, gene regulation, DNA synthesis, cell cycle progression, mitosis, cytokinesis, cytoskeletal organization, muscle contraction, signal transduction, ion homeostasis, exocytosis, and metabolic regulation (Celio, M. R. et al. (1996) Guidebook to Calcium-binding Proteins, Oxford University Press, Oxford, UK, pp. 15-20). Some Ca²⁺ binding proteins are characterized by the presence of one or more EF-hand Ca²⁺ binding motifs, which are comprised of 12 amino acids flanked by α-helices (Celio, supra). The regulation of CBPs has implications for the control of a variety of disorders. Calcineurin, a CaM-regulated protein phosphatase, is a target for inhibition by the immunosuppressive agents cyclosporin and FK506. This indicates the importance of calcineurin and CaM in the immune response and immune disorders (Schwaninger M. et al. (1993) J. Biol Chem. 268:23111-23115). The level of CaM is increased several-fold in tumors and tumor-derived cell lines for various types of cancer (Rasmussen, C. D. and A. R. Means (1989) Trends Neurosci. 12:433-438).

The annexins are a family of calcium-binding proteins that associate with the cell membrane (Towle, C. A. and B. V. Treadwell (1992) J. Biol. Chem. 267:5416-5423). Annexins reversibly bind to negatively charged phospholipids (phosphatidylcholine and phosphatidylserine) in a calcium dependent manner. Annexins participate in various processes pertaining to signal transduction at the plasma membrane, including membrane-cytoskeleton interactions, phospholipase inhibition, anticoagulation, and membrane fusion. Annexins contain four to eight repeated segments of about 60 residues. Each repeat folds-into five alpha helices wound into a right-handed superhelix.

G-Protein Signaling

Guanine nucleotide binding proteins (G-proteins) are critical mediators of signal transduction between a particular class of extracellular receptors, the G-protein coupled receptors (GPCRs), and intracellular second messengers such as cAMP and Ca²⁺. G-proteins are linked to the cytosolic side of a GPCR such that activation of the GPCR by ligand binding stimulates binding of the G-protein to GTP, inducing an “active” state in the G-protein. In the active state, the G-protein acts as a signal to trigger other events in the cell such as the increase of cAMP levels or the release of Ca²⁺ into the cytosol from the ER, which, in turn, regulate phosphorylation and activation of other intracellular proteins. Recycling of the G-protein to the inactive state involves hydrolysis of the bound GTP to GDP by a GTPase activity in the G-protein. (See Alberts, B. et al. (1994) Molecular Biology of the Cell Garland Publishing, Inc. New York, N.Y., pp.734-759.) The superfamily of G-proteins consists of several families which may be grouped as translational factors, heterotrimeric G-proteins involved in transmembrane signaling processes, and low molecular weight (LMW) G-proteins including the proto-oncogene Ras proteins and products of rab, rap, rho, rac, smg21, smg25, YPT, SBC4, and ARF genes, and tubulins (Kaziro, Y. et al (1991) Annu. Rev. Biochem. 60:349400). In all cases, the GTPase activity is regulated through interactions with other proteins.

Heterotrimeric G-proteins are composed of 3 subunits, α, β, and γ, which in their inactive conformation associate as a trimer at the inner face of the plasma membrane. Gabinds GDP or GTP and contains the GTPase activity. The βγ complex enhances binding of Gα to a receptor. Gγ is necessary for the folding and activity of Gβ (Neer, E. J. et al. (1994) Nature 371:297-300). Multiple homologs of each subunit have been identified in mammalian tissues, and different combinations of subunits have specific functions and tissue specificities (Spiegel, A. M. (1997) J. Inher. Metab. Dis. 20:113-121).

The alpha subunits of heterotrimeric G-proteins can be divided into four distinct classes. The α-s class is sensitive to ADP-ribosylation by pertussis toxin which uncouples the receptor:G-protein interaction. This uncoupling blocks signal transduction to receptors that decrease cAMP levels which normally regulate ion channels and activate phospholipases. The inhibitory α-I class is also susceptible to modification by pertussis toxin which prevents α-I from lowering cAMP levels. Two novel classes of α subunits refractory to pertussis toxin modification are a-q, which activates phospholipase C, and α-12, which has sequence homology with the Drosophila gene concertina and may contribute to the regulation of embryonic development (Simon, M. L. (1991) Science 252:802-808).

The mammalian Gβ and Gγ subunits, each about 340 amino acids long, share more than 80% homology. The Gβ subunit (also called transducin) contains seven repeating units, each about 43 amino acids long. The activity of both subunits may be regulated by other proteins such as calmodulin and phosducin or the neural protein GAP 43 (Clapham, D. and E. Neer (1993) Nature 365:403-406). The, β and γ subunits are tightly associated. The β subunit sequences are highly conserved between species, implying that they perform a fundamentally important role in the organization and function of G-protein linked systems (Van der Voorn, L. (1992) FEBS Lett. 307:131-134). They contain seven tandem repeats of the WD-repeat sequence motif, a motif found in many proteins with regulatory functions. WD-repeat proteins contain from four to eight copies of a loosely conserved repeat of approximately 40 amino acids which participates in protein-protein interactions. Mutations and variant expression of β transducin proteins are linked with various disorders. Mutations in LIS1, a subunit of the human platelet activating factor acetylhydrolase, cause Miller-Dieker lissencephaly. RACK1 binds activated protein kinase C, and RbAp48 binds retinoblastoma protein. CstF is required for polyadenylation of mammalian pre-mRNA in vitro and associates with subunits of cleavage-stimulating factor. Defects in the regulation of β-catenin contribute to the neoplastic transformation of human cells. The WD40 repeats of the human F-box protein bTrCP mediate binding to β-catenin, thus regulating the targeted degradation of β-catenin by ubiquitin ligase (Neer et al., supra; Hart, M. et al. (1999) Curr. Biol. 9:207-210). The γ subunit primary structures are more variable than those of the β subunits. They are often post-translationally modified by isoprenylation and carboxyl-methylation of a cysteine residue four amino acids from the C-terminus; this appears to be necessary for the interaction of the βγ subunit with the membrane and with other G-proteins. The βγ subunit has been shown to modulate the activity of isoforms of adenylyl cyclase, phospholipase C, and some ion channels. It is involved in receptor phosphorylation via specific kinases, and has been implicated in the p21ras-dependent activation of the MAP kinase cascade and the recognition of specific receptors by G-proteins (Clapham and Neer, supra).

G-proteins interact with a variety of effectors including adenylyl cyclase (Clapham and Neer, supra). The signaling pathway mediated by cAMP is mitogenic in hormone-dependent endocrine tissues such as adrenal cortex, thyroid, ovary, pituitary, and testes. Cancers in these tissues have been related to a mutationally activated form of a Gα, known as the gsp (Gs protein) oncogene (Dhanasekaran, N. et al. (1998) Oncogene 17:1383-1394). Another effector is phosducin, a retinal phosphoprotein, which forms a specific complex with retinal Gβ and Gγ (Gβγ) and modulates the ability of Gβγ to interact with retinal Gα (Clapham and Neer, supra).

Irregularities in the G-protein signaling cascade may result in abnormal activation of leukocytes and lymphocytes, leading to the tissue damage and destruction seen in many inflammatory and autoimmune diseases such as rheumatoid arthritis, biliary cirrhosis, hemolytic anemia, lupus erythematosus, and thyroiditis. Abnormal cell proliferation, including cyclic AMP stimulation of brain, thyroid, adrenal, and gonadal tissue proliferation is regulated by G proteins. Mutations in Gα subunits have been found in growth-hormone-secreting pituitary somatotroph tumors, hyperfunctioning thyroid adenomas, and ovarian and adrenal neoplasms (Meij, J. T. A. (1996) Mol. Cell Biochem 157:31-38; Aussel, C. et al. (1988) J. Immunol 140:215-220).

LMW G-proteins are GTPases which regulate cell growth, cell cycle control, protein secretion, and intracellular vesicle interaction. They consist of single polypeptides which, like the alpha subunit of the heterotrimeric G-proteins, are able to bind to and hydrolyze GTP, thus cycling between an inactive and an active state. LMW G-proteins respond to extracellular signals from receptors and activating proteins by transducing mitogenic signals involved in various cell functions. The binding and hydrolysis of GTP regulates the response of LMW G-proteins and acts as an energy source during this process (Bokoch, G. M. and C. J. Der (1993) FASEB J. 7:750-759).

At least sixty members of the LMW G-protein superfamily have been identified and are currently grouped into the ras, rho, arf, sar1, ran, and rab subfamilies. Activated ras genes were initially found in human cancers, and subsequent studies confirmed that ras function is critical in determining whether cells continue to grow or become differentiated. Ras1 and Ras2 proteins stimulate adenylate cyclase (Kaziro et al., supra), affecting abroad array of cellular processes. Stimulation of cell surface receptors activates Ras which, in turn, activates cytoplasmic kinases. These kinases translocate to the nucleus and activate key transcription factors that control gene expression and protein synthesis (Barbacid, M. (1987) Annu. Rev. Biochem. 56:779-827; Treisman, R. (1994) Curr. Opin. Genet. Dev. 4:96-98). Other members of the LMW G-protein superfamily have roles in signal transduction that vary with the function of the activated genes and the locations of the G-proteins that initiate the activity. Rho G-proteins control signal transduction pathways that link growth factor receptors to actin polymerization, which is necessary for normal cellular growth and division. The rab, arf, and sar1 families of proteins control the translocation of vesicles to and from membranes for protein processing, localization, and secretion. Vesicle- and target-specific identifiers (v-SNAREs and t-SNAREs) bind to each other and dock the vesicle to the acceptor membrane. The budding process is regulated by the closely related ADP ribosylation factors (ARFs) and SAR proteins, while rab proteins allow assembly of SNARE complexes and may play a role in removal of defective complexes (Rothman, J. and F. Wieland (1996) Science 272:227-234). Ran G-proteins are located in the nucleus of cells and have a key role in nuclear protein import, the control of DNA synthesis, and cell-cycle progression (Hall, A. (1990) Science 249:635-640; Barbacid, supra; Ktistakis, N. (1998) BioEssays 20:495-504; and Sasaki, T. and Y. Takai (1998) Biochem. Biophys. Res. Commun. 245:641-645).

Rab proteins have a highly variable amino terminus containing membrane-specific signal information and a prenylated carboxy terminus which determines the target membrane to which the Rab proteins anchor. More than 30 Rab proteins have been identified in a variety of species, and each has a characteristic intracellular location and distinct transport function. In particular, Rab1 and Rab2 are important in ER-to-Golgi transport; Rab3 transports secretory vesicles to the extracellular membrane; Rab5 is localized to endosomes and regulates the fusion of early endosomes into late endosomes; Rab6 is specific to the Golgi apparatus and regulates intra-Golgi transport events; Rab7 and Rab9 stimulate the fusion of late endosomes and Golgi vesicles with lysosomes, respectively; and Rab10 mediates vesicle fusion from the medial Golgi to the trans Golgi. Mutant forms of Rab proteins are able to block protein transport along a given pathway or alter the sizes of entire organelles. Therefore, Rabs play key regulatory roles in membrane trafficking (Schimmöller, I. S. and S. R. Pfeffer (1998) J. Biol Chem. 243:22161-22164).

The function of Rab proteins in vesicular transport requires the cooperation of many other proteins. Specifically, the membrane-targeting process is assisted by a series of escort proteins (Khosravi-Far, R. et al. (1991) Proc. Natl. Acad. Sci. USA 88:6264-6268). In the medial Golgi, it has been shown that GTP-bound Rab proteins initiate the binding of VAMP-like proteins of the transport vesicle to syntaxin-like proteins on the acceptor membrane, which subsequently triggers a cascade of protein-binding and membrane-fusion events. After transport, GTPase-activating proteins (GAPs) in the target membrane are responsible for converting the GTP-bound Rab proteins to their GDP-bound state. And finally, guanine-nucleotide dissociation inhibitor (GDI) recruits the GDP-bound proteins to their membrane of origin.

The cycling of LMW G-proteins between the GTP-bound active form and the GDP-bound inactive form is regulated by a variety of proteins. Guanosine nucleotide exchange factors (GEFs) increase the rate of nucleotide dissociation by several orders of magnitude, thus facilitating release of GDP and loading with GTP. The best characterized is the mammalian homolog of the Drosophila Son-of-Sevenless protein. Certain Ras-family proteins are also regulated by guanine nucleotide dissociation inhibitors (GDIs), which inhibit GDP dissociation. The intrinsic rate of GTP hydrolysis of the LMW G-proteins is typically very slow, but it can be stimulated by several orders of magnitude by GAPs (Geyer, M. and A. Wittinghofer (1997) Curr. Opin. Struct. Biol. 7:786-792). Both GEF and GAP activity may be controlled in response to extracellular stimuli and modulated by accessory proteins such as RalBP1 and POB1. Mutant Ras-family proteins, which bind but cannot hydrolyze GTPP; are permanently activated, and cause cell proliferation or cancer, as do GEPs that inappropriately activate LMW G-proteins, such as the human oncogene NET1, a Rho-GEF (Drivas, G. T. et al (1990) Mol. Cell Biol. 10:1793-1798; Alberts, A. S. and R. Treisman (1998) EMBO J. 14:4075-4085).

A member of the ARF family of G-proteins is centaurin beta 1A, a regulator of membrane traffic and the actin cytoskeleton. The centaurin β family of GTPase-activating proteins (GAPs) and Arf guanine nucleotide exchange factors contain pleckstrin homology (PE) domains which are activated by phosphoinositides. PH domains bind phosphoinositides, implicating PH domains in signaling processes. Phosphoinositides have a role in converting Arf-GTP to Arf-GDP via the centaurin β family and a role in Arf activation (Kam, J. L. et al (2000) J. Biol. Chem. 275:9653-9663). The rho GAP family is also implicated in the regulation of actin polymerization at the plasma membrane and in several cellular processes. The gene ARHGAP6 encodes GTPase-activating protein 6 isoform 4. Mutations in ARHGAP6, seen as a deletion of a 500 kb critical region in Xp22.3, causes the syndrome microphthalmia with linear skin defects (MIS). MS is an X-linked dominant, male-lethal syndrome (Prakash, S. K. et al. (2000) Hum. Mol. Genet. 9:477-488).

A member of the Rho family of G-proteins is CDC42, a regulator of cytoskeletal rearrangements required for cell division. CDC42 is inactivated by a specific GAP (CDC42GAP) that strongly stimulates the GTPase activity of CDC42 while having a much lesser effect on other Rho family members. CDC42GAP also contains an SH3-binding domain that interacts with the SH3 domains of cell signaling proteins such as p85 alpha and c-Src, suggesting that CDC42GAP may serve as a link between CDC42 and other cell signaling pathways (Barfod, E. T. et al. (1993) J. Biol. Chem. 268:26059-26062).

The Dbl proteins are a family of GEFs for the Rho and Ras G-proteins (Whitehead, I. P. et al. (1997) Biochim. Biophys. Acta 1332:F1-F23). All Dbl family members contain a Dbl homology (DH) domain of approximately 180 amino acids, as well as a pleckstrin homology (PH) domain located immediately C-terminal to the DH domain Most Dbl proteins have oncogenic activity, as demonstrated by the ability to transform various cell lines, consistent with roles as regulators of Rho-mediated oncogenic signaling pathways. The kalrin proteins are neuron-specific members of the Dbl family, which are located to distinct subcellular regions of cultured neurons (Johnson, R. C. (2000) J. Cell Biol. 275:19324-19333).

Other regulators of G-protein signaling (RGS) also exist that act primarily by negatively regulating the G-protein pathway by an unknown mechanism (Druey, K. M. et al. (1996) Nature 379:742-746). Some 15 members of the RGS family have been identified. RGS family members are related structurally through similarities in an approximately 120 amino acid region termed the RGS domain and functionally by their ability to inhibit the interleukin (cytokine) induction of MAP kinase in cultured mammalian 293T cells (Druey et al., supra).

The Immuno-associated nucleotide (LAN) family of proteins has GTP-binding activity as indicated by the conserved ATP/GTP-binding site P-loop motif. The IAN family includes IAN-1, IAN-4, IAP38, and IAG-1. IAN-1 is expressed in the immune system, specifically in T cells and thymocytes. Its expression is induced during thymic events (Poirier, G. M. C. et al. (1999) J. Immunol. 163:4960-4969). IAP38 is expressed in B cells and macrophages and its expression is induced in splenocytes by pathogens. IAG-1, which is a plant molecule, is induced upon bacterial infection (Krucken, J. et al. (1997) Biochem. Biophys. Res. Commun. 230:167-170). IAN-4 is a mitochondrial membrane protein which is preferentially expressed in hematopoietic precursor 32D cells transfected with wild-type versus mutant forms of the bcr/abl oncogene. The bcr/abl oncogene is known to be associated with chronic myelogenous leukemia, a clonal myelo-proliferative disorder, which is due to the translocation between the bcr gene on chromosome 22 and the abl gene on chromosome 9. Bcr is the breakpoint cluster region gene and abl is the cellular homolog of the transforming gene of the Abelson murine leukemia virus. Therefore, the IAN family of proteins appears to play a role in cell survival in immune responses and cellular transformation (Daheron, L. et al. (2001) Nucleic Acids Res. 29:1308-1316).

Formin-related genes (FRL) comprise a large family of morphoregulatory genes and have been shown to play important roles in morphogenesis, embryogenesis, cell polarity, cell migration, and cytokinesis through their interaction with Rho family small GTPases. Formin was first identified in mouse limb deformitity (ld) mutants where the distal bones and digits of all limbs are fused and reduced in size. FRL contains formin homology domains FH1, FH2, and FH3. The FH1 domain has been shown to bind the Src homology 3 (SH3) domain, WWP/WW domains, and profilin. The FH2 domain is conserved and was shown to be essential for formin function as disruption at the FH2 domain results in the characteristic ld phenotype. The FH3 domain is located at the N-terminus of FRL, and is required for associating with Rac, a Rho family GTPase (Yayoshi-Yamamoto, S. et al. (2000) Mol. Cell. Biol 20:6872-6881).

Signaling Complex Protein Domains

PDZ domains were named for three proteins in which this domain was initially discovered. These proteins include PSD-95 (postsynaptic density 95), Dlg (Drosophila lethal(1)discs large-1), and ZO-1 (zonula occludens-1). These proteins play important roles in neuronal synaptic transmission, tumor suppression, and cell junction formation, respectively. Since the discovery of these proteins, over sixty additional PDZ-containing proteins have been identified in diverse prokaryotic and eukaryotic organisms. This domain has been implicated in receptor and ion channel clustering and in the targeting of multiprotein signaling complexes to specialized functional regions of the cytosolic face of the plasma membrane. (For a review of PDZ domain-containing proteins, see Ponting, C. P. et al. (1997) Bioessays 19:469-479.) A large proportion of PDZ domains are found in the eukaryotic MAGUK (membrane-associated guanylate kinase) protein family, members of which bind to the intracellular domains of receptors and channels. However, PDZ domains are also found in diverse membrane-localized proteins such as protein tyrosine phosphatases, serine/threonine kinases, G-protein cofactors, and synapse-associated proteins such as syntrophins and neuronal nitric oxide synthase (nNOS). Generally, about one to three PDZ domains are found in a given protein, although up to nine PDZ domains have been identified in a single protein. The glutamate receptor interacting protein (GRIP) contains seven PDZ domains. GRIP is an adaptor that links certain glutamate receptors to other proteins and may be responsible for the clustering of these receptors at excitatory synapses in the brain (Dong, H. et al. (1997) Nature 386:279-284). The Drosophila scribble (SCRIB) protein contains both multiple PDZ domains and leucine-rich repeats. SCRIB is located at the epithelial septate junction, which is analogous to the vertebrate tight junction, at the boundary of the apical and basolateral cell surface. SCRIB is involved in the distribution of apical proteins and correct placement of adherens junctions to the basolateral cell surface (Bilder, D. and N. Perrimon (2000) Nature 403:676-680).

The PX domain is an example of a domain specialized for promoting protein-protein interactions. The PX domain is found in sorting nexins and in a variety of other proteins, including the PhoX components of NADPH oxidase and the Cpk class of phosphatidylinositol 3-kinase. Most PX domains contain a polyproline motif which is characteristic of SH3 domain-binding proteins (Ponting, C. P. (1996) Protein Sci. 5:2353-2357). SH3 domain-mediated interactions involving the PhoX components of NADPH oxidase play a role in the formation of the NADPH oxidase multi-protein complex (Leto, T. L. et al. (1994) Proc. Natl. Acad. Sci. USA 91:10650-10654; Wilson, L. et al. (1997) Inflamm. Res. 46:265-271).

The SH3 domain is defined by homology to a region of the proto-oncogene c-Src, a cytoplasmic protein tyrosine kinase. SH3 is a small domain of 50 to 60 amino acids that interacts with proline-rich ligands. SH3 domains are found in a variety of eukaryotic proteins involved in signal transduction, cell polarization, and membrane-cytoskeleton interactions. In some cases, SH3 domain-containing proteins interact directly with receptor tyrosine kinases. For example, the SLAP-130 protein is a substrate of the T-cell receptor (TCR) stimulated protein kinase. SLAP-130 interacts via its SH-3 domain with the protein SLP-76 to affect the TCR-induced expression of interleukin-2 (Musci, M. A. et al. (1997) J. Biol. Chem. 272:11674-11677). Another recently identified S53 domain protein is macrophage actin-associated tyrosine-phosphorylated protein (MAYP) which is phosphorylated during the response of macrophages to colony stimulating factor-1 (CSF-1) and is likely to play a role in regulating the CSF-1-induced reorganization of the actin cytoskeleton (Yeung, Y.-G. et al (1998) J. Biol. Chem. 273:30638-30642). The structure of the SH3 domain is characterized by two antiparallel beta sheets packed against each other at right angles. This packing forms a hydrophobic pocket lined with residues that are highly conserved between different SH3 domains. This pocket makes critical hydrophobic contacts with proline residues in the ligand (Feng, S. et al. (1994) Science 266:1241-1247).

A novel domain, called the WW domain, resembles the SH3 domain in its ability to bind proline-rich ligands. This domain was originally discovered in dystrophin, a cytoskeletal protein with direct involvement in Duchenne muscular dystrophy (Bork, P. and M. Sudol (1994) Trends Biochem. Sci. 19:531-533). WW domains have since been discovered in a variety of intracellular signaling molecules involved in development, cell differentiation, and cell proliferation. The structure of the WW domain is composed of beta strands grouped around four conserved aromatic residues, generally tryptophan.

Like SH3, the SH2 domain is defined by homology to a region of c-Src. SH2 domains interact directly with phospho-tyrosine residues, thus providing an immediate mechanism for the regulation and transduction of receptor tyrosine kinase-mediated signaling pathways. For example, as many as ten distinct SH2 domains are capable of binding to phosphorylated tyrosine residues in the activated PDGF receptor, thereby providing a highly coordinated and finely tuned response to ligand-mediated receptor activation. (Reviewed in Schaffhausen, B. (1995) Biochim. Biophys. Acta. 1242:61-75.) The BLNK protein is a linker protein involved in B cell activation, that bridges B cell receptor-associated kinases with SH2 domain effectors that link to various signaling pathways (Fu, C. et al. (1998) Immunity 9:93-103).

The pleckstrin homology (PH) domain was originally identified in pleckstrin, the predominant substrate for protein kinase C in platelets. Since its discovery, this domain has been identified in over 90 proteins involved in intracellular signaling or cytoskeletal organization. Proteins containing the pleckstrin homology domain include a variety of kinases, phospholipase-C isoforms, guanine nucleotide release factors, and GTPase activating proteins. For example, members of the FGD1 family contain both Rho-guanine nucleotide exchange factor (GEF) and PH domains, as well as a FYVE zinc finger domain. FGD1 is the gene responsible for faciogenital dysplasia, an inherited skeletal dysplasia (Pasteris, N. G. and J. L. Gorski (1999) Genomics 60:57-66). Many PH domain proteins function in association with the plasma membrane, and this association appears to be mediated by the PH domain itself. PH domains share a common structure composed of two antiparallel beta sheets flanked by an amphipathic alpha helix Variable loops connecting the component beta strands generally occur within a positively charged environment and may function as ligand binding sites (Lemmon, M. A. et al. (1996) Cell 85:621-624). Ankyrin (ANK) repeats mediate protein-protein interactions associated with diverse intracellular signaling functions. For example, ANK repeats are found in proteins involved in cell proliferation such as kinases, kinase inhibitors, tumor suppressors, and cell cycle control proteins. (See, for example, Kalus, W. et al. (1997) FEBS Lett. 401:127-132; Ferrante, A. W. et al (1995) Proc. Natl. Acad. Sci. USA 92:1911-1915.) These proteins generally contain multiple ANK repeats, each composed of about 33 amino acids. Myotrophin is an ANK repeat protein that plays a key role in the development of cardiac hypertrophy, a contributing factor to many heart diseases. Structural studies show that the myotrophin ANK repeats, like other ANK repeats, each form a helix-turn-helix core preceded by a protruding “tip.” These tips are of variable sequence and may play a role in protein-protein interactions. The helix-turn-helix region of the ANK repeats stack on top of one another and are stabilized by hydrophobic interactions (Yang, Y. et al. (1998) Structure 6:619-626). Members of the ASB protein family contain a suppressor of cytokine signaling (SOCS) domain as well as multiple ankyrin repeats (Hilton, D. J. et al. (1998) Proc. Natl. Acad. Sci. USA 95:114-119).

The tetratricopeptide repeat (TPR) is a 34 amino acid repeated motif found in organisms from bacteria to humans. TPRs are predicted to form ampipathic helices, and appear to mediate protein-protein interactions. TPR domains are found in CDC16, CDC23, and CDC27, members of the anaphase promoting complex which targets proteins for degradation at the onset of anaphase. Other processes involving TPR proteins include cell cycle control, transcription repression, stress response, and protein kinase inhibition (Lamb, J. R. et al (1995) Trends Biochem. Sci. 20:257-259).

The armadillo/beta-catenin repeat is a 42 amino acid motif which forms a superhelix of alpha helices when tandemly repeated. The structure of the armadillo repeat region from beta-catenin revealed a shallow groove of positive charge on one face of the superhelix, which is a potential binding surface. The armadillo repeats of beta-catenin, plakoglobin, and p120^{cas} bind the cytoplasmnic domains of cadherins. Beta-cateninicadherin complexes are targets of regulatory signals that govern cell adhesion and mobility (Huber, A. R et al (1997) Cell 90:871-882).

Eight tandem repeats of about 40 residues (WD-40 repeats), each containing a central Trp-Asp motif, make up beta-transducin (G-beta), which is one of the three subunits (alpha, beta, and gamma) of the guanine nucleotide-binding proteins (G proteins). In higher eukaryotes G-beta exists as a small multigene family of highly conserved proteins of about 340 amino acid residues.

COP1 (constitutive photomorphogenic protein) from plants and PML (promyelocytic leukemia protein) from mammals both contain RING-fingers and have similarities in cellular distribution, dynamics, and structure. They possibly function in regulating the targeting of nuclear proteins to specific nuclear compartments for degradation through the ubiquitin-proteasome pathway keyes, J. C. (2001) Trends Biochem. Sci. 26:18-20). More specifically, in the dark, COP1 accumulates in the plant nucleus where it functions in the degradation of the HY5 protein, a positive regulator of photomorphogenesis. In the light, COP1 is excluded from the nucleus allowing the constitutively nuclear HY5 protein to accumulate (Schwechheimer, C. and Deng, X. W. (2000) Semin. Cell Dev. Biol. 11:495-503).

The Gab2 protein is a scaffolding protein attaching to inositol lipids at the cytoplasmic face of the plasma membrane through its PH domain. Gab2 contains a pleckstrin homology domain, and potential binding sites for proteins containing SH2- and SH3-domains as well as for 14-3-3 proteins. Gab2, like DOS (daughter of sevenless) in Drosophila, controls the development of cells. Gab2 acts downstream of a broad range of cytokine, growth factor receptors, and the T and B antigen receptors, linking these receptors to MAP kinase by somehow switching between the MAP kinase pathway and the GAB2 mediated pathway (See http://www.fhcrc.org/labs/rohrschneider/GabPagel.html; Liu, Y. et al. (2001) Mol. Cell Biol. 21:3047-3056; and Crouin, C. et al. (2001) FEBS Let. 495:148-153).

The epidermal growth factor (EGF) superfamily is a diverse group of proteins that function as secreted signaling molecules, growth factors, and components of the extracellular matrix, which are involved in vertebrate development. The Scube1 (signal peptide-CUB domain-EGF-related 1) gene is a novel mammalian gene encoding an EGF-related protein with a CUB (C1s-like) domain that defines a new mammalian gene family. The Scube1 gene is on chromosome 15 and is expressed in developing gonad, nervous system, somites, surface ectoderm, and limb buds. It is similar to a human gene in the syntenic region of chromosome 22q13 (Grimmond, S. (2000) Genomics 70:74-81).

Intracellular Trafficking Proteins

Eukaryotic cells are bound by a lipid bilayer membrane and subdivided into functionally distinct, membrane-bound compartments. The membranes maintain the essential differences between the cytosol, the extracellular environment, and the lumenal space of each intracellular organelle. Eukaryotic proteins including integral membrane proteins, secreted proteins, and proteins destined for the lumen of organelles are synthesized within the endoplasmic reticulum (ER), delivered to the Golgi complex for post-translational processing and sorting, and then transported to specific intracellular and extracellular destinations. Material is internalized from the extracellular environment by endocytosis, a process essential for transmission of neuronal, metabolic, and proliferative signals; uptake of many essential nutrients; and defense against invading organisms. This intracellular and extracellular movement of protein molecules is termed vesicle trafficking. Trafficking is accomplished by the packaging of protein molecules into specialized vesicles which bud from the donor organelle membrane and fuse to the target membrane (Rothman, J. E and Wieland, F. T. (1996) Science 272:227-234).

Several steps in the transit of material along the secretory and endocytic pathways require the formation of transport vesicles. Specifically, vesicles form at the transitional endoplasmic reticulum (tER), the rim of Golgi cisternae, the face of the Trans-Golgi Network (TGN), the plasma membrane (PM), and tubular extensions of the endosomes. Vesicle formation occurs when a region of membrane buds off from the donor organelle. The membrane-bound vesicle contains proteins to be transported and is surrounded by a proteinaceous coat, the components of which are recruited from the cytosol The initial budding and coating processes are controlled by a cytosolic ras-like GTP-binding protein, ADP-ribosylating factor (Arf), and adapter proteins (AP). Cytosolic GTP-bound Arf is also incorporated into the vesicle as it forms. Different isoforms of both Arf and AP are involved at different sites of budding. For example, Arfs 1, 3, and 5 are required for Golgi budding, Arf4 for endosomal budding, and Arf6 for plasma membrane budding. Two different classes of coat protein have also been identified. Clathrin coats form on vesicles derived from the TGN and PM, whereas coatomer (COP) coats form on vesicles derived from the ER and Golgi (Mellman, I. (1996) Annu. Rev. Cell Dev. Biol. 12:575-625).

In clathrin-based vesicle formation, APs bring vesicle cargo and coat proteins together at the surface of the budding membrane. APs are heterotetrameric complexes composed of two large chains: one chain comprised of an α, γ, δ, or ε chain with a β chain, a medium chain (μ), and a small chain (σ). Clathrin binds to APs via the carboxy-terminal appendage domain of the β-adaptin subunit (Le Bourgne, R. and Hoflack, B. (1998) Curr. Opin. Cell. Biol 10:499-503). AP-1 functions in protein sorting from the TGN and endosomes to compartments of the endosomal/lysosomal system. AP-2 functions in clathrin-mediated endocytosis at the plasma membrane, while AP-3 is associated with endosomes and/or the TGN and recruit& integral membrane proteins for transport to lysosomes and lysosome-related organelles. The recently isolated AP-4 complex localizes to the TGN or a neighboring compartment and may play a role in sorting events thought to take place in post-Golgi compartments (Dell'Angelica, E. C. et al. (1999) J. Biol. Chem. 274:7278-7285). Cytosolic GTP-bound Arf is also incorporated into the vesicle as it forms. Another GTP-binding protein, dynamin, forms a ring complex around the neck of the forming vesicle and provides the mechanochemical force required to release the vesicle from the donor membrane. The coated vesicle complex is then transported through the cytosol. During the transport process, Arf-bound GTP is hydrolyzed to GDP and the coat dissociates from the transport vesicle (West, M. A. et al (1997) J. Cell Biol. 138:1239-1254).

Coatomer (COP) coats, a second class of coat proteins, form on vesicles derived from the ER and Golgi. COP coats can further be classified as COPI, involved in retrograde traffic through the Golgi and from the Golgi to the ER, and COPII, involved in anterograde traffic from the ER to the Golgi (Mellman, supra). The COP coat consists of two major components, a GTP-binding protein (Arf or Sar) and coat protomer (coatomer). Coatomer is an equimolar complex of seven proteins, termed α-, β-, βα-, γ-, Δ-, ε- and Z-COP. The coatomer complex binds to dilysine motifs contained on the cytoplasmic tails of integral membrane proteins. These include the dilysine-containing retrieval motif of membrane proteins of the ER and dibasic/diphenylamine motifs of members of the p24 family. The p24 family of type I membrane proteins represents the major membrane proteins of COPI vesicles. (Harter, C. and Wieland, F. T. (1998) Proc. Natl. Acad. Sci. USA 95:11649-11654.)

Vesicles can undergo homotypic, fusing with a same type vesicle, or heterotypic, fusing with a different type vesicle, fusion. Molecules required for appropriate targeting and fusion of vesicles include proteins in the vesicle membrane, the target membrane, and proteins recruited from the cytosol. During budding of the vesicle from the donor compartment, an integral membrane protein, VAMP (vesicle-associated membrane protein) is incorporated into the vesicle. Soon after the vesicle uncoats, a cytosolic prenylated GTP-binding protein, Rab, is inserted into the vesicle membrane. The amino acid sequence of Rab proteins reveals conserved GTP-binding domains characteristic of Ras superfamily members. In the vesicle membrane, GTP-bound Rab interacts with VAMP. Once the vesicle reaches the target membrane, a GTPase activating protein (GAP) in the target membrane converts the Rab protein to the GDP-bound form. A cytosolic protein, guanine-nucleotide dissociation inhibitor (GDI) then removes GDP-bound Rab from the vesicle membrane. Several Rab isoforms have been identified and appear to associate with specific compartments within the cell. For example, Rabs, 4, 5, and 11 are associated with the early endosome, whereas Rabs 7 and 9 associate with the late endosome. These differences may provide selectivity in the association between vesicles and their target membranes. (Novick, P., and Zerial, M. (1997) Cur. Opin. Cell Biol. 9:496-504.)

Docking of the transport vesicle with the target membrane involves the formation of a complex between the vesicle SNAP receptor (v-SNARE), target membrane (t-) SNAREs, and certain other-membrane and cytosolic proteins. Many of these other proteins have been identified although their exact functions in the docling complex remain uncertain (Tellam, J. T. et al. (1995) J. Biol. Chem. 270:5857-5863; Hata, Y. and Sudhof, T. C. (1995) J. Biol. Chem. 270:13022-13028). N-ethylmaleimide sensitive factor (NSF) and soluble NSF-attachment protein (α-SNAP and β-SNAP) are two such proteins that are conserved from yeast to man and function in most intracellular membrane fusion reactions. Sec1 represents a family of yeast proteins that function at many different stages in the secretory pathway including membrane fusion. Recently, mammalian homologs of Sec1, called Munc-18 proteins, have been identified (Katagiri, X et al. (1995) J. Biol Chem. 270:4963-4966; Hata et al. sutra).

The SNARE complex involves three SNARE molecules, one in the vesicular membrane and two in the target membrane. Together they form a rod-shaped complex of four α-helical coiled-coils. The membrane anchoring domains of all three SNAREs project from one end of the rod. This complex is similar to the rod-like structures formed by fusion proteins characteristic of the enveloped viruses, such as myxovirus, influenza, filovirus (Ebola), and the HIV and SIV retroviruses (Skehel, J. J., and Wiley, D. C. (1998) Cell 95:871-874). It has been proposed that the SNARE complex is sufficient for membrane fusion, suggesting that the proteins which associate with the complex provide regulation over the fusion event (Weber, T. et al. (1998) Cell 92:759-772). For example, in neurons, which exhibit regulated exocytosis, docked vesicles do not fuse with the presynaptic membrane until depolarization, which leads to an influx of calcium (Bennett, M. K., and Scheller, R. H. (1994) Annu. Rev. Biochem. 63:63-100). Synaptotagmin, an integral membrane protein in the synaptic vesicle, associates with the t-SNARE syntaxin in the docking complex. Synaptotagmin binds calcium in a complex with negatively charged phospholipids, which allows the cytosolic SNAP protein to displace synaptotagmin from syntaxin and fusion to occur. Thus, synaptotagmin is a negative regulator of fusion in the neuron. (Littleton, J. T. et al. (1993) Cell 74:1125-1134.)

In many cases the tSNARE exists as a complex of syntaxin with a member of the syntaptosome-associated protein-25 (SNAP-25) family of palmitoylated proteins. In neurons and neuroendocrine cells, the tSNAREs consist of syntaxin and SNAP-25, while SNAP-23 replaces SNAP-25 in nonneuronal tissues (Ravichindran, V. et al (1996) J. Biol. Chem. 271:13300-13303). The human SNAP-23 gene was recently mapped to human chromosome region 15q15-21. Several neurological syndromes have been mapped to this region, including some forms of schizophrenia, autism, epilepsy, and a variant of late infantile neuronal ceroid lipofuccinosis in which there is an accumulation of large intracellular vesicles. Alterations of membrane fusion proteins is highly likely to result in distinct clinical syndromes, as in the case of Williams syndrome, a neurological defect resulting from hemizygous deletions of the syntaxin 1A gene. Therefore SNAP-23 is considered to be a candidate gene for any of the neurological syndromes that map to its region of human chromosome 15 (Lazo, P. A. et al. (2001) Hum. Genet. 108:211-215).

The etiology of numerous other human diseases and disorders can be attributed to defects in the trafficking of proteins to organelles or the cell surface. Defects in the trafficking of membrane-bound receptors and ion channels are associated with cystic fibrosis (cystic fibrosis transmembrane conductance regulator; CFTR), glucose-galactose malabsorption syndrome (Na+/glucose cotransporter), hypercholesterolemia (low-density lipoprotein (LDL) receptor), and forms of diabetes mellitus (insulin receptor). Abnormal hormonal secretion is linked to disorders including diabetes insipidus (vasopressin), hyper- and hypoglycemia (insulin, glucagon), Grave's disease and goiter (thyroid hormone), and Cushing's and Addison's diseases (adrenocorticotropic hormone; ACTH).

Cancer cells secrete excessive amounts of hormones or other biologically active peptides. Disorders related to excessive secretion of biologically active peptides by tumor cells include: fasting hypoglycemia due to increased insulin secretion from insulinoma-islet cell tumors; hypertension due to increased epinephrine and norepinephrine secreted from pheochromocytomas of the adrenal medulla and sympathetic paraganglia; and carcinoid syndrome, which includes abdominal cramps, diarrhea, and valvular heart disease, caused by excessive amounts of vasoactive substances (serotonin, bradykinin, histamine, prostaglandins, and polypeptide hormones) secreted from intestinal tumors. Ectopic synthesis and secretion of biologically active peptides (peptides not expected from a tumor) includes s ACTH and vasopressin in lung and pancreatic cancers; parathyroid hormone in lung and bladder cancers; calcitonin in lung and breast cancers; and thyroid-stimulating hormone in medullary thyroid carcinoma.

Various human pathogens alter host cell protein trafficking pathways to their own advantage. For example, the HIV protein Nef down-regulates cell surface expression of CD4 molecules by accelerating their endocytosis through clathrin coated pits. This function of Nef is important for the spread of H[V from the infected cell (Harris, M. (1999) Curr. Biol. 9:R449-R461). A recently identified human protein, Nef-associated factor 1 (Naf1), a protein with four extended coiled-coil domains, has been found to associate with Nef. Overexpression of Naf1 increased cell surface expression of CD4, an effect which could be suppressed by Nef (Fukushi, M. et al. (1999) FEBS Lett. 442:83-88).

PACS-1 (phosphofurin acidic cluster sorting protein-1) controls the endosome to Golgi trafficking of integral membrane proteins that contain acidic cluster sorting motifs, including furin, Nef, and herpes virus envelope glycoproteins, by connecting the acidic cluster domains of these proteins with AP-1. Nef downregulates the surface expression of major histocompatibility complex class I (MHC-1) proteins, thereby promoting immune evasion by HN-1. This process is dependent upon binding of Nef to PACS-1 (Piguet, V. et al. (2000) Nature Cell Biol. 2:163-167). A PACS-1 mutant altered in the adaptor binding site was able to disrupt the Nef-dependent redistribution of MHC-1, suggesting the possibility of controlling IRV immune evasion through inlubtion of PACS-1 (Crump, C. M. et al. (2001) EMBO J. 20:2191-2201).

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

The potential application of gene expression profiling is particularly relevant to improving the diagnosis, prognosis, and treatment of cancers, such as lung cancer.

Lung Cancer

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium. In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas maybe related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exbibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in hug cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium. Among the known genes they identified were keratin isoform 6, KOC, SPRC, IGFb2, connexin 26, plakofillin 1 and cytokeratin 13.

Alzheimer's Disease

The potential application of gene expression profiling is also particularly relevant to improving diagnosis, prognosis, and treatment of neurological disorders, such as Alzheimer's disease (AD). Characterization of region-specific gene expression in the human brain provides a context and background for molecular neurobiology on a variety of neurological disorders. For example, AD is a progressive, neurodestructive process of the human neocortex, characterized by the deterioration of memory and higher cognitive function. A progressive and irreversible brain disorder, AD is characterized by three major pathogenic episodes involving (a) an aberrant processing and deposition of beta-amyloid precursor protein (betaAPP) to form neurotoxic beta-amyloid (betaA) peptides and an aggregated insoluble polymer of betaA that forms the senile plaque, (b) the establishment of intraneuronal neuritic tau pathology yielding widespread deposits of agyrophilic neurofibrillary tangles (NFT) and (c) the initiation and proliferation of a brain-specific inflammatory response. These three seemingly disperse attributes of AD etiopathogenesis are linked by the fact that proinflammatory microglia, reactive astrocytes and their associated cytokines and chemokines are associated with the biology of the microtubule associated protein tan, betaA speciation and aggregation. Missense mutations in the presenilin genes PS1 and PS2, implicated in early onset familial AD, cause abnormal betaAPP processing with resultant overproduction of betaA42 and related neurotoxic peptides. Specific betaA fragments such as betaA42 can further potentiate proinflammatory mechanisms. Expression of the inducible oxidoreductase cyclooxygenase-2 and cytosolic phospholipase A2 (cPLA2) are strongly activated during cerebral ischemia and trauma, epilepsy and AD, indicating the induction of proinflammatory gene pathways as a response to brain injury. Neurotoxic metals such as aluminum and zinc, both implicated in AD etiopathogenesis, and arachidonic acid, a major metabolite of brain cPLA2 activity, each polymerize hyperphosphorylated tan to form NFT-like bundles. Studies have identified a reduced risk for AD in patients aged over 70 years who were previously treated with non-steroidal anti-inflammatory drugs for non-CNS afflictions that include arthritis. (For a review of the interrelationships between the mechanisms of PS1, PS2 and betaAPP gene expression, tau and betaA deposition and the induction, regulation and proliferation in AD of the neuroinflammatory response, see Lukiw W. J, and Bazan N. G.(2000) Neurochem. Res. 2000 25:1173-1184).

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

There is a need in the art for new compositions, including nucleic acids and proteins, for the diagnosis, prevention, and treatment of cell proliferative, autoimmune/inflammatory, neurological, gastrointestinal, reproductive, developmental, and vesicle trafficking disorders.

### SUMMARY OF THE INVENTION

Various embodiments of the invention provide purified polypeptides, intracellular signaling molecules, referred to collectively as “INTSIG” and individually as “INTSIG-1,” “INTSIG-2,” ΘINTSIG-3,” “INTSIG-4,” “INTSIG-5,” “INTSIG-6,” “INTSIG-7,” “INTSIG-8,” “INTSIG-9,” “INTSIG-10,” “INTSIG-11,” “INTSIG-12,” “INTSIG-13,” “INTSIG-14,” “INTSIG-15,” “INTSIG-16,” “INTSIG-17,” “INTSIG-18,” “INTSIG-19,” and ”INTSIG-20,” and methods for using these proteins and their encoding polynucleotides for the detection, diagnosis, and treatment of diseases and medical conditions. Embodiments also provide methods for utilizing the purified intracellular signaling molecules and/or their encoding polynucleotides for facilitating the drug discovery process, including determination of efficacy, dosage, toxicity, and pharmacology. Related embodiments provide methods for utilizing the purified intracellular signaling molecules and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO: 1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides an isolated polypeptide comprising an amino acid sequence of SEQ ID NO:1-20.

Still another embodiment provides an isolated polynucleotide encoding a polypeptide selected from the group consisting of a) a polypeptide comprising an amino acid sequence selected from the group consisting of SEQ ID NO: 1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. In another embodiment, the polynucleotide encodes a polypeptide selected from the group consisting of SEQ ID NO:1-20. In an alternative embodiment, the polynucleotide is selected from the group consisting of SEQ ID NO:21-40.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ D) NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20.

Still yet another embodiment provides an isolated polynucleotide selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:21-40, b) a polynucleotide comprising a naturally occurring polynucleotide sequence at least 90% identical or at least about 90% identical to a polynucleotide sequence selected from the group consisting of SEQ ID NO:21-40, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO: 1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO: 1-20, and a pharmaceutically acceptable excipient In one embodiment, the composition can comprise an amino acid sequence selected from the group consisting of SEQ ID NO: 1-20. Other embodiments provide a method of treating a disease or condition associated with decreased or abnormal expression of functional INTSIG, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) abiologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional INTSIG, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional INTSIG, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-20, b) a polypeptide comprising a naturally occurring amino acid sequence at least 90% identical or at least about 90% identical to an amino acid sequence selected from the group consisting of SEQ ID NO:1-20, c) a biologically active fragment of a polypeptide having an amino acid sequence selected from the group consisting of SEQ ID NO:1-20, and d) an immunogenic fragment of a polypeptide having an amino acid sequence selected from the group consisting of SEQ ID NO:1-20. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:21-40, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:21-40, ii) a polynucleotide comprising a naturally occurring polynucleotide sequence at least 90% identical or at least about 90% identical to a polynucleotide sequence selected from the group consisting of SEQ ID NO:21-40, iii) a polynucleotide having a sequence complementary to i), iv) a polynucleotide complementary to the polynucleotide of in), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:21-40, ii) a polynucleotide comprising a naturally occurring polynucleotide sequence at least 90% identical or at least about 90% identical to a polynucleotide sequence selected from the group consisting of SEQ ID NO:21-40, iin) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

Table 8 shows single nucleotide polymorphisms found in polynucleotide embodiments, along with allele frequencies in different human populations.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“INTSIG” refers to the amino acid sequences of substantially purified INTSIG obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of INTSIG. Agonists may include proteins, nucleic acids, carbohydrates, small molecules, or any other compound or composition which modulates the activity of INTSIG either by directly interacting with INTSIG or by acting on components of the biological pathway in which INTSIG participates.

An “allelic variant” is an alternative form of the gene encoding INTSIG. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding INTSIG include those sequences with deletions, insertions, or substitutions of different nucleotides, resulting in a polypeptide the same as INTSIG or a polypeptide with at least one functional characteristic of INTSIG. Included within this definition are polymorphisms which may or may not be readily detectable using a particular oligonucleotide probe of the polynucleotide encoding INTSIG, and improper or unexpected hybridization to allelic variants, with a locus other than the normal chromosomal locus for the polynucleotide encoding INTSIG. The encoded protein may also be “altered,” and may contain deletions, insertions, or substitutions of amino acid residues which produce a silent change and result in a functionally equivalent INTSIG. Deliberate amino acid substitutions may be made on the basis of one or more similarities in polarity, charge, solubility, hydrophobicity, hydrophilicity, and/or the amphipathic nature of the residues, as long as the biological or immunological activity of INTSIG is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of INTSIG. Antagonists may include proteins such as antibodies, anticalins, nucleic acids, carbohydrates, small molecules, or any other compound or composition which modulates the activity of INTSIG either by directly interacting with INTSIG or by acting on components of the biological pathway in which INTSIG participates.

The term “antibody” refers to intact immunoglobulin molecules as well as to fragments thereof, such as Fab, F(ab′)₂, and Fv fragments, which are capable of binding an epitopic determinant. Antibodies that bind INTSIG polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole litmpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide maybe replaced by 2′-F or 2′-NH₂, which may improve a desired property, e.g., resistance to nucleases or longer lifetime in blood. Aptamers may be conjugated to other molecules, e.g., a high molecular weight carrier to slow clearance of the aptamer from the circulatory system. Aptamers maybe specifically cross-linked to their cognate ligands, e.g., by photo-activation of a cross-linker. (See, e.g., Brody, E. N. and L. Gold (2000) J. Biotechnol. 74:5-13.)

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic INTSIG, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding INTSIG or fragments of INTSIG may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassorttnent of stable substructures, thus allowing acceleration of the evolution of new protein functions.

A “fragment” is a unique portion of INTSIG or a polynucleotide encoding INTSIG which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or ammo acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:21-40 can comprise a region of unique polynucleotide sequence that specifically identifies SEQ ID NO:21-40, for example, as distinct from any other sequence in the genome from which the fragment was obtained. A fragment of SEQ ID NO:21-40 can be employed in one or more embodiments of methods of the invention, for example, in hybridization and amplification technologies and in analogous methods that distinguish SEQ ID NO:21-40 from related polynucleotides. The precise length of a fragment of SEQ ID NO:21-40 and the region of SEQ ID NO:21-40 to which the fragment corresponds are routinely determinable by one of ordinary skill in the art based on the intended purpose for the fragment.

A fragment of SEQ ID NO:1-20 is encoded by a fragment of SEQ ID NO:21-40. A fragment of SEQ ID NO:1-20 can comprise a region of unique amino acid sequence that specifically identifies SEQ ID NO:1-20. For example, a fragment of SEQ ID NO:1-20 can be used as an immunogenic peptide for the development of antibodies that specifically recognize SEQ ID NO:1-20. The precise length of a fragment of SEQ ID NO:1-20 and the region of SEQ ID NO:1-20 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989) CABIOS 5:151-153 and in Higgins, D. G. et al. (1992) CABIOS 8:189-191. For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nlh.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nlh.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastm with the “BLAST 2 Sequences” tool Version 2.0.12 (April-21-2000) set at default parameters. Such default parameters may be, for example:

Matrix: BLOSUM62

Reward for match: 1

Penalty for mismatch: −2

Open Gap: S and Extension Gap: 2 penalties

Gap x drop-off: 50

Expect: 10

Word Size: 11

Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences maybe determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:

Matrix: BLOSUM62

Open Gap: 11 and Extension Gap: 1 penalties

Gap x drop-off. 50

Expect: 10

Word Size: 3

Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and maybe consistent among hybridization experiments, whereas wash conditions maybe varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2× SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on-a solid support (e.g.; paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively. “Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of INTSIG which is capable of eliciting an immune response when introduced into a living organism, for example, a mammal The term “immunogenic fragment” also includes any polypeptide or oligopeptide fragment of INTSIG which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of INTSIG. For example, modulation may cause an increase or a decrease in protein activity, binding characteristics, or any other biological, functional, or immunological properties of INTSIG.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition PNAs preferentially bind complementary single stranded DNA or RNA and stop transcript elongation, and may be pegylated to extend their lifespan in the cell.

“Post-translational modification” of an INTSIG may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of INTSIG.

“Probe” refers to nucleic acids encoding INTSIG, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, maybe used.

Methods for preparing and using probes and primers are described in the references, for example Sambrook, J. et al. (1989) Molecular Cloning: A Laboratory Manual, 2^{nd} ed., vol. 1-3, Cold Spring Harbor Press, Plainview N.Y.; Ausubel, F. M. et al. (1987) Current Protocols in Molecular Biology, Greene Publ. Assoc. & Wiley-Intersciences, New York N.Y.; Innis, M. et al. (1990) PCR Protocols. A Guide to Methods and Applications, Academic Press, San Diego Calif. PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids maybe part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing INTSIG, nucleic acids encoding INTSIG, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length of one of the polypeptides.

The Invention

Various embodiments of the invention include new human intracellular signaling molecules (INTSIG), the polynucleotides encoding INTSIG, and the use of these compositions for the diagnosis, treatment, or prevention of cell proliferative, autoimmunefmflammatory, neurological, gastrointestinal, reproductive, developmental, and vesicle trafficking disorders.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown.

Table 2 shows sequences with homology to the polypeptides of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are intracellular signaling molecules. For example, SEQ ID NO:1 is 58% identical, from residue M1 to residue R125, to chicken GTPase cRac1B (GenBank ID g3184512) as determined by the Basic Local Alignment Search Tool (BLAST). (See Table 2.) The BLAST probability score is 2.3e-30, which indicates the probability of obtaining the observed polypeptide sequence alignment by chance. Data from BLIMPS, MOTIFS, and additional BLAST analyses provide corroborative evidence that SEQ ID NO:1 is a GTPase. In an alternative example, SEQ ID NO:3 is 37% identical, from residue I63 to residue Y571, to ahuman sorting nexin 9 (GenBankID g4689258) as determined by BLAST. The BLAST probability score is 2.4e-83. In an alternative example, SEQ ID NO:4 is 97% identical, from residue M1 to residue A972, to a murine Rab6-interacting protein (GenBank ID g13445784) as determined by BLAST. The BLAST probability score is 0.0. Data from HMMER-PFAM, BLIMPS, BLAST and MOTIFS analyses provide further corroborative evidence that SEQ ID NO:3 and SEQ ID NO:4 are intracellular signaling molecules. In an alternative example, SEQ ID NO:8 is 98% identical, from residue S75 to residue V731, to mouse COP1 protein (GenBank ID g5762305) as determined by the BLAST. The BLAST probability score is 0.0. SEQ ID NO:8 also contains a WD domain, G-beta repeat, and a zinc finger, C3HC4 type (RING finger) domain as determined by searching for statistically significant matches in the hidden Markov model (MM)-based PFAM database of conserved protein family domains. (See Table 3.) Data from BLIMPS, MOTIFS, and other BLAST analyses against the PRODOM and DOMO databases, provide further corroborative evidence that SEQ ID NO:8 is a COP1 protein, related to promyelocytic leukemia protein (PML) from mammals, both of which are thought to be involved in regulating the targeting of nuclear proteins to specific nuclear compartments for degradation through the ubiquitin-proteasome pathway. In an alternative example, SEQ ID NO:11 is 97% identical, from residue M1 to residue T963, to rat cytosolic sorting protein PACS-1a (GenBank ID g3347953) as determined by the BLAST. The BLAST probability score is 0.0. Data from additional BLAST analyses against the PRODOM database provide further corroborative evidence that SEQ ID NO:11 is a PACS-1 sorting protein. In an alternative example, SEQ ID NO:12 is 100% identical, from residue M1 to residue S158, to human SNAP23B (GenBank ID g1924944) with a BLAST probability score is 2.5e-79. SEQ ID NO:12 also contains a SNAP-25 family domain as determined by searching for statistically significant matches in the HMM-based PFAM database. Data from additional BLAST analyses against the PRODOM database provide further corroborative evidence that SEQ ID NO: 12 is a member of the SNAP-25 family. In an alternative example, SEQ ID NO:13 is 35% identical, from residue D96 to residue F277, to human rhoGAP protein (GenBank ID g312212) as determined by BLAST. The BLAST probability score is 1.3e-20. SEQ ID NO:13 also contains a RhoGAP domain as determined by searching for statistically significant matches in the HMM-based PFAM database. Data from BLIMPS, and additional BLAST analyses provide further corroborative evidence that SEQ ID NO:13 is a GTPase-activating protein (GAP). In an alternative example, SEQ ID NO:15 is 45% identical, from residue E5 to residue B153, to human calmodulin (GenBank ID g179810) as determined by BLAST. The BLAST probability score is 1.3e-29. SEQ ID NO:15 also contains an EF-hand calcium-binding domain as determined by searching for statistically significant matches in the HMM-based PFAM database. Data from BLIMPS and MOTIFS analyses provide further corroborative evidence that SEQ ID NO:15 is a calcium-binding intracellular signaling protein. In an alternative example, SEQ ID NO:18 is 95% identical, from residue M1 to residue L2568, and 99% identical from K2530 to Y2937, to mouse neurobeachin (GenBank ID gl1863685) as determined by BLAST. The BLAST probability score is 0.0. SEQ ID NO:18 also contains a Beige/BEACH domain and multiple WD domains as determined by searching for statistically significant matches in the HMM-based PFAM database. Data from BLIMPS, and additional BLAST analyses provide further corroborative evidence that SEQ ID NO:18 is a WD repeat-containing transport protein. SEQ ID NO:2, SEQ ID NO:5-7, SEQ ID NO:9-10, SEQ ID NO:14, SEQ ID NO:16-17, and SEQ ID NO:19-20 were analyzed and annotated in a similar manner. The algorithms and parameters for the analysis of SEQ ID NO:1-20 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful for example, in hybridization or amplification technologies that identify SEQ ID NO:21-40 or that distinguish between SEQ ID NO:21-40 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT“) or the NCBI RefSeq Protein Sequence Records (ie., those sequences including the designation “NP”) Alternatively, the polynucleotide fragments described in column 2 may refer to assemblages of both cDNA and Genscan-predicted exons brought together by an “exon stitching” algorithm. For example, a polynucleotide sequence identified as FL_XXXXXX_N_{1—}N_{2—}YYYYY_N_{3—}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, Cambridge, |
| | UK). |
| GBI | Hand-edited analysis of genomic sequences. |
| FL | Stitched or stretched genomic sequences (see Example V). |
| INCY | Full length transcript and exon prediction from mapping of EST |
| | sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those fall length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

Table 8 shows single nucleotide polymorphisms (SNPs) found in polynucleotide embodiments, along with allele frequencies in different human populations. Columns 1 and 2 show the polynucleotide sequence identification number (SEQ ID NO:) and the corresponding Incyte project identification number (PID) for polynucleotides of the invention. Column 3 shows the Incyte identification number for the EST in which the SNP was detected (EST ID), and column 4 shows the identification number for the SNP (SNP ID). Column 5 shows the position within the EST sequence at which the SNP is located (EST SNP), and column 6 shows the position of the SNP within the full-length polynucleotide sequence (CB1 SNP). Column 7 shows the allele found in the EST sequence. Columns 8 and 9 show the two alleles found at the SNP site. Column 10 shows the amino acid encoded by the codon including the SNP site, based upon the allele found in the EST. Columns 11-14 show the frequency of allele 1 in four different human populations. An entry of n/d (not detected) indicates that the frequency of allele 1 in the population was too low to be detected, while n/a (not available) indicates that the allele frequency was not determined for the population.

The invention also encompasses INTSIG variants. A preferred INTSIG variant is one which has at least about 80%, or alternatively at least about 90%, or even at least about 95% amino acid sequence identity to the INTSIG amino acid sequence, and which contains at least one functional or structural characteristic of INTSIG.

Various embodiments also encompass polynucleotides which encode INTSIG. In a particular embodiment, the invention encompasses a polynucleotide sequence comprising a sequence selected from the group consisting of SEQ ID NO:21-40, which encodes INTSIG. The polynucleotide sequences of SEQ ID NO:21-40, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding INTSIG. In particular, such a variant polynucleotide will have at least about 70%, or alternatively at least about 85%, or even at least about 95% polynucleotide sequence identity to a polynucleotide encoding INTSIG. A particular aspect of the invention encompasses a variant of a polynucleotide comprising a sequence selected from the group consisting of SEQ ID NO:21-40 which has at least about 70%, or alternatively at least about 85%, or even at least about 95% polynucleotide sequence identity to a nucleic acid sequence selected from the group consisting of SEQ ID NO:21-40. Any one of the polynucleotide variants described above can encode a polypeptide which contains at least one functional or structural characteristic of INTSIG.

In addition, or in the alternative, a polynucleotide variant of the invention is a splice variant of a polynucleotide encoding INTSIG. A splice variant may have portions which have significant sequence identity to a polynucleotide encoding INTSIG, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding INTSIG over its entire length; however, portions of the splice variant will have at least about 70%, or alternatively at least about 85%, or alternatively at least about 95%, or alternatively 100% polynucleotide sequence identity to portions of the polynucleotide encoding INTSIG. For example, a polynucleotide comprising a sequence of SEQ ID NO:37 is a splice variant of a polynucleotide comprising a sequence of SEQ ID NO:40. Any one of the splice variants described above can encode a polypeptide which contains at least one functional or structural characteristic of INTSIG.

It will be appreciated by those skilled in the art that as a result of the degeneracy of the genetic code, a multitude of polynucleotide sequences encoding INTSIG, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring INTSIG, and all such variations are to be considered as being specifically disclosed.

Although polynucleotides which encode INTSIG and its variants are generally capable of hybridizing to polynucleotides encoding naturally occurring INTSIG under appropriately selected conditions of stringency, it maybe advantageous to produce polynucleotides encoding INTSIG or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding INTSIG and its derivatives without altering the encoded amino acid sequences include the production of RNA transcripts having more desirable properties, such as a greater half-life, than transcripts produced from the naturally occurring sequence.

The invention also encompasses production of polynucleotides which encode INTSIG and INTSIG derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding INTSIG or any fragment thereof.

Embodiments of the invention can also include polynucleotides that are capable of hybridizing to the claimed polynucleotides, and, in particular, to those having the sequences shown in SEQ ID NO:21-40 and fragments thereof, under various conditions of stringency. (See, e.g., Wahl, G. M. and S. L. Berger (1987) Methods Enzymol. 152:399-407; Kimmel, A. R. (1987) Methods Enzymol. 152:507-511.) Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PMC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

The nucleic acids encoding INTSIG may be extended utilizing a partial nucleotide sequence and employing various PCR-based methods known in the art to detect upstream sequences, such as promoters and regulatory elements. For example, one method which maybe employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode INTSIG may be cloned in recombinant DNA molecules that direct expression of INTSIG, or fragments or functional equivalents thereof, in appropriate host cells. Due to the inherent degeneracy of the genetic code, other polynucleotides which encode substantially the same or a functionally equivalent polypeptides may be produced and used to express INTSIG.

The polynucleotides of the invention can be engineered using methods generally known in the art in order to alter INTSIG-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat Biotechnol. 17:793-797; Christians, R. C. et al. (1999) Nat. Biotechnol. 17:259-264; and Crameri, A. et al. (1996) Nat. Biotechnol. 14:315-319) to alter or improve the biological properties of INTSIG, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding INTSIG may be synthesized, in whole or in part, using one or more chemical methods well known in the art. (See, e.g., Caruthers, M. H. et al. (1980) Nucleic Acids Symp. Ser. 7:215-223; and Horn, T. et al. (1980) Nucleic Acids Symp. Ser. 7:225-232.) Alternatively, INTSIG itself or a fragment thereof maybe synthesized using chemical methods known in the art. For example, peptide synthesis can be performed using various solution-phase or solid-phase techniques. (See, e.g., Creighton, T. (1984) Proteins, Structures and Molecular Properties, WH Freeman, New York N.Y., pp. 55-60; and Roberge, J. Y. et al (1995) Science 269:202-204.) Automated synthesis maybe achieved using the ABI 431A peptide synthesizer (Applied Biosystems). Additionally, the amino acid sequence of INTSIG, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography. (See, e.g., Chiez, R. M. and F. Z. Regnier (1990) Methods Enzymol. 182:392-421.) The composition of the synthetic peptides may be confirmed by amino acid analysis or by sequencing. (See, e.g., Creighton, supra, pp. 28-53.)

In order to express a biologically active INTSIG, the polynucleotides encoding INTSIG or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding INTSIG. Such elements may vary in their strength and specificity. Specific initiation signals may also be used to achieve more efficient translation of polynucleotides encoding INTSIG. Such signals include the ATG initiation codon and adjacent sequences, e.g. the Kozak sequence. In cases where a polynucleotide sequence encoding INTSIG and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20:125-162.) Methods which are well known to those skilled in the art may be used to construct expression vectors containing polynucleotides encoding INTSIG and appropriate transcriptional and translational control elements. These methods include in vitro recombinant DNA techniques, synthetic techniques, and in vivo genetic recombination. (See, e.g., Sambrook, J. et al. (1989) Molecular Cloning. A Laboratory Manual, Cold Spring Harbor Press, Plainview N.Y., ch 4, 8, and 16-17; Ausubel, F. M. et al. (1995) Current Protocols in Molecular Biology, John Wiley & Sons, New York NY, ch. 9, 13, and 16.)

A variety of expression vector/host systems may be utilized to contain and express polynucleotides encoding INTSIG. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems. (See, e.g., Sambrook, supra; Ausubel, supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; and Harrington, J. J. et al. (199,7) Nat. Genet. 15:345-355.) Expression vectors derived from retroviruses, adenoviruses, or herpes or vaccinia viruses, or from various bacterial plasmids, may be used for delivery of polynucleotides to the targeted organ, tissue, or cell population. (See, e.g., Di Nicola, M. et al. (1998) Cancer Gen. Ther. 5(6):350-356; Yu, M. et al. (1993) Proc. Natl Acad. Sci. USA 90(13):6340-6344; Buller, R. M. et al. (1985) Nature 317(6040):813-815; McGregor, D. P. et al. (1994) Mol. Immunol. 31(3):219-226; and Verma, I. M. and N. Somia (1997) Nature 389:239-242.) The invention is not limited by the host cell employed.

In bacterial systems, a number of cloning and expression vectors may be selected depending upon the use intended for polynucleotides encoding INTSIG. For example, routine cloning, subcloning, and propagation of polynucleotides encoding INTSIG can be achieved using a multifunctional E. coli vector such as PBLUESCRIPT (Stratagene, La Jolla Calif.) or PSPORT1 plasmid Invitrogen). Ligation of polynucleotides encoding INTSIG into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264:5503-5509.) When large quantities of INTSIG are needed, e.g. for the production of antibodies, vectors which direct high level expression of INTSIG may be used. For example, vectors containing the strong, inducible SP6 or T7 bacteriophage promoter maybe used.

Yeast expression systems may be used for production of INTSIG. A number of vectors containing constitutive or inducible promoters, such as alpha factor, alcohol oxidase, and PGH promoters, maybe used in the yeast Saccharomyces cerevisiae or Pichia pastoris. In addition, such vectors direct either the secretion or intracellular retention of expressed proteins and enable integration of foreign polynucleotide sequences into the host genome for stable propagation. (See, e.g., Ausubel, 1995, supra; Bitter, G. A. et al. (1987) Methods Enzymol. 153:516-544; and Scorer, C. A. et al. (1994) Bio/Technology 12:181-184.)

Plant systems may also be used for expression of INTSIG. Transcription of polynucleotides encoding INTSIG maybe driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters maybe used. (See, e.g., Coruzzi, G. et al. (1984) EMBO J. 3:1671-1680; Broglie, R. et al. (1984) Science 224:838-843; and Winter, J. et al. (1991) Results Probl. Cell Differ. 17:85-105.) These constructs can be introduced into plant cells by direct DNA transformation or pathogen-mediated transfection. (See, e.g., The McGraw Hill Yearbook of Science and Technology (1992) McGraw Hill, New York N.Y., pp. 191-196.)

In mammalian cells, a number of viral-based expression systems may be utilized. In cases where an adenovirus is used as an expression vector, polynucleotides encoding INTSIG maybe ligated into an adenovirus transcription/translation complex consisting of the late promoter and tripartite leader sequence. Insertion in a non-essential E1 or E3 region of the viral genome may be used to obtain infective virus which expresses INTSIG in host cells. (See, e.g., Logan, J. and T. Shenk (1984) Proc. Natl. Acad. Sci. USA 81:3655-3659.) In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycatioric amino polymers, or vesicles) for therapeutic purposes. (See, e.g., Harrington, J. J. et al. (1997) Nat. Genet. 15:345-355.).

For long term production of recombinant proteins in mammalian systems, stable expression of INTSIG in cell lines is preferred. For example, polynucleotides encoding INTSIG can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively. (See, e.g., Wigler, M. et al. (1977) Cell 11:223-232; Lowy, L et al. (1980) Cell 22:817-823.) Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively. (See, e.g., Wigler, M. et al. (1980) Proc. Natl. Acad. Sci. USA 77:3567-3570; Colbere-Garapin, F. et al. (1981) J. Mol. Biol. 150:1-14.) Additional selectable genes have been described, e.g., trpB and hisD, which alter cellular requirements for metabolites. (See, e.g., Hartman, S. C. and R. C. Mulligan (1988) Proc. Natl. Acad. Sci. USA 85:8047-8051.) Visible markers, e.g., anthocyanins, green fluorescent proteins (GFP; Clontech), β glucuronidase and its substrate β-glucuronide, or luciferase and its substrate luciferin may be used. These markers can be used not only to identify transformants, but also to quantify the amount of transient or stable protein expression attributable to a specific vector system. (See, e.g., Rhodes, C. A. (1995) Methods Mol. Biol. 55:121-131.)

Although the presence/absence of marker gene expression suggests that the gene of interest is also present, the presence and expression of the gene may need to be confirmed. For example, if the sequence encoding INTSIG is inserted within a marker gene sequence, transformed cells containing polynucleotides encoding INTSIG can be identified by the absence of marker gene function. Alternatively, a marker gene can be placed in tandem with a sequence encoding INTSIG under the control of a single promoter. Expression of the marker gene in response to induction or selection usually indicates expression of the-tandem gene as well.

In general, host cells that contain the polynucleotide encoding INTSIG and that express INTSIG may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences. Immunological methods for detecting and measuring the expression of INTSIG using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on INTSIG is preferred, but a competitive binding assay may be employed. These and other assays are well known in the art. (See, e.g., Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.)

A wide variety of labels and conjugation techniques are known by those skilled in the art and may be used in various nucleic acid and amino acid assays. Means for producing labeled hybridization or PCR probes for detecting sequences related to polynucleotides encoding INTSIG include oligolabeling, nick translation, end-labeling, or PCR amplification using a labeled nucleotide. Alternatively, polynucleotides encoding INTSIG, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures maybe conducted using a variety of commercially available kits, such as those provided by Amersham Biosciences, Promega (Madison Wis.), and US Biochemical. Suitable reporter molecules or labels which maybe used for ease of detection include radionuclides, enzymes, fluorescent, chemiluminescent, or chromogenic agents, as well as substrates, cofactors, inhibitors, magnetic particles, and the like.

Host cells transformed with polynucleotides encoding INTSIG may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode INTSIG maybe designed to contain signal sequences which direct secretion of INTSIG through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WV38) are available from the American Type Culture Collection (ATCC, Manassas Va.) and may be chosen to ensure the correct modification and processing of the foreign protein.

In another embodiment of the invention, natural, modified, or recombinant polynucleotides encoding INTSIG may be ligated to a heterologous sequence resulting in translation of a fusion protein in any of the aforementioned host systems. For example, a chimeric INTSIG protein containing a heterologous moiety that can be recognized by a commercially available antibody may facilitate the screening of peptide libraries for inhibitors of INTSIG activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmoduin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the INTSIG encoding sequence and the heterologous protein sequence, so that INTSIG may be cleaved away from the heterologous moiety following purification Methods for fusion protein expression and purification are discussed in Ausubel (1995, supra, ch. 10). A variety of commercially available kits may also be used to facilitate expression and purification of fusion proteins.

In another embodiment, synthesis of radiolabeled INTSIG maybe achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

INTSIG, fragments of INTSIG, or variants of INTSIG maybe used to screen for compounds that specifically bind to INTSIG. One or more test compounds may be screened for specific binding to INTSIG. In various embodiments, 1, 2, 3, 4, 5, 10, 20, 50, 100, or 200 test compounds can be screened for specific binding to INTSIG. Examples of test compounds can include antibodies, anticalins, oligonucleotides, proteins (e.g., ligands or receptors), or small molecules.

In related embodiments, variants of INTSIG can be used to screen for binding of test compounds, such as antibodies, to INTSIG, a variant of INTSIG, or a combination of INTSIG and/or one or more variants INTSIG. In an embodiment, a variant of INTSIG can be used to screen for compounds that bind to a variant of INTSIG, but not to INTSIG having the exact sequence of a sequence of SEQ ID NO:1-20. INTSIG variants used to perform such screening can have a range of about 50% to about 99% sequence identity to INTSIG, with various embodiments having 60%, 70%, 75%, 80%, 85%, 90%, and 95% sequence identity.

In an embodiment, a compound identified in a screen for specific binding to INTSIG can be closely related to the natural ligand of INTSIG, e.g., a ligand or fragment thereof, a natural substrate, a structural or functional mimetic, or a naturalbinding partner. (See, e.g., Coligan, J. E. et al. (1991) Current Protocols in Immunology 1(2):Chapter 5.) In another embodiment, the compound thus identified can be a natural ligand of a receptor INTSIG. (See, e.g., Howard, A. D. et al. (2001) Trends Pharmacol. Sci.22:132-140; Wise, A. et al. (2002) Drug Discovery Today 7:235-246.)

In other embodiments, a compound identified in a screen for specific binding to INTSIG can be closely related to the natural receptor to which INTSIG binds, at least a fragment of the receptor, or a fragment of the receptor including all or a portion of the ligand binding site or binding pocket. For example, the compound may be a receptor for INTSIG which is capable of propagating a signal, or a decoy receptor for INTSIG which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Immunex Corp., Seattle Wash.), which is efficacious for treating rheumatoid arthritis in humans. Etanercept is an engineered p75 tumor necrosis factor (TNF) receptor dimer linked to the Fc portion of human IgG, (Taylor, P. C. et al. (2001) Curr. Opin. Immunol. 13:611-616).

In one embodiment, two or more antibodies having similar or, alternatively, different specificities can be screened for specific binding to INTSIG, fragments of INTSIG, or variants of INTSIG. The binding specificity of the antibodies thus screened can thereby be selected to identify particular fragments or variants of INTSIG. In one embodiment, an antibody can be selected such that its binding specificity allows for preferential identification of specific fragments or variants of INTSIG. In another embodiment, an antibody can be selected such that its binding specificity allows for preferential diagnosis of a specific disease or condition having increased, decreased, or otherwise abnormal production of INTSIG.

In an embodiment, anticalins can be screened for specific binding to INTSIG, fragments of: INTSIG, or variants of INTSIG. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit INTSIG involves producing appropriate cells which express INTSIG, either as a secreted protein or on the cell membrane. Preferred cells include cells from mammals, yeast, Drosophila, or E. coli. Cells expressing INTSIG or cell membrane fractions which contain INTSIG are then contacted with a test compound and binding, stimulation, or inhibition of activity of either INTSIG or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with INTSIG, either in solution or affixed to a solid support, and detecting the binding of INTSIG to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands. (See, e.g., Matthews, D. J. and J. A. Wells. (1994) Chem. Biol. 1:25-30.) In another related embodiment, one or more amino acid substitutions can be introduced into a polypeptide compound (such as a ligand) to improve or alter its ability to bind to its natural receptors. (See, e.g., Cunningham, B. C. and J. A. Wells (1991) Proc. Natl Acad. Sci. USA 88:3407-3411; Lowman, H. B. et al. (1991) J. Biol. Chem. 266:10982-10988.)

INTSIG, fragments of INTSIG, or variants of INTSIG may be used to screen for compounds that modulate the activity of INTSIG. Such compounds may include agonists, antagonists, or partial or inverse agonists. In one embodiment, an assay is performed under conditions permissive for INTSIG activity, wherein INTSIG is combined with at least one test compound, and the activity of INTSIG in the presence of a test compound is compared with the activity of INTSIG in the absence of the test compound. A change in the activity of INTSIG in the presence of the test compound is indicative of a compound that modulates the activity of INTSIG. Alternatively, a test compound is combined with an in vitro or cell-free system comprising INTSIG under conditions suitable for INTSIG activity, and the assay is performed. In either of these assays, a test compound which modulates the activity of INTSIG may do so indirectly and need not come in direct contact with the test compound. At least one and up to a plurality of test compounds may be screened.

In another embodiment, polynucleotides encoding INTSIG or their mammalian homologs may be knocked out” in an animal model system using homologous recombination in embryonic stem (ES) cells. Such techniques are well known in the art and are useful for the generation of animal models of human disease. (See, e.g., U.S. Pat. No. 5,175,383 and U.S. Pat. No. 5,767,337.) For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (teo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated maybe tested with potential therapeutic or toxic agents.

Polynucleotides encoding INTSIG may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al (1998) Science 282:1145-1147).

Polynucleotides encoding INTSIG can also be used to create “knockin” humanized animals (pigs) or transgenic animals (mice or rats) to model human disease. With knockin technology, a region of a polynucleotide encoding INTSIG is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress INTSIG, e.g., by secreting INTSIG in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of INTSIG and intracellular signaling molecules. In addition, examples of tissues expressing INTSIG can be found in Table 6 and can also be found in Example XI. Therefore, INTSIG appears to play a role in cell proliferative, autoimmune/inflammatory, neurological, gastrointestinal, reproductive, developmental, and vesicle trafficking disorders. In the treatment of disorders associated with increased INTSIG expression or activity, it is desirable to decrease the expression or activity of INTSIG. In the treatment of disorders associated with decreased INTSIG expression or activity, it is desirable to increase the expression or activity of INTSIG.

Therefore, in one embodiment, INTSIG or a fragment or derivative thereof may be administered to a subject to treat or prevent a disorder associated with decreased expression or activity of INTSIG. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythermia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, exthroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracozporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyosiuis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive supranuclear palsy, corticobasal degeneration, and familial frontotemporal dementia; a gastroitestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis, cancer of the breast, fibrocystic breast disease, galactorrhea, a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumours; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Chiarcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a vesicle trafficking disorder such as cystic fibrosis, glucose-galactose malabsorption syndrome, hypercholesterolemia, diabetes mellitus, diabetes insipidus, hyper- and hypoglycemia, Grave's disease, goiter, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, myasthenia gravis, rheumatoid and osteoarthritis, scleroderma, Chediak-Higasbi and Sjogren's syndromes, systemic lupus erythematosus, toxic shock syndrome, traumatic tissue damage, Williams syndrome, late infantile neuronal ceroid lipofuccinosis, and viral, bacterial, fungal, helminthic, and protozoal infections.

In another embodiment, a vector capable of expressing INTSIG or a fragment or derivative thereof may be administered to a subject to treat or prevent a disorder associated with decreased expression or activity of INTSIG including, but not limited to, those described above.

In a further embodiment, a composition comprising a substantially purified INTSIG in conjunction with a suitable pharmaceutical carrier may be administered to a subject to treat or prevent a disorder associated with decreased expression or activity of INTSIG including, but not limited to, those provided above.

In still another embodiment, an agonist which modulates the activity of INTSIG maybe administered to a subject to treat or prevent a disorder associated with decreased expression or activity of INTSIG including, but not limited to, those listed above.

In a further embodiment, an antagonist of INTSIG may be administered to a subject to treat or prevent a disorder associated with increased expression or activity of INTSIG. Examples of such disorders include, but are not limited to, those cell proliferative, autoimmune/inflammatory, neurological, gastrointestinal, reproductive, developmental, and vesicle trafficking disorders described above. In one aspect, an antibody which specifically binds INTSIG may be used directly as an antagonist or indirectly as a targeting or delivery mechanism for bringing a pharmaceutical agent to cells or tissues which express INTSIG.

In an additional embodiment, a vector expressing the complement of the polynucleotide encoding INTSIG maybe administered to a subject to treat or prevent a disorder associated with increased expression or activity of INTSIG including, but not limited to, those described above.

In other embodiments, any protein, agonist, antagonist, antibody, complementary sequence, or vector embodiments maybe administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of INTSIG may be produced using methods which are generally known in the art. In particular, purified INTSIG may be used to produce antibodies or to screen libraries of pharmaceutical agents to identify those which specifically bind INTSIG. Antibodies to INTSIG may also be generated using methods that-are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others maybe immunized by injection with INTSIG or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, 1 and dinitrophenol. Among adjuvants used in humans, BCG (bacilli Calmette-Guerin) and Corynebacterium parvum are especially preferable.

It is preferred that the oligopeptides, peptides, or fragments used to induce antibodies to INTSIG have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of INTSIG amino acids maybe fused with those of another protein, such as KLH, and antibodies to the chimeric molecule maybe produced.

Monoclonal antibodies to INTSIG may be prepared using any technique which provides for the production of antibody molecules by continuous cell lines in culture. These include, but are not limited to, the hybridoma technique, the human B-cell hybridoma technique, and the EBV-hybridoma technique. (See, e.g., Kohler, G. et al. (1975) Nature 256:495-497; Kozbor, D. et al (1985) J. Immunol. Methods 81:31-42; Cote, R. J. et al. (1983) Proc. Natl. Acad. Sci. USA 80:2026-2030; and Cole, S. P. et al. (1984) Mol. Cell Biol. 62:109-120.)

In addition, techniques developed for the production of “chimeric antibodies,” such as the splicing of mouse antibody genes to human antibody genes to obtain a molecule with appropriate antigen specificity and biological activity, can be used. (See, e.g., Morrison, S. L. et al. (1984) Proc. Natl. Acad. Sci. USA 81:6851-6855; Neuberger, M. S. et al. (1984) Nature 312:604-608; and Takeda, S. et al. (1985) Nature 314:452-454.) Alternatively, techniques described for the production of single chain antibodies may be adapted, using methods known in the art, to produce INTSIG-specific single chain antibodies. Antibodies with related specificity, but of distinct idiotypic composition, may be generated by chain shuffling from random combinatorial imrnunoglobulin libraries. (See, e.g., Burton, D. R. (1991) Proc. Natl. Acad. Sci. USA 88:10134-10137.) Antibodies may also be produced by inducing in vivo production in the lymphocyte population or by screening immunoglobulin libraries or panels of highly specific binding reagents as disclosed in the literature. (See, e.g., Orlandi, R. et al. (1989) Proc. Natl. Acad. Sci. USA 86:3833-3837; Winter, G. et al. (1991) Nature 349:293-299.)

Antibody fragments which contain specific binding sites for INTSIG may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (See, e.g., Huse, W. D. et al. (1989) Science 246:1275-1281.)

Various immunoassays maybe used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between INTSIG and its specific antibody. A two-site, monoclonal-based immunoassay utilizing monoclonal antibodies reactive to two non-interfering INTSIG epitopes is generally used, but a competitive binding assay may also be employed (Pound, supra).

Various methods such as Scatchard analysis in conjunction with radioimmunoassay techniques may be used to assess the affinity of antibodies for INTSIG. Affinity is expressed as an association constant, Kₐ, which is defined as the molar concentration of INTSIG-antibody complex divided by the molar concentrations of free antigen and free antibody under equilibrium conditions. The Kₐ determined for a preparation of polyclonal antibodies, which are heterogeneous in their affinities for multiple INTSIG epitopes, represents the average affinity, or avidity, of the antibodies for INTSIG. The Kₐ determined for a preparation of monoclonal antibodies, which are monospecific for a particular INTSIG epitope, represents a true measure of affinity. High-affinity antibody preparations with Kₐ ranging from about 10⁹ to 10¹² L/mole are preferred for use in immunoassays in which the INTSIG-antibody complex must withstand rigorous manipulations. Low-affinity antibody preparations with Kₐ ranging from about 10⁶ to 10⁷ L/mole are preferred for use in immunopurification and similar procedures which ultimately require dissociation of INTSIG, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. ° For example, a polyclonal antibody preparation containing at least 1-2 mg specific antibody/ml, preferably 5-10 mg specific antibody/ml, is generally employed in procedures requiring precipitation of INTSIG-antibody complexes. Procedures for evaluating antibody specificity, titer, and avidity, and guidelines for antibody quality and usage in various applications, are generally available. (See, e.g., Catty, supra, and Coligan et al. supra.)

In another embodiment of the invention, polynucleotides encoding INTSIG, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding INTSIG. Such technology is well known in the art, and antisense oligonucleotides or larger fragments can be designed from various locations along the coding or control regions of sequences encoding INTSIG. (See, e.g., Agrawal, S., ed. (1996) Antisense Therapeutics, Humana Press Inc., Totawa N.J.)

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (See, e.g., Slater, J. E. et al. (1998) J. Allergy Clin. Immunol. 102(3):469-475; and Scanlon, K. J. et al. (1995) 9(13):1288-1296.) Antisense sequences can also be introduced intracellularly through the use of viral vectors, such as retrovirus and adeno-associated virus vectors. (See, e.g., Miller, A. D. (1990) Blood 76:271; Ausubel, supra; Uckert, W. and W. Walther (1994) Pharmacol. Ther. 63(3):323-347.) Other gene delivery mechanisms include liposome-derived systems, artificial viral envelopes, and other systems known in the art. (See, e.g., Rossi, J. J. (1995) Br. Med. Bull. 51(1):217-225; Boado, R. J. et al (1998) J. Pharm. Sci. 87(11):1308-1315; and Morris, M. C. et al. (1997) Nucleic Acids Res. 25(14):2730-2736.)

In another embodiment of the invention, polynucleotides encoding INTSIG maybe used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasilietisis; and protozoan parasites such as Plasmodium falciparum and Trypanosoma cruzi). In the case where a genetic deficiency in INTSIG expression or regulation causes disease, the expression of INTSIG from an appropriate population of transduced cells may alleviate the clinical manifestations caused by the genetic deficiency.

In a further embodiment of the invention, diseases or disorders caused by deficiencies in INTSIG are treated by constructing mammalian expression vectors encoding INTSIG and introducing these vectors by mechanical means into INTSIG-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J-L. and H. Récipon (1998) Curr. Opin. Biotechnol. 9:445-450).

Expression vectors that may be effective for the expression of INTSIG include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). INTSIG maybe expressed using (i) a constitutively active promoter, (e.g., from cytomegalovirus (CMV), Rous sarcoma virus (RSV), SV40 virus, thymidine kinase (TX), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding INTSIG from a normal individual.

Commercially available liposome transformation kits (e.g., the PERFECT LIPID TRANSPECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to INTSIG expression are treated by constructing a retrovirus vector consisting of (i) the polynucleotide encoding INTSIG under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virot 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding INTSIG to cells which have one or more genetic abnormalities with respect to the expression of INTSIG. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al (1995) Transplantation 27:263-268). Potentially useful adenoviral vectors are described in U.S. Patent No. 5,707,618 to Armentano (“Adenovirus vectors for gene therapy”), hereby incorporated by reference. For adenoviral vectors, see also Antinozzi, P. A. et al. (1999) Annu. Rev. Nutr. 19:511-544 and Verma, I. M. and N. Somia (1997) Nature 18:389:239-242, both incorporated by reference herein.

In another embodiment, a herpes-based, gene therapy delivery system is used to deliver polynucleotides encoding INTSIG to target cells which have one or more genetic abnormalities with respect to the expression of INTSIG. The use of herpes simplex virus (HSV)-based vectors may be especially valuable for introducing INTSIG to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999) J. Virol. 73:519-532 and Xu, H. et al. (1994) Dev. Biol. 163:152-161, hereby incorporated by reference. The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding INTSIG to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for INTSIG into the alphavirus genome in place of the capsid-coding region results in the production of a large number of INTSIG-coding RNAs and the synthesis of high levels of INTSIG in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of INTSIG into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have S been described in the literature. (See, e.g., Gee, J. E. et al. (1994) in Huber, B. E. and B. I. Carr, Molecular and Immunologic Approaches, Futura Publishing, Mt. Kisco N.Y., pp. 163-177.) A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding INTSIG.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules maybe generated by in vitro and in vivo transcription of DNA molecules encoding INTSIG. Such DNA sequences may be incorporated into a wide variety of vectors with suitable RNA polymerase promoters such as 17 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding INTSIG. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression Thus, in the treatment of disorders associated with increased INTSIG expression or activity, a compound which specifically inhibits expression of the polynucleotide encoding INTSIG may be therapeutically useful, and in the treatment of disorders associated with decreased INTSIG expression or activity, a compound which specifically promotes expression of the polynucleotide encoding INTSIG may be therapeutically useful.

At least one, and up to a plurality, of test compounds maybe screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding INTSIG is exposed to at least one test compound thus obtained. The sample may comprise, for example, an intact or permeabilized cell, or an in vitro cell-free or reconstituted biochemical system. Alterations in the expression of a polynucleotide encoding INTSIG are assayed by any method commonly known in the art. Typically, the expression of a specific nucleotide is detected by hybridization with a probe having a nucleotide sequence complementary to the sequence of the polynucleotide encoding INTSIG. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nat. Biotechnol. 15:462-466.)

Any of the therapeutic methods described above maybe applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of INTSIG, antibodies to INTSIG, and mimetics, agonists, antagonists, or inhibitors of INTSIG.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising INTSIG or fragments thereof. For example, liposome preparations containing a cell-impermeable macromolecule may promote cell fusion and intracellular delivery of the macromolecule. Alternatively, INTSIG or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration Such information can then be used to determine useful doses and routes for administration in humans.

A therapeutically effective dose refers to that amount of active ingredient, for example INTSIG or fragments thereof, antibodies of INTSIG, and agonists, antagonists or inhibitors of INTSIG, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD50 (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment Dosage and administration are adjusted to provide sufficient levels of the active moiety or to maintain the desired effect. Factors which may be taken into account include the severity of the-disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 ηg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind INTSIG maybe used for the diagnosis of disorders characterized by expression of INTSIG, or in assays to monitor patients being treated with INTSIG or agonists, antagonists, or inhibitors of INTSIG. Antibodies useful for diagnostic purposes may be prepared in the same manner as described above for therapeutics. Diagnostic assays for INTSIG include methods which utilize the antibody and a label to detect INTSIG in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring INTSIG, including ELISAs, RIAs, and FACS, are known in the art and provide a basis for diagnosing altered or abnormal levels of INTSIG expression. Normal or standard values for INTSIG expression are established by combining body fluids or cell extracts taken from normal mammalian subjects, for example, human subjects, with antibodies to INTSIG under conditions suitable for complex formation. The amount of standard complex formation may be quantitated by various methods, such as photometric means. Quantities of INTSIG expressed in subject, control and disease samples from biopsied tissues are compared with the standard values. Deviation between standard and subject values establishes the parameters for diagnosing disease.

In another embodiment of the invention, polynucleotides encoding INTSIG maybe used for diagnostic purposes. The polynucleotides which may be used include oligonucleotides, complementary RNA and DNA molecules, and PNAs. The polynucleotides maybe used to detect and quantify gene expression in biopsied tissues in which expression of INTSIG may be correlated with disease. The diagnostic assay may be used to determine absence, presence, and excess expression of INTSIG, and to monitor regulation of INTSIG levels during therapeutic intervention.

In one aspect, hybridization with PCR probes which are capable of detecting polynucleotides, including genomic sequences, encoding INTSIG or closely related molecules may be used to identify nucleic acid sequences which encode INTSIG. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding INTSIG, allelic variants, or related sequences.

Probes may also be used for the detection of related sequences, and may have at least 50% sequence identity to any of the INTSIG encoding sequences. The hybridization probes of the subject invention may be DNA or RNA and may be derived from the sequence of SEQ ID NO:21-40 or from genomic sequences including promoters, enhancers, and introns of the DUSIG gene.

Means for producing specific hybridization probes for polynucleotides encoding INTSIG include the cloning of polynucleotides encoding INTSIG or INTSIG derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding INTSIG may be used for the diagnosis of disorders associated with expression of INTSIG. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal noctual hemoglobinuria, polycythemia vera, psoriasis, primary thrombocythemia, and cancers including adenocarcinoma, leukemia, lymphoma, melanoma, myeloma, sarcoma, teratocarcinoma, and, in particular, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythernatosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorders of the central nervous system including Down syndrome, cerebral palsy, neuroskeletal disorders, autonomic nervous system disorders, cranial nerve disorders, spinal cord diseases, muscular dystrophy and other neuromuscular disorders, peripheral nervous system disorders, dermatomyositis and polymyositis, inherited, metabolic, endocrine, and toxic myopathies, myasthenia gravis, periodic paralysis, mental disorders including mood, anxiety, and schizophrenic disorders, seasonal affective disorder (SAD), akathesia, amnesia, catatonia, diabetic neuropathy, tardive dyskinesia, dystonias, paranoid psychoses, postherpetic neuralgia, Tourette's disorder, progressive suprarnuclear palsy, corticobasal degeneration, and familial frontotemporal dementia; a gastrointestinal disorder such as dysphagia, peptic esophagitis, esophageal spasm, esophageal stricture, esophageal carcinoma, dyspepsia, indigestion, gastritis, gastric carcinoma, anorexia, nausea, emesis, gastroparesis, antral or pyloric edema, abdominal angina, pyrosis, gastroenteritis, intestinal obstruction, infections of the intestinal tract, peptic ulcer, cholelithiasis, cholecystitis, cholestasis, pancreatitis, pancreatic carcinoma, biliary tract disease, hepatitis, hyperbilirubinemia, cirrhosis, passive congestion of the liver, hepatoma, infectious colitis, ulcerative colitis, ulcerative proctitis, Crohn's disease, Whipple's disease, Mallory-Weiss syndrome, colonic carcinoma, colonic obstruction, irritable bowel syndrome, short bowel syndrome, diarrhea, constipation, gastrointestinal hemorrhage, acquired immunodeficiency syndrome (AIDS) enteropathy, jaundice, hepatic encephalopathy, hepatorenal syndrome, hepatic steatosis, hemochromatosis, Wilson's disease, alpha₁-antitrypsin deficiency, Reye's syndrome, primary sclerosing cholangitis, liver infarction, portal vein obstruction and thrombosis, centrilobular necrosis, peliosis hepatis, hepatic vein thrombosis, veno-occlusive disease, preeclampsia, eclampsia, acute fatty liver of pregnancy, intrahepatic cholestasis of pregnancy, and hepatic tumors including nodular hyperplasias, adenomas, and carcinomas; a reproductive disorder such as a disorder of prolactin production, infertility, including tubal disease, ovulatory defects, endometriosis, a disruption of the estrous cycle, a disruption of the menstrual cycle, polycystic ovary syndrome, ovarian hyperstimulation syndrome, an endometrial or ovarian tumor, a uterine fibroid, autoimmune disorders, ectopic pregnancy, teratogenesis, cancer of the breast, fibrocystic breast disease, galactorrhea, a disruption of spermatogenesis, abnormal sperm physiology, cancer of the testis, cancer of the prostate, benign prostatic hyperplasia, prostatitis, Peyronie's disease, impotence, carcinoma of the male breast, gynecomastia, hypergonadotropic and hypogonadotropic hypogonadism, pseudohermaphroditism, azoospermia, premature ovarian failure, acrosin deficiency, delayed puperty, retrograde ejaculation and anejaculation, haemangioblastomas, cystsphaeochromocytomas, paraganglioma, cystadenomas of the epididymis, and endolymphatic sac tumours; a developmental disorder such as renal tubular acidosis, anemia, Cusbing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; and a vesicle trafficking disorder such as cystic fibrosis, glucose-galactose malabsorption syndrome, hypercholesterolemia, diabetes mellitus, diabetes insipidus, hyper- and hypoglycemia, Grave's disease, goiter, Cushing's disease, and Addison's disease, gastrointestinal disorders including ulcerative colitis, gastric and duodenal ulcers, other conditions associated with abnormal vesicle trafficking, including acquired immunodeficiency syndrome (AIDS), allergies including hay fever, asthma, and urticaria (hives), autoimmune hemolytic anemia, proliferative glomerulonephritis, inflammatory bowel disease, multiple sclerosis, myasthenia gravis, rheumatoid and osteoarthritis, scleroderma, Chediak-Higashi and Sjogren's syndromes, systemic lupus erythematosus, toxic shock syndrome, traumatic tissue damage, Williams syndrome, late infantile neuronal ceroid lipoficcinosis, and viral, bacterial, fungal, helminthic, and protozoal infections. Polynucleotides encoding INTSIG may be used in Southern or northern analysis, dot blot, or other membrane-based technologies; in PCR technologies; in dipstick, pin, and multiformat ELISA-like assays; and in microarrays utilizing fluids or tissues from patients to detect altered INTSIG expression. Such qualitative or quantitative methods are well known in the art.

In a particular aspect, polynucleotides encoding INTSIG maybe used in assays that detect the presence of associated disorders, particularly those mentioned above. Polynucleotides complementary to sequences encoding INTSIG maybe labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding INTSIG in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of INTSIG, a normal or standard profile for expression is established. This maybe accomplished by combining body fluids or cell extracts taken from normal subjects, either animal or human, with a sequence, or a fragment thereof, encoding INTSIG, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) inbiopsied tissue from an individual may indicate a predisposition for the development of the disease, or may provide a means for detecting the disease-prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding INTSIG may involve the use of PCR. These oligomers may be chemically synthesized, generated enzymatically, or produced in vitro. Oligomers will preferably contain a fragment of a polynucleotide encoding INTSIG, or a fragment of a polynucleotide complementary to the polynucleotide encoding INTSIG, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding INTSIG maybe used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding INTSIG are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations. (Taylor, J. G. et al. (2001) Trends Mol Med. 7:507-512; Kwok, P.-Y. and Z. Giu (1999) Mol. Med. Today 5:538-543; Nowotny, P. et al. (2001) Curr. Opin. Neurobiol. 11:637-641.) Methods which may also be used to quantify the expression of INTSIG include radiolabeling or biotinylating nucleotides, coamplification of a control nucleic acid, and interpolating results from standard curves. (See, e.g., Melby, P. C. et al. (1993) J. Immunol. Methods 159:235-244; Duplaa, C. et al. (1993) Anal. Biochem. 212:229-236.) The speed of quantitation of multiple samples maybe accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, INTSIG, fragments of INTSIG, or antibodies specific for INTSIG may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time. (See Seilhamer et al., “Comparative Gene Transcript Analysis,” U.S. Pat. No. 5,840,484, expressly incorporated by reference herein.) Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression iii vivo, as in the case of a tissue or biopsy sample, or in vitro, as in the case of a cell line.

Transcript images which profile: the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different: compounds. While the assignment of gene function to elements of a toxicant signature aids in interpretation of toxicity mechanisms, knowledge of gene function is not necessary for the statistical matching of signatures which leads to prediction of toxicity. (See, for example, Press Release 00-02 from the National Institute of Environmental Health Sciences, released Feb. 29, 2000, available at http://www.niehs.nih.gov/oc/news/toxchip.htm.) Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data maybe obtained for definitive protein identification.

A proteomic profile may also be generated using antibodies specific for INTSIG to quantity the levels of INTSIG expression. In one embodiment, the antibodies are used as elements on a microarray, and protein expression levels are quantified by exposing the microarray to the sample and detecting the levels of protein bound to each array element (Lueling, A. et al (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures maybe useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays maybe prepared, used, and analyzed using methods known in the art. (See, e.g., Brennan, T. M. et al (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; and Heller, M. J. et al. (1997) U.S. Pat. No. 5,605,662.) Various types of microarrays are well known and thoroughly described in DNA Microarrays: A Practical Approach, M. Schena, ed. (1999) Oxford University Press, London.

In another embodiment of the invention, nucleic acid sequences encoding INTSIG may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g.,.human artificial chromosomes (HACs), yeast artificial chromosomes (YACs), bacterial artificial chromosomes (BACs), bacterial P1 constructions, or single chromosome cDNA libraries. (See, e.g., Harrington, J. J. et al. (1997) Nat. Genet. 15:345-355; Price, C. M. (1993) Blood Rev. 7:127-134; and Trask, B. J. (1991) Trends Genet. 7:149-154.) Once mapped, the nucleic acid sequences may be used to develop genetic linkage maps, for example, which correlate the inheritance of a disease state with the inheritance of a particular chromosome region or restriction fragment length polymorphism (RFLP). (See, for example, Lander, E. S. and D. Botstein (1986) Proc. Natl. Acad. Sci. USA 83:7353-7357.)

Fluorescent in situ hybridization (FISH) may be correlated with other physical and genetic map data. (See, e.g., Heinz-Ulrich, et al. (1995) in Meyers, supra, pp. 965-968.) Examples of genetic map data can be found in various scientific journals or at the Online Mendelian Inheritance in Man (OMIM) World Wide Web site. Correlation between the location of the gene encoding INTSIG on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation. (See, e.g., Gatti, R. A. et al. (1988) Nature 336:577-580.) The nucleotide sequence of the instant invention may also be used to detect differences in the chromosomal location due to translocation, inversion, etc., among normal, carrier, or affected individuals.

In another embodiment of the invention, INTSIG, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between INTSIG and the agent being tested may be measured.

Another technique for drug screening provides for high throughput screening of compounds having suitable binding affinity to the protein of interest. (See, e.g., Geysen, et al. (1984) PCT application WO84/03564.) In this method, large numbers of different small test compounds are synthesized on a solid substrate. The test compounds are reacted with INTSIG, or fragments thereof, and washed. Bound INTSIG is then detected by methods well known in the art. Purified INTSIG can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding INTSIG specifically compete with a test compound for binding INTSIG. In this manner, antibodies can be-used to detect the presence of any peptide which shares one or more antigenic determinants with INTSIG.

In additional embodiments, the nucleotide sequences which encode INTSIG may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, including U.S. Ser. No. 60/297,010, U.S. Ser. No. 60/301,871, U.S. Ser No. 60/299,998, U.S. Ser No. 60/303,403, U.S. Ser No. 60/298,706, U.S. Ser No. 60/303,349, U.S. Ser No. 60/300,377, and U.S. Ser No. 60/351,927 are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the NIZAP vector system (Stratagene) or SUPERSCRIPT plasmid system (Invitrogen), using the recommended procedures or similar methods known in the art. (See, e.g., Ausubel, 1997, supra, units 5.1-6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CIAB column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pR4R (cyte Genomics), or pINCY (bncyte Genomics), or derivatives thereof. Recombinant plasmids were transformed into competent E. coli cells including XL1-Blue, XL1-BlueMRP, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4 ° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (reviewed in Ausubel, 1997, supra, unit 7.7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTBOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. R et al. (2001) Nucleic Acids Res. 29:41-43); and HMM-based protein domain databases such as SMART (Schultz et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families. See, for example, Eddy, S. R. (1996) Curr. Opin. Struct. Biol 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Thred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Pull length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (Hitachi Software Engineering, South San Francisco Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:21-40. Fragments from about 20 to about 4000 nucleotides which are useful in hybridization and amplification technologies are described in Table 4, column 2.

IV. Identification and Editing of Coding Sequences from Genomic DNA

Putative intracellular signaling molecules were initially identified by running the Genscan gene identification program against public genomic sequence databases (e.g., gbpri and gbhtg). Genscan is a general-purpose gene identification program which analyzes genomic DNA sequences from a variety of organisms (See Burge, C. and S. Karlin (1997) J. Mol. Biol. 268:78-94, and Burge, C. and S. Karlin (1998) Curr. Opin. Struct. Biol. 8:346-354). The program concatenates predicted exons to form an assembled cDNA sequence extending from a methionine to a stop codon. The output of Genscan is a PASTA database of polynucleotide and polypeptide sequences. The maximum range of sequence for Genscan to analyze at once was set to 30 kb. To determine which of these Genscan predicted cDNA sequences encode intracellular signaling molecules, the encoded polypeptides were analyzed by querying against PFAM models for intracellular signaling molecules. Potential intracellular signaling molecules were also identified by homology to Incyte cDNA sequences that had been annotated as intracellular signaling molecules. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example m were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of INTSIG Encoding Polynucleotides

The sequences which were used to assemble SEQ ID NO:21-40 were compared with sequences from the Incyte LIFESEQ database and public domain databases using BLAST and other implementations of the Smith-Waterman algorithm. Sequences from these databases that matched SEQ ID NO:21-40 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound. (See, e.g., Sambrook, supra, ch. 7; Ausubel (1995) supra, ch. 4 and 16.)

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding INTSIG are analyzed with respect to the tissue sources from which they were derived. For example, some full length sequences are assembled, at least in part, with overlapping Incyte cDNA sequences (see Example E). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding INTSIG. cDNA sequences and cDNA library/tissue information are found in the LIFESEQ GOLD database (Incyte Genomics, Palo Alto Calif.).

VIII. Extension of INTSIG Encoding Polynucleotides

Full length polynucleotides are produced by extension of an appropriate fragment of the full length molecule using oligonucleotide primers designed from this fragment One primer was synthesized to initiate 5′ extension of the known fragment, and the other primer was synthesized to initiate 3′ extension of the known fragment The initial primers were designed using OLIGO 4.06 software (National Biosciences), or another appropriate program, to be about 22 to 30 nucleotides in length, to have a GC content of about 50% or more, and to anneal to the target sequence at temperatures of about 68 ° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68 OC, 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1× TF, and 0.5 μl of undiluted PCR product into each well of an opaque fluorimeter plate (Corning Costar, Acton Mass.), allowing the DNA to bind to the reagent. The plate was scanned in a Fuoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACB (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in INTSIG Encoding Polynucleotides

Common DNA sequence variants known as single nucleotide polymorphisms (SNPs) were identified in SEQ ID NO:21-40 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:21-40 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1 x saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (inkjet printing, See, e.g., Baldeschweiler, supra.), mechanical microspotting technologies, and derivatives thereof. The substrate in each of the aforementioned technologies should be uniform and solid with a non-porous surface (Schena (1999), supra). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements. (See, e.g., Schena, M. et al. (1995) Science 270:467-470; Shalon, D. et al. (1996) Genome Res. 6:639-645; Marshall, A. and J. Hodgson (1998) Nat. Biotechnol. 16:27-31.)

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may-be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (CLONTECH Laboratories, Inc. (CLONTECH), Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Micro arrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45OC in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMr R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RnI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

Expression

SEQ ID NO:33 showed differential expression in association with inflammatory responses, as determined by microarray analysis. The expression of SEQ ID NO:33 was increased by at least two-fold in human peripheral blood mononuclear cells (PBMCs) treated with PMA (a broad activator of protein kinase C-dependent pathways) and with ionomycin (a calcium ionophore that permits the entry of calcium in the cell) relative to untreated PBMCs. In PBMCs, the combination of PMA and ionomycin mimics the secondary signaling events elicited during activation of lymphocytes, NK cells, and monocytes. Therefore, in an embodiment, SEQ ID NO:33 can be used in diagnostic assays and/or for monitoring treatment of immune response disorders, and related diseases and conditions.

In addition, SEQ ID NO:38 and SEQ ID NO:39 showed differential expression in association with Alzheimer's disease, as determined by microarray analysis. The expression of SEQ ID NO:38 and SEQ ID NO:39 was decreased at least two-fold in amygdala tissue from human brains with mild or severe Alzheimer's disease as compared to amygdala tissue from normal brains. Therefore, in an embodiment, SEQ ID NO:38 and SEQ ID NO:39 can be used in diagnostic assays and/or for monitoring treatment of Alzheimer's disease.

In addition, SEQ ID NO:22 showed differential expression in association with lung squamous carcinoma tissues versus normal lung tissues, as determined by microarray analysis. The expression of SEQ ID NO:22 was decreased at least two-fold in lung squamous carcinoma tissues as compared to grossly uninvolved normal lung tissue from the same donor. Thus, in an embodiment, SEQ ID NO:22 can be used in diagnostic assays and/or for monitoring treatment of lung cancer.

XII. Complementary Polynucleotides

Sequences complementary to the INTSIG-encoding sequences, or any parts thereof, are used to detect, decrease, or inhibit expression of naturally occurring INTSIG. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of INTSIG. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the INTSIG-encoding transcript.

XIII. Expression of INTSIG

Expression and purification of INTSIG is achieved using bacterial or virus-based expression systems. For expression of INTSIG in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express INTSIG upon induction with isopropyl beta-D-thiogalactopyranoside (IPTG). Expression of INTSIG in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding INTSIG by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus. (See Engelhard, E. K. et al. (1994) Proc. Natl. Acad. Sci. USA 91:3224-3227; Sandig, V. et al. (1996) Hum. Gene Ther. 7:1937-1945.)

In most expression systems, INTSIG is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from INTSIG at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel (1995, supra, ch. 10 and 16). Pured INTSIG obtained by these methods can be used directly in the assays shown in Examples XVII, XVI, and XIX, where applicable.

XIV. Functional Assays

INTSIG function is assessed by expressing the sequences encoding INTSIG at physiologically elevated levels in mammalian cell culture systems. cDNA is subdloned into a mammalian expression vector containing a strong promoter that drives high levels of cDNA expression. Vectors of choice include PCMV SPORT plasmid (Invitrogen, Carlsbad CA) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synathesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometery, Oxford, New York N.Y.

The influence of INTSIG on gene expression can be assessed using highly purified populations of cells transfected with sequences encoding INTSIG and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding INTSIG and other genes of interest can be analyzed by northern analysis or microarray techniques.

XV. Production of INTSIG Specific Antibodies

INTSIG substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the INTSIG amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or inhydrophilic regions are well described in the art. (See, e.g., Ausubel, 1995, supra, ch. 11.) Typically, oligopeptides of about 15 residues in length are synthesized using an ABI 43 IA peptide synthesizer (Applied Biosystems) using FMOC chemistry and coupled to KIL (Sigma-Aldrich, St. Louis Mo.) by reaction with N-maleimidobenzoyl-N-hydroxysuccinimide ester (MBS) to increase immunogenicity. (See, e.g., Ausubel, 1995, supra.) Rabbits are immunized with the oligopeptide-KLH complex in complete Preund's adjuvant. Resulting antisera are tested for antipeptide and anti-SIG activity by, for example, binding the peptide or INTSIG to a substrate, blocking with 1% BSA, reacting with rabbit antisera, washing, and reacting with radio-iodinated goat anti-rabbit IgG.

XVI. Purification of Naturally Occurring INTSIG Using Specific Antibodies

Naturally occurring or recombinant INTSIG is substantially purified by immunoaffinity chromatography using antibodies specific for INTSIG. An immunoaffinity column is constructed by covalently coupling anti-INTSIG antibody to an activated chromatographic resin, such as CNBr-activated SEPHAROSE (Amersham Biosciences). After the coupling, the resin is blocked and washed according to the manufacturer's instructions.

Media containing INTSIG are passed over the immunoaffinity column, and the column is washed under conditions that allow the preferential absorbance of INTSIG (e.g., high ionic strength buffers in the presence of detergent). The column is eluted under conditions that disrupt antibody/INTSIG binding (e.g., a buffer of pH 2 to pH 3, or a high concentration of a chaotrope, such as urea or thiocyanate ion), and INTSIG is collected.

XVII. Identification of Molecules Which Interact with INTSIG

INTSIG, or biologically active fragments thereof, are labeled with ¹²⁵I Bolton-Hunter reagent. (See, e.g., Bolton, A. E. and W. M. Hunter (1973) Biochem. J. 133:529-539.) Candidate molecules previously arrayed in the wells of a multi-well plate are incubated with the labeled INTSIG, washed, and any wells with labeled INTSIG complex are assayed. Data obtained using different concentrations of INTSIG are used to calculate values for the number, affinity, and association of INTSIG with the candidate molecules.

Alternatively, molecules interacting with INTSIG are analyzed using the yeast two-hybrid system as described in Fields, S. and O. Song (1989) Nature 340:245-246, or using commercially available kits based on the two-hybrid system, such as the MATCHMAKER system (Clontech).

INTSIG may also be used in the PATHCALLJNG process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of INTSIG Activity

INTSIG activity is associated with its ability to form protein-protein complexes and is measured by its ability to regulate growth characteristics of NIH3T3 mouse fibroblast cells. A cDNA encoding INTSIG is subdloned into an appropriate eukaryotic expression vector. This vector is transfected into NIH3T3 cells using methods known in the art. Transfected cells are compared with non-transfected cells for the following quantifiable properties: growth in culture to high density, reduced attachment of cells to the substrate, altered cell morphology, and ability to induce tumors when injected into immunodeficient mice. The activity of INTSIG is proportional to the extent of increased growth or frequency of altered cell morphology in NIH3T3 cells transfected with INTSIG.

Alternatively, INTSIG activity is measured by binding of INTSIG to radiolabeled formin polypeptides containing the proline-rich region that specifically binds to SH3 containing proteins (Chan, D. C. et al. (1996) EMBO J. 15:1045-1054). Samples of INTSIG are run on SDS-PAGE gels, and transferred onto nitrocellulose by electroblotting. The blots are blocked for 1 hr at room temperature in TBST (137 mM NaCl, 2.7 mM KCl, 25 mM Tris (pH 8.0) and 0.1% Tween-20) containing non-fat dry milk. Blots are then incubated with TBST containing the radioactive formin polypeptide for 4 hrs to overnight After washing the blots four times with TBST, the blots are exposed to autoradiographic film. Radioactivity is quantitated by cutting out the radioactive spots and counting them in a radioisotope counter. The amount of radioactivity recovered is proportional to the activity of INTSIG in the assay.

Alternatively, PDE activity of INTSIG is measured by monitoring the conversion of a cyclic nucleotide (either cAMEP or cGMP) to its nucleotide monophosphate. The use of tritium-containing substrates such as ³H-cAMP and ³H-cGMP, and 5′ nucleotidase from snake venom, allows the PDE reaction to be followed using a scintillation counter. cAMP-specific PDE activity of INTSIG is assayed by measuring the conversion of ³H-cAMP to ³H-adenosine in the presence of INTSIG and 5′ nucleotidase. A one-step assay is run using a 100 μL reaction containing 50 mM Tris-HCl pH 7.5, 10 mM MgCl₂, 0.1 unit 5′ nucleotidase (from Crotalus atrox venom), 0.0062-0.1 μM ³H-cAMP, and various concentrations of cAMP (0.0062-3 mM). The reaction is started by the addition of 25 μl of diluted enzyme supernatant. Reactions are run directly in mini Poly-Q scintillation vials (Beckman Instruments, Pullerton Calif.). Assays are incubated at 37° C. for a time period that would give less than 15% cAMP hydrolysis to avoid non-linearity associated with product ibikbition. The reaction is stopped by the addition of 1 ml of Dowex (Dow Chemical, Midland Mich.) AG1×8 (Cl form) resin (1:3 slurry). Three ml of scintillation fluid are added, and the vials are mixed. The resin in the vials is allowed to settle for one hour before counting. Soluble radioactivity associated with ³H-adenosine is quantitated using a beta scintillation counter. The amount of radioactivity recovered is proportional to the cAMP-specific PDE activity of INTSIG in the reaction. For inhibitor or agonist studies, reactions are carried out under the conditions described above, with the addition of 1% DMSO, 50 nM cAMP, and various concentrations of the inhibitor or agonist. Control reactions are carried out with all reagents except for the enzyme aliquot.

In an alternative assay, cGMP-specific PDE activity of INTSIG is assayed by measuring the conversion of ³H-cGMP to ³H-guanosine in the presence of INTSIG and 5′ nucleotidase. A one-step assay is run using a 100 μl reaction containing 50 mM Tris-HCl pH 7.5, 10 mM MgCl₂, 0.1 unit 5′ nucleotidase (from Crotalus atrox venom), and 0.0064-2.0 μM ³H-cGMP. The reaction is started by the addition of 25 μl of diluted enzyme supernatant. Reactions are run directly in mini Poly-Q scintillation vials (Beckman Instruments). Assays are incubated at 37° C. for a time period that would yield less than 15% cGMP hydrolysis in order to avoid non-linearity associated with product inhibition. The reaction is stopped by the addition of 1 ml of Dowex (Dow Chemical, Midland Mich.) AG1×8 (Cl form) resin (1:3 slurry). Three nl of scintillation fluid are added, and the vials are mixed. The resin in the vials is allowed to settle for one hour before counting. Soluble radioactivity associated with ³H-guanosine is quantitated using a beta scintillation counter. The amount of radioactivity recovered is proportional to the cGMP-specific PDE activity of INTSIG in the reaction. For inhibitor or agonist studies, reactions are carried out under the conditions described above, with the addition of 1% DMSO, 50 nM cGMT, and various concentrations of the inhibitor or agonist Control reactions are carried out with all reagents except for the enzyme aliquot.

Alternatively, INTSIG protein kinase activity is measured by quantifying the phosphorylation of an appropriate substrate in the presence of gamma-labeled ³²P-ATP. INTSIG is incubated with the substrate, ³²P-ATP, and an appropriate kinase buffer. The ³²P incorporated into the product is separated from free ³²P-ATP by electrophoresis, and the incorporated ³²P is quantified using a beta radioisotope counter. The amount of incorporated ³²P is proportional to the protein kinase activity of INTSIG in the assay. A determination of the specific amino acid residue phosphorylated by protein kinase activity is made by phosphoamino acid analysis of the hydrolyzed protein.

Alternatively, an assay for INTSIG protein phosphatase activity measures the hydrolysis of para-nitrophenyl phosphate (PNPP). INTSIG is incubated together with PNPP in HEPES buffer pH 7.5, in the presence of 0.1% β-mercaptoetianol at 37° C. for 60 min. The reaction is stopped by the addition of 6 ml of 10 N NaOH, and the increase in light absorbance of the reaction mixture at 410 nm resulting from the hydrolysis of PNPP is measured using a spectrophotometer. The increase in light absorbance is proportional to the activity of INTSIG in the assay (Diamond, R. H. et al. (1994) Mol. Cell Biol. 14:3752-3762).

Alternatively, adenylyl cyclase activity of INTSIG is demonstrated by the ability to convert ATP to cAMP (Mittal, C. K. (1986) Meth. Enzymol. 132:422-428). In this assay INTSIG is incubated with the substrate [α-³²P]ATP, following which the excess substrate is separated from the product cyclic [³²P] AMP. INTSIG activity is determined in 12×75 mm disposable culture tubes containing 5 μl of 0.6 M Tris-HCl, pH 7.5, 5 μl of 0.2.M MgCl₂, 5 μl of 150 mM creatine phosphate containing 3 units of creatine phosphokinase, 5 μl of 4.0 mM 1-methyl-3-isobutylxanthine, 5 μl of 20 mM cAMP, 5 μl 20 mM dithiothreitol, 5 μl of 10, mM ATP, 10 μl [α³²P]ATP (2-4×10⁶ cpm), and water in a total volume of 100 μl. The reaction mixture is prewarmed to 30° C. The reaction is initiated by adding INTSIG to the prewarmed reaction mixture. After 10-15 minutes of incubation at 30° C., the reaction is terminated by adding 25 μl of 30% ice-cold trichloroacetic acid (TCA). Zero-time incubations and reactions incubated in the absence of INTSIG are used as negative controls. Products are separated by ion exchange chromatography, and cyclic [³²P] AMP is quantified using a β-radioisotope counter. The INTSIG activity is proportional to the amount of cyclic [³²P] AMP formed in the reaction.

An alternative assay measures INTSIG-mediated G-protein signaling activity by monitoring the mobilization of Ca²⁺ as an indicator of the signal transduction pathway stimulation. (See, e.g., Grynldewicz, G. et al. (1985) J. Biol. Chem. 260:3440; McColl, S. et al. (1993) J. Immunol. 150:4550-4555; and Aussel supra). The assay requires preloading neutrophils or T cells with a fluorescent dye such as FURA-2 or BCECF (Universal Imaging Corp, Westchester Pa.) whose emission characteristics are altered by Ca²⁺ binding. When the cells are exposed to one or more activating stimuli artificially (e.g., anti-CD3 antibody ligation of the T cell receptor) or physiologically (e.g., by alogeneic stimulation), Ca²⁺ flux takes place. This flux can be observed and quantified by assaying the cells in a fluorometer or fluorescent activated cell sorter. Measurements of Ca²⁺ flux are compared between cells in their normal state and those transfected with INTSIG. Increased Ca²⁺ mobilization attributable to increased INTSIG concentration is proportional to INTSIG activity.

Alternatively, GTP-binding activity of INTSIG is determined in an assay that measures the binding of INTSIG to [α-³²P]-labeled GTP. Purified INTSIG is first blotted onto filters and rinsed in a suitable buffer. The filters are then incubated in buffer containing radiolabeled [α-³²P]-GTP. The filters are washed in buffer to remove unbound GDP and counted in a radioisotope counter. Non-specific binding is determined in an assay that contains a 100-fold excess of unlabeled GTP. The amount of specific binding is proportional to the activity of INTSIG.

Alternatively, GTPase activity of INTSIG is determined in an assay that measures the conversion of [α-³²P]-GTP to [α-³²P]-GDP. INTSIG is incubated with [α-³²P]-GTP in buffer for an appropriate period of time, and the reaction is terminated by heating or acid precipitation followed by centrifugation. An aliquot of the supernatant is subjected to polyacrylamide gel electrophoresis (PAGE) to separate GDP and GTP together with unlabeled standards. The GDP spot is cut out and counted in a radioisotope counter. The amount of radioactivity recovered in GDP is proportional to the GTPase activity of INTSIG.

Alternatively, INTSIG activity is measured by quantifying the amount of a non-hydrolyzable GTP analogue, GTPγS, bound over a 10 minute incubation period. Varying amounts of INTSIG are incubated at 30° C. in 50 mM Tris buffer, pH 7.5, containing 1 mM dithiothreitol, 1 mM EDTA and 1 μM [³⁵S]GTPγS. Samples are passed through nitrocellulose filters and washed twice with a buffer consisting of 50 mM Tris-HCl, pH 7.8, 1 mM NaN₃, 10 mM MgCl₂, 1 mM EDTA, 0.5 mM dithiothreitol, 0.01 mM PMSF, and 200 mM NaCl. The filter-bound counts are measured by liquid scintillation to quantify the amount of bound [³⁵S]GTPγS. INTSIG activity may also be measured as the amount of GTP hydrolysed over a 10 minute incubation period at 37° C. INTSIG is incubated in 50 mM Tris-HCl buffer, pH 7.8, containing 1 mM dithiothreitol, 2 mM EDTA, 10 μM [α-³²P]GTP, and 1 μM H-rab protein GTPase activity is initiated by adding MgCl₂ to a final concentration of 10 mM. Samples are removed at various time points, mixed with an equal volume of ice-cold 0.5 mM EDTA, and frozen. Aliquots are spotted onto polyethyleneimine-cellulose thin layer chromatography plates, which are developed in 1 M LiCl, dried, and autoradiographed. The signal detected is proportional to INTSIG activity.

Alternatively, INTSIG activity may be demonstrated as the ability to interact with its associated LMW GTPase in an in vitro binding assay. The candidate LMW GTPases are expressed as fusion proteins with glutathione S-transferase (GST), and purified by affinity chromatography on glutathione-Sepharose. The LMW GTpases are loaded with GDP by incubating 20 mM Tris buffer, pH 8.0, containing 100 mM NaCl, 2 mM EDTA, 5 mM MgCl₂, 0.2 mM DTT, 100 μM AMP-PNP and 10 μM GDP at 30° C. for 20 minutes. INTSIG is expressed as a FLAG fusion protein in a baculovirus system. Extracts of these baculovirus cells containing INTSIG-FLAG fusion proteins are precleared with GST beads, then incubated with GST-GTPase fusion proteins. The complexes formed are precipitated by glutathione-Sepharose and separated by SDS-polyacrylamide gel electrophoresis. The separated proteins are blotted onto nitrocellulose membranes and probed with commercially available anti-FLAG antibodies. INTSIG activity is proportional to the amount of INTSIG-FLAG fusion protein detected in the complex.

Another alternative assay to detect INTSIG activity is the use of a yeast two-hybrid system (Zalcman, G. et al. (1996) J. Biol. Chem. 271:30366-30374). Specifically, a plasmid such as pGAD1318 which may contain the coding region of INTSIG can be used to transform reporter L40 yeast cells which contain the reporter genes LacZ and HIS3 downstream from the binding sequences for LexA. These yeast cells have been previously transformed with a pLexA-Rab6-GDP (mouse) plasmid or with a plasmid which contains pLexA-lamin C. The pLEXA-lamin C cells serve as a negative control. The transformed cells are plated on ahistidine-free medium and incubated at 30° C. for 3 days. His⁺ colonies are subsequently patched on selective plates and assayed for β-galactosidase activity by a filter assay. INTSIG binding with Rab6-GDP is indicated by positive His⁺/lacZ⁺ activity for the cells transformed with the plasmid containing the mouse Rab6-GDP and negative His⁺/lacZ⁺ activity for those transformed with the plasmid containing lamin C.

Alternatively, INTSIG activity is measured by binding of INTSIG to a substrate which recognizes WD-40 repeats, such as ElonginB, by coimmunoprecipitation (Kamura, T. et al. (1998) Genes Dev. 12:3872-3881). Briefly, epitope tagged substrate and INTSIG are mixed and immunoprecipitated with commercial antibody against the substrate tag. The reaction solution is run on SDS-PAGE and the presence of INTSIG visualized using an antibody to the INTSIG tag. Substrate binding is proportional to INTSIG activity.

Alternatively, INTSIG activity is measured by its inclusion in coated vesicles. INTSIG can be expressed by transforming a mammalian cell line such as COS7, HeLa, or CHO with a eukaryotic expression vector encoding INTSIG. Eukaryotic expression vectors are commercially available, and the techniques to introduce them into cells are well known to those skilled in the art. A small amount of a second plasmid, which expresses any one of a number of marker genes, such as β-galactosidase, is co-transformed into the cells in order to allow rapid identification of those cells which have taken up and expressed the foreign DNA. The cells are incubated for 48-72 hours after transformation under conditions appropriate for the cell line to allow expression and accumulation of INTSIG and β-galactosidase.

In the alternative, INTSIG activity is measured by its ability to alter vesicle trafficking pathways. Vesicle trafficking in cells transformed with INTSIG is examined using fluorescence microscopy. Antibodies specific for vesicle coat proteins or typical vesicle trafficking substrates such as transferrin or the mannose-6-phosphate receptor are commercially available. Various cellular components such as ER, Golgi bodies, peroxisomes, endosomes, lysosomes, and the plasmalemma are examined. Alterations in the numbers and locations of vesicles in cells transformed with INTSIG as compared to control cells are characteristic of INTSIG activity. Transformed cells are collected and cell lysates are assayed for vesicle formation. A non-hydrolyzable form of GTP, GTPγS, and an ATP regenerating system are added to the lysate and the mixture is incubated at 37° C. for 10 minutes. Under these conditions, over 90% of the vesicles remain coated (Orci, L et al (1989) Cell 56:357-368). Transport vesicles are salt-released from the Golgi membranes, loaded under a sucrose gradient, centrifuged, and fractions are collected and analyzed by SDS-PAGE. Co-localization of INTSIG with clathrin or COP coatamer is indicative of INTSIG activity in vesicle formation. The contribution of INTSIG in vesicle formation can be confirmed by incubating lysates with antibodies specific for INTSIG prior to GTPγS addition. The antibody will bind to INTSIG and interfere with its activity, thus preventing vesicle formation.

XIX. SNX Binding Activity

SNX proteins participate in protein trafficking through their interaction with various growth factors, protein receptors, and membrane associations. The binding of SNX to membranes or receptors, such as tyrosine kinases, can be evaluated through coexpression assays (Haft, C. R., et al. (1998) Mol. Cell Biol. 18: 7278-7287). For example, Myc-tagged SNX15 can be prepared in COS7 cells (American Type Culture Collection) and coupled with expression vectors encoding receptors for growth factors of interest (Phillips, S. A., et al. (2001) J. Biol. Chem. 276: 5074-5084). Following cotransfection of Myc-SNX15 with the receptor, immunoblotting with an anti-Myc antibody of cell extracts can be used to detect the distribution of receptors and epitope-tagged sorting nexins.

Various modifications and variations of the described compositions, methods, and systems of the invention will be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| Incyte | | | Poly- | Incyte |
|---|---|---|---|---|
| Project | Polypeptide | Incyte | nucleotide | Polynucleotide |
| ID | SEQ ID NO: | Polypeptide ID | SEQ ID NO: | ID |
| 7488243 | 1 | 7488243CD1 | 21 | 7488243CB1 |
| 1966295 | 2 | 1966295CD1 | 22 | 1966295CB1 |
| 113399 | 3 | 113399CD1 | 23 | 113399CB1 |
| 3418524 | 4 | 3418524CD1 | 24 | 3418524CB1 |
| 7490407 | 5 | 7490407CD1 | 25 | 7490407CB1 |
| 700648 | 6 | 700648CD1 | 26 | 700648CB1 |
| 2744459 | 7 | 2744459CD1 | 27 | 2744459CB1 |
| 60204026 | 8 | 60204026CD1 | 28 | 60204026CB1 |
| 7473835 | 9 | 7473835CD1 | 29 | 7473835CB1 |
| 8186336 | 10 | 8186336CD1 | 30 | 8186336CB1 |
| 7493330 | 11 | 7493330CD1 | 31 | 7493330CB1 |
| 7487969 | 12 | 7487969CD1 | 32 | 7487969CB1 |
| 2655990 | 13 | 2655990CD1 | 33 | 2655990CB1 |
| 71768694 | 14 | 71768694CD1 | 34 | 71768694CB1 |
| 5079019 | 15 | 5079019CD1 | 35 | 5079019CB1 |
| 894500 | 16 | 894500CD1 | 36 | 894500CB1 |
| 7497866 | 17 | 7497866CD1 | 37 | 7497866CB1 |
| 832718 | 18 | 832718CD1 | 38 | 832718CB1 |
| 7497717 | 19 | 7497717CD1 | 39 | 7497717CB1 |
| 7506420 | 20 | 7506420CD1 | 40 | 7506420CB1 |

TABLE 2

| Polypeptide | Incyte | GenBank ID NO: | | |
|---|---|---|---|---|
| SEQ | Polypeptide | or PROTEOME | Probability | |
| ID NO: | ID | ID NO: | Score | Annotation |
| 1 | 7488243CD1 | g3184512 | 2.3E−30 | [Gallus gallus] GTPase cRac1B |
| | | | | Malosio, M. L. et al. (1997) Differential expression of distinct members of Rho |
| | | | | family GTP-binding proteins during neuronal development: identification of |
| | | | | Rac1B, a new neural-specific member of the family. |
| | | | | J. Neurosci. 17: 6717-6728. |
| 2 | 1966295CD1 | g13569476 | 7.8E−40 | [Mus musculus] immunity-associated nucleotide 4 |
| | | | | Daheron, L. et al. (2001) Molecular cloning of Ian4: a BCR/ABL-induced gene |
| | | | | that encodes an outer membrane mitochondrial protein with GTP-binding activity. |
| | | | | Nucleic Acids Res. 29: 1308-1316. |
| 3 | 113399CD1 | g15042691 | 1.0E−141 | sorting nexin 18 [Homo sapiens] |
| 4 | 3418524CD1 | g13445784 | 0.0 | [Mus musculus] Rab6-interacting protein 2 isoform A |
| 5 | 7490407CD1 | g36036 | 1.9E−65 | [Homo sapiens] GTPase |
| | | | | Vincent, S. et al. (1992) Growth-regulated expression of rhoG, a new member of |
| | | | | the ras homolog gene family. Mol. Cell. Biol. 12: 3138-3148. |
| 6 | 700648CD1 | g485144 | 6.7E−37 | [Caenorhabditis elegans] similar to guanine-nucleotide releasing factors including |
| | | | | BCR |
| | | | | Wilson, R. et al. (1994) 2.2 Mb of contiguous nucleotide sequence from |
| | | | | chromosome III of C. elegans. Nature 368: 32-38. |
| 7 | 2744459CD1 | g2088556 | 2.8E−247 | [Rattus norvegicus] regulator of G-protein signalling 14 |
| | | | | Snow, B. E. et al. (1997) Molecular cloning and expression analysis of rat Rgs12 |
| | | | | and Rgs14 Biochem. Biophys. Res. Commun. 233: 770-777. |
| 8 | 60204026CD1 | g5762305 | 0.0 | [Mus musculus] COP1 protein |
| | | | | Wang, H. et al. (1999) Evidence for functional conservation of a mammalian |
| | | | | homologue of the light-responsive plant protein COP1 Curr. Biol. 9: 711-714. |
| 9 | 7473835CD1 | g4589375 | 2.3E−197 | [Homo sapiens] Gab2 |
| | | | | Nishida, K. et al. (1999) Gab-family adapter proteins act downstream of cytokine |
| | | | | and growth factor receptors and T- and B-cell antigen receptors. Blood 93: 1809-1816. |
| 10 | 8186336CD1 | g5050926 | 1.0E−39 | [Homo sapiens] dJ100N22.1 (novel EGF-like domain containing protein) |
| 10 | 8186336CD1 | g10998440 | 4.8E−33 | [Mus musculus] EGF-related protein SCUBE1 |
| | | | | Grimmond, S. et al. (2000) Cloning, Mapping, and Expression Analysis of a Gene |
| | | | | Encoding a Novel Mammalian EGF-Related Protein (SCUBE1). Genomics 70: 74-81. |
| 11 | 7493330CD1 | g3347953 | 0.0 | [Rattus norvegicus] cytosolic sorting protein PACS-1a |
| | | | | Wan, L., et al. (1998) PACS-1 defines a novel gene family of cytosolic sorting |
| | | | | proteins required for trans-Golgi network localization. Cell 94, 205-216. |
| 12 | 7487969CD1 | g1924944 | 2.5E−79 | [Homo sapiens] SNAP23B protein |
| | | | | Mollinedo, F. and Lazo, P. A. (1997) Identification of two isoforms of the vesicle- |
| | | | | membrane fusion protein SNAP-23 in human neutrophils and HL-60 cells. |
| | | | | Biochem. Biophys. Res. Commun. 231, 808-812. |
| 13 | 2655990CD1 | g312212 | 1.3E−20 | [Homo sapiens] rhoGAP protein |
| | | | | Lancaster, C. A. et al. (1994) Characterization of rhoGAP. A GTPase-activating |
| | | | | protein for rho-related small GTPases. J. Biol. Chem. 269: 1137-1142. |
| 14 | 71768694CD1 | g18700711 | 0.0 | [Mus musculus] Dual-specificity Rho- and Arf-GTPase activating protein 1 |
| 15 | 5079019CD1 | g179810 | 1.3E−29 | [Homo sapiens] calmodulin |
| | | | | Wawrzynczak, E. J. and R. N. Perham (1984) Biochem. Int. 9: 177-185. |
| 15 | 5079019CD1 | g19919856 | 2.0E−74 | [Homo Sapiens] calmodulin-like protein |
| 16 | 894500CD1 | g2443369 | 8.4E−211 | [Homo sapiens] Nck-associated protein NAP5 |
| | | | | Matuoka, K. et al. (1997) Biochem. Biophys. Res. Commun. 239: 488-492. |
| 17 | 7497866CD1 | g4102877 | 8.8E−287 | [Mus musculus] Shc binding protein |
| | | | | Schmandt, R. et al. (1999) Oncogene 18: 1867-1879. |
| 18 | 832718CD1 | g11863685 | 0.0 | [Mus musculus] neurobeachin |
| | | | | Wang, X., et al. (2000) Neurobeachin: A protein kinase A-anchoring, |
| | | | | beige/Chediak-higashi protein homolog implicated in neuronal membrane traffic. |
| | | | | J Neurosci 20, 8551-8565. |
| 19 | 7497717CD1 | g11863684 | 0.0 | [Mus musculus] neurobeachin |
| | | | | Wang et al., supra |
| 20 | 7506420CD1 | g4102877 | 7.9E−263 | [Mus musculus] Shc binding protein |
| | | | | Schmandt, R., et al. (1999) Oncogene 18: 1867-1879 |
| 20 | 7506420CD1 | 430252\| | 6.9E−264 | [Mus musculus] Shc SH2-domain binding protein 1 (protein expressed in |
| | | Shcbp1 | | activated lymphocytes), binds to the Shc (Shc1) SH2 domain in a phosphotyrosine- |
| | | | | independent manner, expressed only in actively dividing cells, may function in |
| | | | | cell cycle signaling pathways. |

TABLE 3

| | | Ami- | | | | |
|---|---|---|---|---|---|---|
| | | no | | | | |
| SEQ | Incyte | Acid | Potential | Potential | | |
| ID | Polypeptide | Resi- | Phosphorylation | Glycosylation | | Analytical Methods |
| NO: | ID | dues | Sites | Sites | Signature Sequences, Domains and Motifs | and Databases |
| 1 | 7488243CD1 | 144 | T35 T59 T84 | N39 N57 | Transforming protein P21 RAS signature: PR00449: | BLIMPS_PRINTS |
| | | | | | I4-T25, S27-N43, V44-P66, P103-V116 | |
| | | | | | RAS TRANSFORMING PROTEIN | BLAST_DOMO |
| | | | | | DM00006\|P15154\|1-156: M1-L123 | |
| | | | | | DM00006\|Q03206\|1-156: M1-R125 | |
| | | | | | DM00006\|P34145\|1-156: M1-E118 | |
| | | | | | DM00006\|P34144\|1-156: M1-E118 | |
| | | | | | ATP/GTP-binding site motif A (P-loop): G10-T17 | MOTIFS |
| 2 | 1966295CD1 | 665 | S21 S174 S292 | N170 N639 | Transmembrane domain: K79-T107 | TMAP |
| | | | S299 S308 S381 | | N-terminus is non-cytosolic. | |
| | | | S479 S508 S519 | | IMMUNE ASSOCIATED PROTEIN PD119787: | BLAST_PRODOM |
| | | | S628 S651 T107 | | V529-K658, P92-N203, I320-H430 | |
| | | | T113 T134 T237 | | ATP/GTP-binding site motif A (P-loop): G17-S24, | MOTIFS |
| | | | T337 T364 T404 | | G254-S261, G445-S452 | |
| | | | T449 T465 T538 | | | |
| | | | T565 | | | |
| 3 | 113399CD1 | 574 | S44 S54 S108 | N144 | PX domain: P227-L336 | HMMER_PFAM |
| | | | S172 S177 S192 | | SH3 domain: L3-V59 | HMMER_PFAM |
| | | | S233 S251 S324 | | SH3 domain signature PR00452: L3-S13, E17-E32, | BLIMPS_PRINTS |
| | | | S334 S385 S499 | | L35-S44, E47-V59 | |
| | | | T33 T125 T295 | | | |
| | | | T380 T446 T567 | | | |
| | | | Y274 | | | |
| 4 | 3418524CD1 | 972 | S4 S21 S27 S50 | N36 N822 N954 | PROTEIN COILED COIL CHAIN MYOSIN | BLAST_PRODOM |
| | | | S75 S142 S185 | | REPEAT HEAVY ATP BINDING FILAMENT | |
| | | | S251 S308 S322 | | HEPTAD PD000002: E570-M810 | |
| | | | S348 S374 S392 | | CHROMOSOME PROTEIN COILED COIL | BLAST_PRODOM |
| | | | S415 S448 S476 | | HEPTAD REPEAT PATTERN ATP BINDING I | |
| | | | S596 S669 S674 | | MAJOR PD075049: L414-L675 | |
| | | | S694 S734 S751 | | TRICHOHYALIN DM03839 | BLAST_DOMO |
| | | | S807 S846 S857 | | \|P22793\|921-1475: E290-E845 | |
| | | | S892 S905 S906 | | \|P37709\|632-1103: L464-R935 | |
| | | | T84 T150 T287 | | Leucine zipper pattern: L844-L865 | MOTIFS |
| | | | T297 T326 T333 | | | |
| | | | T382 T480 T487 | | | |
| | | | T511 T522 T609 | | | |
| | | | T616 T617 T910 | | | |
| | | | Y650 | | | |
| 5 | 7490407CD1 | 189 | S120 T3 T35 T44 | N39 | signal_cleavage: M1-P29 | SPSCAN |
| | | | T114 T124 T183 | | | |
| | | | | | Ras family: K5-L189 | HMMER_PFAM |
| | | | | | GTP-binding nuclear prot BL01115: | BLIMPS_BLOCKS |
| | | | | | I4-D47, T83-K126 | |
| | | | | | Transforming protein P21 RAS signature PR00449: | BLIMPS_PRINTS |
| | | | | | I4-T25, V27-Q43, T44-D66, P105-L118, | |
| | | | | | Y153-V175 | |
| | | | | | RAS TRANSFORMING PROTEIN DM00006 | BLAST_DOMO |
| | | | | | \|P35238\|1-156: M1-E155 | |
| | | | | | \|P15154\|1-156: M1-E155 | |
| | | | | | \|I45715\|1-156: M1-E155 | |
| | | | | | \|O03206\|1-156: M1-E155 | |
| | | | | | ATP/GTP-binding site motif A (P-loop): G10-T17 | MOTIFS |
| 6 | 700648CD1 | 1935 | S10 S14 S56 S121 | N186 | RhoGEF domain: V942-K1125 | HMMER_PFAM |
| | | | S122 S123 S147 | | | |
| | | | S149 S172 S204 | | TRANSMEMBRANE domain: L1402-G1430 | TMAP |
| | | | S262 S266 S278 | | N-terminus is non-cytosolic. | |
| | | | S335 S365 S422 | | PROTEIN FACTOR GUANINENUCLEOTIDE | BLAST_PRODOM |
| | | | S439 S504 S509 | | RELEASING NUCLEOTIDE GUANINE | |
| | | | S514 S595 S605 | | EXCHANGE PROTOONCOGENE BINDING SH3 | |
| | | | S636 S645 S699 | | PD000777: V942-K1125 | |
| | | | S704 S732 S757 | | BCR PROTEIN DM08397\|P11274\|435-971: | BLAST_DOMO |
| | | | S786 S794 S802 | | E923-K1175; \|A49307\|26-564: P914-V1127 | |
| | | | S825 S874 S952 | | VAV; KINASE; ZINC; SH2; DM08580 | BLAST_DOMO |
| | | | S956 S980 S1064 | | \|P52735\|1-491: D937-S1198 | |
| | | | S1129 S1196 S1198 | | \|P15498\|1-483: E911-S1198 | |
| | | | S1203 S1211 S1250 | | | |
| | | | S1275 S1292 S1347 | | | |
| | | | S1350 S1380 S1404 | | | |
| | | | S1419 S1442 S1445 | | | |
| | | | S1487 S1509 S1511 | | | |
| | | | S1543 S1553 S1554 | | | |
| | | | S1578 S1589 S1597 | | | |
| | | | S1630 S1637 S1731 | | | |
| | | | S1900 S1916 T61 | | | |
| | | | T270 T314 T405 | | | |
| | | | T412 T420 T618 | | | |
| | | | T945 T1099 T1191 | | | |
| | | | T1240 T1306 | | | |
| | | | T1313 T1449 | | | |
| | | | T1470 T1552 | | | |
| | | | T1748 T1870 | | | |
| 7 | 2744459CD1 | 567 | S20 S198 S243 | N93 N265 N560 | signal_cleavage: M1-S51 | SPSCAN |
| | | | S260 S279 S280 | | | |
| | | | S284 S338 S390 | | LGN motif, putative GEF specific for | HMMER_PFAM |
| | | | S411 S540 S561 | | G-alpha: I499-L521 | |
| | | | T121 T207 T292 | | Raf-like Ras-binding domain: | HMMER_PFAM |
| | | | T323 T381 T394 | | K302-R373, T375-L445 | |
| | | | T491 T496 | | Regulator of G protein signaling domain: S67-L184 | HMMER_PFAM |
| | | | | | Regulator of G protein s PF00615: | BLIMPS_PFAM |
| | | | | | F84-C100, I162-V175 | |
| | | | | | RGS12 REGULATOR OF GPROTEIN SIGNALING | BLAST_PRODOM |
| | | | | | SIGNAL TRANSDUCTION INHIBITOR RGS14 | |
| | | | | | ALTERNATIVE PD016903: K352-P477, S488-P546, | |
| | | | | | A318-Q351 | |
| | | | | | REGULATOR OF SIGNALING GPROTEIN | BLAST_PRODOM |
| | | | | | SIGNAL TRANSDUCTION INHIBITOR RGS12 | |
| | | | | | PROTEIN RGS14 PD013247: L185-Q351 | |
| | | | | | REGULATOR OF GPROTEIN SIGNALING RGS14 | BLAST_PRODOM |
| | | | | | SIGNAL TRANSDUCTION INHIBITOR | |
| | | | | | RAP1/RAP2 INTERACTING PD033865: M1-A65 | |
| | | | | | RECEPTOR KINASE GPROTEIN SIGNAL | BLAST_PRODOM |
| | | | | | TRANSDUCTION INHIBITOR REGULATOR OF | |
| | | | | | SIGNALING G PD001580: S67-L184 | |
| | | | | | RGS DOMAIN DM01609\|P49798\|20-186: E58-Y180; | BLAST_DOMO |
| | | | | | \|P49808\|1-167: G34-R181; \|P41220\|42-207: | |
| | | | | | P53-E182; \|P49796\|353-518: P55-L193 | |
| 8 | 60204026CD1 | 731 | S110 S126 S163 | N350 N419 N661 | signal_cleavage: M1-A53 | SPSCAN |
| | | | S287 S324 S355 | | WD domain, G-beta repeat: R505-S542, P463-D499, | HMMER_PFAM |
| | | | S373 S387 S404 | | P591-N626, S548-D584, C632-Y668, Y413-E449, | |
| | | | S425 S489 S531 | | D690-E729 | |
| | | | S566 S609 S635 | | Zinc finger, C3HC4 type (RING finger): C136-C173 | HMMER_PFAM |
| | | | S650 S685 T228 | | | |
| | | | T343 T361 T442 | | Beta G-protein (transducin) signature | BLIMPS_PRINTS |
| | | | T543 T677 T682 | | PR00319: I613-V627, S650-Y667 | |
| | | | T724 | | COP1 PROTEIN ZINCFINGER REPEAT | BLAST_PRODOM |
| | | | | | REGULATORY NUCLEAR WD FUSCA FUS1 | |
| | | | | | PHOTOMORPHOGENESIS PD020219: L633-L730; | |
| | | | | | PD024847: R359-M467; PD154832: T543-V594 | |
| | | | | | MSI1; CORONIN; YMR131C; YDR128W; | BLAST_DOMO |
| | | | | | DM00614\|P43254\|445-498: V495-V549 | |
| | | | | | Trp-Asp (WD) repeats signature: I613-V627 | MOTIFS |
| | | | | | Zinc finger, C3HC4 type (RING finger), signature: | MOTIFS |
| | | | | | C151-I160 | |
| 9 | 7473835CD1 | 654 | S4 S11 S29 S42 | N303 N404 N500 | PH domain: M1-G107 | HMMER_PFAM |
| | | | S81 S130 S190 | | KIAA0571 PROTEIN GRB2ASSOCIATED | BLAST_PRODOM |
| | | | S276 S347 S366 | | BINDER1 GAB1 PD018409: I105-S612 | |
| | | | S394 S411 S502 | | | |
| | | | S526 S615 T27 T91 | | | |
| | | | T216 T256 T274 | | | |
| | | | T380 T557 T559 | | | |
| | | | T635 Y86 | | | |
| 10 | 8186336CD1 | 80 | S56 T53 | | signal_cleavage: M1-L22 | SPSCAN |
| | | | | | Signal Peptide: M1-G20 | HMMER |
| | | | | | Signal Peptide: M1-G24 | HMMER |
| | | | | | Calcium-binding EGF-like BL01187: | BLIMPS_BLOCKS |
| | | | | | S31-D42, Y50-Y65 | |
| | | | | | Type II EGF-like signature PR00010: | BLIMPS_PRINTS |
| | | | | | K55-Y65, E68-D74 | |
| | | | | | EGF-like domain signature 2: C59-C72 | MOTIFS |
| 11 | 7493330CD1 | 963 | S150 S268 S278 | N202 N276 N527 | Transmembrane domain: D652-V669 | TMAP |
| | | | S287 S381 S411 | N570 N617 N648 | N-terminus is cytosolic. | |
| | | | S430 S451 S485 | | PROTEIN CYTOSOLIC SORTING PUTATIVE | BLAST_PRADOM |
| | | | S486 S497 S523 | | KRUEPPEL TARGET GENE PACS1A PACS1B | |
| | | | S529 S531 S595 | | PD152707: R94-I543 | |
| | | | S683 S694 S844 | | PD156370: G755-L956, P544-L723 | |
| | | | S892 S916 T124 | | | |
| | | | T314 T319 T394 | | | |
| | | | T453 T461 T504 | | | |
| | | | T511 T731 T763 | | | |
| | | | T848 Y277 Y656 | | | |
| | | | Y742 | | | |
| 12 | 7487969CD1 | 175 | S5 S23 T24 | N3 N102 | SNAP-25 family domain: S5-R144 | HMMER_PFAM |
| | | | T43 T69 T72 T135 | | PROTEIN SYNAPTOSOMAL ASSOCIATED | BLAST_PRODOM |
| | | | T140 | | SYNAPTOSOME SNAP23 NEURONE SNAP25 | |
| | | | | | VESICLEMEMBRANE FUSION | |
| | | | | | SYNAPTOSOMEASSOCIATED | |
| | | | | | PD004321: E7-E98 | |
| | | | | | PD168856: D93-S158 | |
| 13 | 2655990CD1 | 731 | S26 S68 S84 S308 | N8 N306 N320 | RhoGAP domain: P101-L250 | HMMER_PFAM |
| | | | S377 S383 S400 | N394 N523 N563 | | |
| | | | S411 S463 S479 | | PROTEIN GTPASE DOMAIN ACTIVATION: | BLIMPS_PRODOM |
| | | | S508 S528 S547 | | PD00930: P101-A126, L201-M241 | |
| | | | S577 S595 S622 | | P value <0.0011 | |
| | | | S714 T20 T264 | | PROTEIN GTPASE DOMAIN SH2 ACTIVATION | BLAST_PRODOM |
| | | | T288 T398 T434 | | ZINC 3-KINASE SH3 PHOSPHATIDYLINOSITOL | |
| | | | T492 T493 T495 | | REGULATORY: | |
| | | | T565 T696 | | PD000780: L100-S249 | |
| | | | | | PH DOMAIN: | BLAST_DOMO |
| | | | | | DM00470\|P55194\|113-387: P103-N272 | |
| | | | | | DM00470\|S54307\|1621-1845: M64-I270 | |
| | | | | | DM00470\|P34588\|1-285: S74-F268 | |
| | | | | | DM00470\|A38218\|1145-1413: K82-C273 | |
| 14 | 71768694CD1 | 727 | S57 S111 S146 | N563 | Signal Peptide: M1-G25 | HMMER |
| | | | S159 S189 S282 | | PH domain: S420-H521, L23-G99 | HMMER_PFAM |
| | | | S420 S460 S461 | | RhoGAP domain: P116-E267 | HMMER_PFAM |
| | | | S502 S533 S574 | | GTPase-activator protein: PF00620B: F168-D184 | BLIMPS_PFAM |
| | | | S624 S663 T73 | | PROTEIN GTPASE DOMAIN ACTIVATION: | BLIMPS_PRODOM |
| | | | T175 T303 T331 | | PD00930: P116-G141, L219-V259 | |
| | | | T339 T504 T610 | | PROTEIN GTPASE DOMAIN SH2 ACTIVATION | BLAST_PRODOM |
| | | | T615 | | ZINC 3KINASE SH3 PHOSPHATIDYLINOSITOL | |
| | | | | | REGULATORY: | |
| | | | | | PD000780: I115-Q261 | |
| | | | | | PH DOMAIN: | BLAST_DOMO |
| | | | | | DM00470\|S54307\|1621-1845: I94-Q261 | |
| | | | | | DM00470\|P46941\|504-803: I115-G264 | |
| | | | | | DM00470\|P34588\|1-285: I115-V259 | |
| | | | | | DM00470\|A38218\|1145-1413: I115-D263 | |
| 15 | 5079019CD1 | 159 | S154 T47 T109 | | Signal peptide: M1-S49 | SPSCAN |
| | | | | | EF hand: E125-E153, E89-A117, E14-L42 | HMMER-PFAM |
| | | | | | EF-hand calcium-binding domain: | BLIMPS-BLOCKS |
| | | | | | D134-F146 | |
| | | | | | Calcium binding protein, muscle, myosin, light chain, | BLAST-PRODOM |
| | | | | | calmodulin | |
| | | | | | PD000012: A83-M150 | |
| | | | | | EF-hand calcium-binding domain: | MOTIFS |
| | | | | | D134-F146 | |
| 16 | 894500CD1 | 1356 | S163 S188 S1040 | N13 N207 N227 | Acidic serine cluster repeat: DM04746\|S57757\|1-646: | BLAST-DOMO |
| | | | S308 S346 S1069 | N521 N614 N635 | L285-E901 | |
| | | | S381 S425 S1172 | N676 N744 | | |
| | | | S468 S477 S1191 | N1067 N1160 | | |
| | | | S499 S508 S1203 | | | |
| | | | S564 S592 S1218 | | | |
| | | | S602 S616 S1242 | | | |
| | | | S646 S678 S1247 | | | |
| | | | S685 S722 S1303 | | | |
| | | | S745 S775 S1305 | | | |
| | | | S837 S863 S1307 | | | |
| | | | S871 S876 S1337 | | | |
| | | | S949 S950 S977 | | | |
| | | | T15 T28 T38 T89 | | | |
| | | | S23 S8 S58 S90 | | | |
| | | | S1346 T120 T167 | | | |
| | | | T339 T382 T1018 | | | |
| | | | T392 T493 T1163 | | | |
| | | | T630 T637 T1225 | | | |
| | | | T820 T845 T870 | | | |
| | | | T885 T899 T922 | | | |
| | | | T958 T992 | | | |
| 17 | 7497866CD1 | 672 | S42 S47 S208 S252 | N279 | | |
| | | | S257 S273 S275 | | | |
| | | | S281 S290 S396 | | | |
| | | | S634 T131 T349 | | | |
| | | | T422 T427 T455 | | | |
| | | | T470 T473 T501 | | | |
| | | | T573 T593 T623 | | | |
| | | | T639 Y524 | | | |
| 18 | 832718CD1 | 2937 | S3 S87 S147 S168 | N363 N465 N515 | Beige/BEACH domain: | HMMER_PFAM |
| | | | S282 S304 S352 | N982 N1143 | T2256-E2509, A2531-R2554 | |
| | | | S420 S461 S517 | N1454 N1463 | WD domain, G-beta repeat: | HMMER_PFAM |
| | | | S561 S674 S680 | N1497 N1808 | L2846-Q2881, Q2887-N2923, | |
| | | | S757 S1016 S1032 | N1851 N2203 | L2703-Y2743, P2763-T2799, | |
| | | | S1043 S1096 S1203 | N2254 N2650 | V2663-S2697, M356-S394 | |
| | | | S1221 S1223 S1230 | N2756 N2839 | | |
| | | | | N2853 | | |
| | | | S1250 S1264 | | Trp-Asp (WD) repeat BL00678: S2732-W2742 | BLIMPS_BLOCKS |
| | | | S1275 S1364 S1367 | | F10F2.1 PROTEIN | BLAST_PRODOM |
| | | | S1415 S1456 S1503 | | PD185994: I91-E971, | |
| | | | | | T1840-T2144, T1328-Q1651 | |
| | | | | | PD185145: A2616-Y2937 | |
| | | | S1687 S1905 S2080 | | PROTEIN TRANSPORT FAN FACTOR | BLAST_PRODOM |
| | | | S2187 S2242 S2265 | | ASSOCIATED WITH NSMASE ACTIVATION | |
| | | | S2390 S2394 S2501 | | REPEAT WD | |
| | | | S2692 S2745 S2855 | | PD007848: G2230-T2597, K2165-R2247 | |
| | | | T153 T191 T285 | | | |
| | | | T415 T634 T647 | | | |
| | | | T664 T713 T813 | | | |
| | | | T838 T1133 T1214 | | | |
| | | | T1242 T1266 | | | |
| | | | T1297 T1417 | | | |
| | | | T1521 T1591 | | | |
| | | | T1610 T1621 | | | |
| | | | T1629 T1665 | | | |
| | | | T1695 T1746 | | | |
| | | | T1802 T1831 | | | |
| | | | T1855 T2018 | | | |
| | | | T2059 T2073 | | | |
| | | | T2083 T2144 | | | |
| | | | T2195 T2216 | | | |
| | | | T2245 T2256 | | | |
| | | | T2280 T2295 | | | |
| | | | T2334 T2700 | | | |
| | | | T2768 T2870 Y345 | | | |
| | | | Y919 Y2287 | | | |
| 19 | 7497717CD1 | 2969 | S3 S87 S147 S168 | N363 N465 N515 | Beige/BEACH domain: | HMMER_PFAM |
| | | | S282 S304 S352 | N982 N1143 | T2288-E2541, A2563-R2586 | |
| | | | S420 S461 S517 | N1454 N1463 | WD domain, G-beta repeat: | HMMER_PFAM |
| | | | S561 S674 S680 | N1497 N1840 | L2878-Q2913, Q2919-N2955, | |
| | | | S757 S1016 S1032 | N1883 N2235 | L2735-Y2775, P2795-T2831, | |
| | | | S1043 S1096 | N2286 N2682 | V2695-S2729, M356-S394 | |
| | | | S1203 S1221 S1223 | N2788 N2871 | Trp-Asp (WD) repeat BL00678: | BLIMPS_BLOCKS |
| | | | S1230 | N2885 | S2764-W2774 | |
| | | | S1250 S1264 S1275 | | F10F2.1 PROTEIN | BLAST_PRODOM |
| | | | S1364 S1367 S1415 | | PD185994: I91-E971, | |
| | | | S1456 S1503 | | T1872-T2176, T1328-P1587 | |
| | | | | | PD185145: A2648-Y2969 | |
| | | | S1719 S1937 S2112 | | PROTEIN TRANSPORT FAN FACTOR | BLAST_PRODOM |
| | | | S2219 S2274 S2297 | | ASSOCIATED WITH NSMASE ACTIVATION | |
| | | | S2422 S2426 S2533 | | REPEAT WD | |
| | | | S2724 S2777 S2887 | | PD007848: G2262-T2629, K2197-R2279 | |
| | | | T153 T191 T285 | | | |
| | | | T415 T634 T647 | | | |
| | | | T664 T713 T813 | | | |
| | | | T838 T1133 T1214 | | | |
| | | | T1242 T1266 | | | |
| | | | T1297 T1417 | | | |
| | | | T1521 T1591 | | | |
| | | | T1653 T1661 | | | |
| | | | T1697 T1727 | | | |
| | | | T1778 T1834 | | | |
| | | | T1863 T1887 | | | |
| | | | T2050 T2091 | | | |
| | | | T2105 T2115 | | | |
| | | | T2176 T2227 | | | |
| | | | T2248 T2277 | | | |
| | | | T2288 T2312 | | | |
| | | | T2327 T2366 | | | |
| | | | T2732 T2800 | | | |
| | | | T2902 Y345 Y919 | | | |
| | | | Y2319 | | | |
| 20 | 7506420CD1 | 616 | S152 S196 S201 | N223 | | |
| | | | S217 S219 S225 | | | |
| | | | S234 S340 S578 | | | |
| | | | T75 T293 T366 | | | |
| | | | T371 T399 T414 | | | |
| | | | T417 T445 T517 | | | |
| | | | T537 T567 T583 | | | |
| | | | Y468 | | | |

TABLE 4

| Polynucleotide | |
|---|---|
| SEQ ID NO:/ | |
| Incyte ID/Sequence | |
| Length | Sequence Fragments |
| 21/7488243CB1/901 | 1-901, 160-593, 160-594 |
| 22/1966295CB1/4064 | 1-244, 1-507, 31-570, 42-282, 50-299, 51-322, 115-643, 120-732, 178-488, 184-786, 456-1120, 460-1112, 583-818, |
| | 583-1139, 600-1166, 614-879, 668-1198, 695-1301, 1030-1233, 1121-2321, 1225-1688, 1229-1688, 1255-1688, |
| | 1273-1688, 1313-1461, 1443-1684, 1624-1883, 1656-1846, 1718-2242, 1814-2268, 1846-2103, 1862-2524, 1930-2521, |
| | 2092-2392, 2224-2466, 2224-2829, 2252-2907, 2258-2481, 2381-2525, 2515-2778, 2528-2791, 2608-3086, |
| | 2639-2944, 2639-3136, 2707-2946, 2707-2996, 2727-3082, 2727-3149, 2819-3127, 2829-3122, 2850-3132, 2850-3326, |
| | 2880-3145, 2925-3125, 2925-3126, 2965-3258, 3061-3252, 3081-3316, 3081-3636, 3108-3310, 3136-3286, |
| | 3159-3414, 3168-3439, 3181-3382, 3181-3383, 3181-3408, 3181-3747, 3218-3496, 3292-3467, 3295-3745, 3314-3450, |
| | 3333-3906, 3335-3511, 3336-3625, 3348-3644, 3360-4042, 3368-4040, 3415-4029, 3416-3954, 3425-4064, |
| | 3435-3711, 3450-4038, 3460-4062, 3468-4040, 3522-3750, 353 1-3802, 3540-3776, 3551-3803, 3584-3828, 3592-3776, |
| | 3597-3815, 3624-3776, 3629-3761, 3630-4049, 3766-4030, 3767-4022 |
| 23/113399CB1/ | 1-272, 1-552, 1-699, 6-152, 14-206, 265-699, 273-771, 338-1014, 339-1233, 346-1026, 347-959, 395-841, 421-889, |
| 3407 | 472-984, 482-885, 486-884, 535-1159, 601-1178, 606-1172, 612-941, 667-1027, 682-1281, 688-1272, 709-1334, |
| | 836-1339, 870-1466, 899-1149, 920-1369, 928-1524, 939-1570, 964-1533, 1025-1602, 1037-1319, 1037-1480, 1038-1536, |
| | 1115-1296, 1152-1696, 1166-1434, 1166-1542, 1166-1821, 1355-1707, 1355-1968, 1366-1921, 1374-1808, |
| | 1423-1731, 1428-1675, 1562-2185, 1631-1902, 1631-2086, 1631-2120, 1631-2150, 1702-1930, 1702-1932, 1789-2053, |
| | 1839-2357, 1892-2409, 2022-2542, 2028-2228, 2028-2248, 2028-2406, 2028-2482, 2028-2547, 2028-2576, |
| | 2028-2629, 2107-2571, 2146-2392, 2164-2416, 2227-2496, 2227-2498, 2227-2593, 2260-2492, 2261-2790, 2273-2711, |
| | 2285-2711, 2285-2714, 2285-2846, 2307-2870, 2325-2962, 2367-2595, 2439-2697, 2465-2676, 2546-2851, |
| | 2566-2778, 2566-3097, 2579-2833, 2597-2885, 2598-2898, 2616-2901, 2619-2919, 2643-2866, 2645-2922, 2653-2897, |
| | 2661-2909, 2691-3023, 2726-3017, 2727-2951, 2727-2986, 2727-3230, 2794-3054, 2794-3307, 2794-3374, |
| | 2794-3385, 2817-3081, 2845-3130, 2868-3096, 2913-3193, 2987-3223, 3024-3298, 3180-3385, |
| | 3180-3386, 3180-3407 |
| 24/3418524CB1/ | 1-600, 106-138, 108-708, 121-527, 121-777, 121-780, 124-758, 160-398, 160-831, 195-665, 293-843, 297-843, 355-843, |
| 3270 | 366-843, 375-638, 519-1015, 714-1306, 788-1347, 804-972, 804-1008, 804-1164, 804-1219, 804-1232, 804-1266, |
| | 804-1344, 804-1346, 804-1438, 804-1452, 804-1534, 1043-1727, 1064-1227, 1080-1362, 1132-1653, 1144-1398, |
| | 1148-1632, 1164-1440, 1386-1641, 1387-1880, 1396-1564, 1465-2004, 1475-2114, 1511-2138, 1588-2108, |
| | 1921-3243, 3145-3270 |
| 25/7490407CB1/ | 1-534, 1-567, 229-570, 376-570 |
| 570 | |
| 26/700648CB1/ | 1-126, 1-249, 1-275, 1-332, 1-516, 8-332, 43-332, 111-332, 124-332, 129-375, 148-332, 157-332, 165-375, 174-332, |
| 7768 | 190-332, 197-332, 198-332, 249-332, 282-332, 285-332, 296-332, 369-471, 479-3440, 582-1185, 582-1227, 747-849, |
| | 1079-1504, 1129-1804, 1138-1472, 1138-4742, 1177-1701, 1271-1941, 1451-1767, 1453-7742, 1499-1724, |
| | 1536-1897, 1602-2097, 1660-1924, 1730-2218, 1745-2125, 1770-2384, 1791-2218, 1826-2457, 1880-2706, 2022-2575, |
| | 2022-2674, 2078-2717, 2185-3059, 2335-2859, 2406-2703, 2457-3098, 2475-2999, 2484-3136, 2533-3261, |
| | 2556-3100, 2642-3235, 2695-2870, 2695-3050, 2695-3124, 2695-3143, 2695-3167, 2695-3211, 2695-3226, 2695-3270, |
| | 2695-3284, 2695-3312, 2786-3054, 2850-3229, 2918-3531, 2949-3470, 2958-3220, 2980-3619, 3035-3494, |
| | 3090-3597, 3104-3619, 3104-3621, 3171-3696, 3186-3441, 3193-3619, 3193-3758, 3193-3807, 3215-3687, 3219-3831, |
| | 3229-3619, 3231-3957, 3245-3771, 3300-3901, 3399-3970, 3440-3751, 3461-3978, 3469-4068, 3527-4083, |
| | 3607-4145, 3610-3794, 3688-4226, 3734-3867, 3770-4145, 3878-4446, 3910-4069, 3972-4625, 4038-4576, |
| | 4040-4723, 4073-4618, 4089-4730, 4133-4516, 4183-4609, 4206-5056, 4250-4500, 4307-4595, 4310-4859, 4334-4948, |
| | 4336-5020, 4532-4982, 4624-5260, 4926-5254, 4951-5378, 4974-5527, 5132-5602, 5208-5712, 5208-5749, |
| | 5215-5501, 5253-5584, 5260-5814, 5295-5639, 5295-5775, 5363-5832, 5376-5651, 5381-5584, 5400-5865, 5409-5961, |
| | 5417-5584, 5426-5903, 5442-6047, 5442-6053, 5449-6140, 5450-5617, 5450-6035, 5450-6052, 5450-6168, |
| | 5501-6045, 5534-5826, 5534-5844, 5537-5812, 5548-6214, 5566-6197, 5582-5990, 5604-6274, 5612-5855, 5612-5879, |
| | 5612-5880, 5612-6160, 5614-6206, 5630-6297, 5710-6331, 5754-6214, 5797-6167, 5797-6254, 5802-6316, |
| | 5805-6184, 5811-6356, 5868-6341, 5890-6485, 5919-6603, 6010-6390, 6045-6581, 6109-6648, 6206-6801, 6207-6906, |
| | 6208-6619, 6214-6477, 6229-6826, 6260-6810, 6262-6514, 6264-6840, 6268-6885, 6294-6542, 6296-6772, |
| | 6301-6666, 6320-6618, 6321-6928, 6356-6828, 6369-6917, 6374-6936, 6376-6985, 6402-6941, 6402-6973, 6408-6830, |
| | 6419-6854, 6463-7127, 6472-7075, 6480-7113, 6507-6714, 6513-7052, 6516-6991, 6526-6767, 6526-6768, |
| | 6553-7127, 6554-7159, 6561-7241, 6583-7081, 6593-7084, 6593-7249, 6607-7245, 6608-7203, |
| | 6610-7064, 6639-7118, 6639-7172, 6661-7194, 6671-6939, 6673-6990, 6673-7143, 6673-7257, 6683-7532, 6688-7220, |
| | 6714-7532, 6730-6988, 6730-7025, 6730-7360, 6733-7532, 6736-6902, 6736-7004, 6736-7008, 6742-7399, |
| | 6745-7005, 6757-7196, 6758-7399, 6771-7134, 6779-7532, 6781-7302, 6789-7080, 6792-7532, 6799-7305, 6799-7339, |
| | 6809-7113, 6809-7508, 6816-7302, 6816-7311, 6827-7296, 6832-7491, 6835-7278, 6838-7081, 6847-7110, |
| | 6852-7699, 6853-7082, 6853-7436, 6855-7022, 6856-7532, 6865-7622, 6869-7108, 6877-7444, 6883-7201, 6884-7079, |
| | 6884-7149, 6893-7391, 6900-7130, 6927-7149, 6927-7178, 6927-7598, 6931-7095, 6967-7580, 6976-7513, |
| | 6979-7222, 6979-7233, 6990-7529, 6995-7571, 7021-7723, 7026-7438, 7065-7300, 7065-7653, 7068-7598, 7089-7739, |
| | 7094-7735, 7096-7377, 7099-7393, 7103-7746, 7104-7349, 7105-7735, 7106-7294, 7108-7740, 7113-7746, |
| | 7130-7735, 7132-7673, 7139-7711, 7150-7752, 7156-7410, 7160-7763, 7175-7609, 7175-7763, 7200-7453, 7205-7768, |
| | 7207-7460, 7217-7484, 7223-7742, 7243-7517, 7248-7746, 7251-7510, 7284-7562, 7289-7743, 7292-7746, |
| | 7293-7472, 7301-7743, 7306-7745, 7307-7746, 7310-7749, 7377-7438, 7596-7753 |
| 27/2744459CB1/ | 1-536, 1-580, 17-285, 17-578, 19-424, 45-602, 55-290, 56-345, 57-711, 111-704, 189-610, 231-733, 306-544, 383-546, |
| 2076 | 395-509, 517-1207, 530-1191, 545-999, 568-1069, 580-2011, 605-881, 606-881, 619-770, 619-791, 619-794, |
| | 619-1031, 619-1084, 645-1100, 645-1245, 680-1019, 681-834, 853-1276, 875-1155, 905-1127, 944-1211, 949-1404, |
| | 977-1556, 986-1161, 995-1368, 1073-1191, 1099-1342, 1119-1269, 1135-1405, 1141-1191, 1157-1420, 1157-1620, |
| | 1162-1474, 1162-1486, 1174-1369, 1188-1799, 1191-1609, 1208-1288, 1208-1485, 1269-1816, 1287-1545, 1314-1740, |
| | 1322-1577, 1361-1710, 1395-1654, 1421-1556, 1429-2051, 1432-2023, 1483-1759, 1484-1769, 1593-1869, |
| | 1593-2051, 1595-1926, 1601-1764, 1610-1861, 1619-1885, 1724-1948, 1772-2051, 1820-1962, 1934-2076 |
| 28/60204026CB1/ | 1-565, 35-322, 35-582, 35-768, 41-783, 46-646, 68-211, 138-578, 230-1092, 254-673, 463-1080, 648-1158, 722-926, |
| 2818 | 722-1037, 820-1152, 820-1197, 820-1274, 820-1279, 820-1369, 822-1139, 822-1376, 836-1072, 863-1101, 868-1335, |
| | 930-1190, 932-1445, 945-1196, 1015-1573, 1104-1379, 1104-1430, 1119-1406, 1119-1440, 1119-1710, 1146-1365, |
| | 1146-1597, 1177-1640, 1179-1606, 1179-1685, 1180-1479, 1181-1725, 1221-1435, 1221-1461, 1275-1580, |
| | 1284-1515, 1284-1862, 1293-1736, 1308-1654, 1314-1514, 1322-1564, 1353-1638, 1360-1858, 1364-1691, 1374-1615, |
| | 1398-2074, 1400-1555, 1400-1588, 1411-1708, 1420-1666, 1432-1683, 1437-1844, 1480-1673, 1480-1690, |
| | 1480-1767, 1492-2152, 1504-2141, 1508-1743, 1518-1804, 1519-1814, 1549-1810, 1559-1970, 1561-1817, 1563-1821, |
| | 1565-2000, 1573-1703, 1591-1835, 1618-1935, 1631-2078, 1636-1826, 1640-1871, 1649-1909, 1666-1943, |
| | 1671-1905, 1671-2053, 1688-1947, 1694-2119, 1704-2275, 1706-1972, 1706-2227, 1710-2259, 1710-2452, 1742-2000, |
| | 1754-2451, 1755-2404, 1772-2017, 1780-2136, 1787-2092, 1790-2089, 1798-2555, 1800-2042, |
| | 1839-2110, 1849-2079, 1865-2034, 1865-2452, 1895-2400, 1895-2456, 1895-2458, 1896-2153, 1909-2177, 1914-2423, |
| | 1930-2243, 1931-2743, 1936-2279, 1959-2445, 1980-2208, 1985-2288, 2016-2633, 2020-2299, 2020-2301, |
| | 2031-2727, 2050-2278, 2083-2343, 2083-2654, 2083-2747, 2097-2400, 2107-2367, 2115-2542, 2120-2798, 2122-2425, |
| | 2122-2754, 2128-2762, 2142-2704, 2142-2705, 2149-2389, 2149-2804, 2154-2768, 2159-2447, 2163-2796, |
| | 2164-2783, 2165-2408, 2168-2708, 2171-2479, 2174-2818, 2175-2431, 2182-2440, 2199-2796, 2200-2787, 2213-2489, |
| | 2218-2476, 2228-2790, 2243-2757, 2243-2818, 2245-2488, 2262-2818, 2268-2795, 2271-2818, 2272-2573, |
| | 2273-2345, 2274-2465, 2290-2792, 2300-2803, 2302-2818, 2318-2455, 2318-2764, 2319-2792, 2320-2761, 2320-2789, |
| | 2320-2818, 2331-2604, 2332-2806, 2334-2809, 2335-2806, 2335-2818, 2339-2804, 2340-2815, 2342-2809, |
| | 2345-2740, 2352-2809, 2353-2649, 2353-2804, 2355-2634, 2356-2412, 2358-2802, 2361-2790, 2362-2767, 2373-2623, |
| | 2378-2643, 2378-2818, 2380-2807, 2381-2805, 2382-2807, 2386-2806, 2387-2809, 2390-2815, 2393-2689, |
| | 2393-2717, 2404-2792, 2410-2806, 2413-2806, 2417-2806, 2418-2790, 2422-2799, 2423-2807, 2425-2808, |
| | 2430-2806, 2432-2754, 2432-2806, 2436-2806, 2440-2802, 2445-2791, 2457-2760, 2457-2811, 2460-2625, 2463-2810, |
| | 2467-2808, 2475-2811, 2476-2806, 2484-2806, 2502-2809, 2509-2804, 2513-2809, 2539-2716, 2540-2818, |
| | 2552-2818, 2554-2812, 2558-2818, 2560-2818, 2565-2806, 2581-2806, 2583-2815, 2605-2806, 2628-2804, 2633-2766, |
| | 2633-2818, 2648-2818, 2665-2818 |
| 29/7473835CB1/ | 1-2057, 93-2057, 133-2057 |
| 2057 | |
| 30/8186336CB1/ | 1-163, 1-217, 1-624, 164-491, 249-295, 296-632 |
| 632 | |
| 31/7493330CB1/ | 1-202, 1-517, 4-715, 8-580, 36-391, 229-733, 254-812, 254-977, 257-986, 258-805, 287-389, 296-359, 386-1243, |
| 3044 | 390-899, 405-940, 461-600, 624-945, 642-944, 644-1351, 655-1208, 671-1351, 682-1343, 719-1314, 771-1374, 841-1439, |
| | 850-1542, 903-1583, 911-1524, 950-1219, 955-1578, 969-1539, 981-1717, 1024-1366, 1031-1317, 1038-1497, |
| | 1038-1539, 1085-1344, 1106-1885, 1144-1846, 1160-1328, 1186-1425, 1189-1636, 1198-1709, 1207-1833, |
| | 1225-1473, 1225-1575, 1235-1326, 1293-1914, 1331-1936, 1341-1881, 1347-1945, 1376-2022, 1399-2028, 1399-2031, |
| | 1400-2269, 1422-2178, 1440-2071, 1464-1925, 1474-2269, 1560-2269, 1565-2184, 1614-1864, 1631-1895, |
| | 1631-2226, 1633-2179, 1639-2333, 1651-2281, 1661-2292, 1669-2243, 1687-2299, 1706-2299, 1746-2147, 1759-2326, |
| | 1764-2402, 1783-1939, 1784-2299, 1825-2418, 1835-2488, 1858-2526, 1909-2441, 1920-2397, 1920-2430, |
| | 1920-2441, 1920-2442, 1945-2294, 1946-2346, 1949-2441, 1953-2443, 1978-2291, 1981-2358, 2001-2369, 2006-2257, |
| | 2022-2447, 2051-2315, 2058-2353, 2067-2304, 2067-2308, 2067-2522, 2067-2549, 2067-2563, 2067-2638, |
| | 2067-2712, 2067-2815, 2067-2950, 2080-2342, 2080-2353, 2105-2390, 2109-2368, 2120-2408, 2139-2365, |
| | 2147-2815, 2176-2379, 2176-2811, 2185-2447, 2211-2360, 2232-2782, 2238-2444, 2243-2288, 2260-2551, 2264-2460, |
| | 2264-2743, 2269-2886, 2270-3044, 2275-2472, 2275-2477, 2285-2414, 2286-2551, 2308-2892, 2333-2680, |
| | 2352-2853 |
| 32/7487969CB1/ | 1-693, 73-545 |
| 693 | |
| 33/2655990CB1/ | 1-593, 1-3223, 6-347, 10-733, 12-238, 32-735, 33-412, 40-341, 44-391, 45-292, 55-350, 55-472, 86-538, 156-562, |
| 3323 | 168-414, 168-648, 289-508, 470-1096, 477-798, 534-1100, 576-1298, 784-983, 786-941, 836-1100, 858-1118, 875-1466, |
| | 971-1509, 1044-1646, 1060-1330, 1074-1412, 1074-1573, 1107-1539, 1109-1721, 1109-1725, 1109-1760, |
| | 1134-1432, 1303-1539, 1334-1917, 1402-1695, 1450-1726, 1468-1722, 1537-1816, 1548-2149, 1622-1886, 1690-1911, |
| | 1729-1996, 1729-2098, 1729-2206, 1729-2215, 1729-2240, 1729-2243, 1729-2245, 1729-2285, 1729-2301, |
| | 1729-2309, 1729-2386, 1729-2391, 1729-2414, 1777-2472, 1800-2502, 1857-2374, 1863-2097, 1865-2100, 1903-2125, |
| | 1967-2225, 1999-2296, 2001-2455, 2058-2488, 2106-2498, 2133-2610, 2205-2437, 2206-3198, 2228-3179, |
| | 2229-3209, 2258-3176, 2300-3185, 2399-2673, 2405-2622, 2507-2769, 2563-2840, 2572-3198, 2616-2819, 2616-2843, |
| | 2616-3129, 2616-3213, 2633-2875, 2657-2904, 2882-3263, 3051-3323 |
| 34/71768694CB1/ | 1-596, 196-806, 219-481, 306-567, 320-747, 499-1178, 514-1223, 623-1267, 643-1256, 709-1054, 737-1012, 737-1023, |
| 2959 | 762-1389, 767-1457, 784-1419, 815-1067, 821-1459, 841-1280, 851-1511, 858-1062, 882-1423, 905-1486, |
| | 967-1456, 1009-1286, 1032-1555, 1061-1238, 1068-1662, 1076-1590, 1082-1250, 1082-1365, 1133-1587, 1169-1742, |
| | 1235-1507, 1235-1743, 1267-1518, 1358-1930, 1421-1958, 1437-1647, 1454-1663, 1463-2096, 1651-1895, |
| | 1692-1937, 1692-2071, 1694-2337, 1699-1837, 1710-1875, 1824-2066, 1824-2333, 1838-2125, 1918-2222, 1926-2141, |
| | 1926-2510, 1951-2176, 1964-2156, 1970-2254, 1987-2226, 1991-2489, 2121-2773, 2122-2392, 2143-2428, |
| | 2143-2429, 2151-2357, 2270-2531, 2281-2566, 2286-2930, 2310-2931, 2329-2618, 2337-2590, 2354-2482, 2359-2501, |
| | 2438-2676, 2438-2780, 2438-2790, 2453-2643, 2453-2773, 2453-2790, 2687-2938, 2688-2918, 2688-2953, |
| | 2713-2959, 2750-2959, 2780-2959, 2796-2923 |
| 35/5079019CB1/ | 1-420, 51-420, 286-319, 332-475, 332-693, 338-678, 358-441, 358-558, 361-515, 366-558, 433-685, 499-538, 505-538 |
| 693 | |
| 36/894500CB1/ | 1-707, 80-4876, 225-722, 495-1159, 570-3029, 793-1040, 793-1325, 860-1520, 889-1727, 905-1480, 1232-1748, |
| 4938 | 1542-1822, 1546-2158, 1599-1808, 1599-1892, 1599-1930, 1599-1960, 1599-2023, 1599-2025, 1599-2027, 1599-2069, |
| | 1599-2195, 1686-2196, 1687-2070, 1688-1972, 1691-2255, 1691-2275, 1788-1975, 1826-2022, 1864-1885, |
| | 1882-2125, 1882-2139, 1972-2245, 1981-2614, 1996-2336, 2076-2608, 2089-2115, 2109-2148, 2128-2284, 2155-2663, |
| | 2176-2647, 2176-2857, 2193-2825, 2219-2748, 2242-2921, 2251-2895, 2361-2645, 2424-2983, 2504-2964, |
| | 2566-2898, 2588-3228, 2648-2903, 2943-3615, 2943-3647, 2943-3686, 2943-3697, 2943-3711, 2943-3715, 2944-3190, |
| | 2944-3522, 2944-3596, 2996-3839, 3027-3818, 3054-3820, 3090-3900, 3107-3689, 3116-3686, 3153-3966, |
| | 3277-4054, 3360-4126, 3388-4020, 3425-3838, 3439-4165, 3456-4301, 3505-3782, 3599-4458, 3652-4269, 3703-4247, |
| | 3862-4682, 3887-4688, 4005-4599, 4119-4853, 4123-4909, 4194-4915, 4218-4938, 4237-4915, 4252-4929 |
| 37/7497866CB1/ | 1-800, 1-1732, 17-796, 32-795, 53-544, 57-601, 153-389, 153-773, 157-758, 465-841, 693-946, 803-1007, 820-1355, |
| 2244 | 863-1141, 863-1271, 1267-1522, 1274-1533, 1297-1732, 1454-1715, 1461-2053, 1524-1791, 1524-2057, 1573-1842, |
| | 1586-1827, 1649-1910, 1780-2038, 1780-2241, 1786-2244, 1833-2034, 1886-2146 |
| 38/832718CB1/ | 1-394, 125-349, 208-391, 225-698, 228-623, 232-391, 246-394, 626-6187, 844-1177, 1150-1715, 1223-1743, 1377-1879, |
| 9353 | 1384-1671, 1418-1588, 3199-3844, 3199-3855, 3199-3856, 3243-3768, 3294-4031, 3520-4225, 3546-4124, |
| | 3546-4142, 3546-4146, 3546-4188, 3546-4210, 3546-4221, 3546-4247, 3546-4281, 3549-3992, 3549-4264, 3555-4326, |
| | 3715-4207, 3718-4209, 3719-4349, 3719-4352, 3725-4340, 3760-4416, 3926-4340, 4340-4556, 5776-6307, |
| | 5776-6498, 5784-6403, 5805-6306, 5977-6651, 5978-6631, 6000-6707, 6012-6653, 6018-6707, 6059-6645, 6068-6535, |
| | 6070-6657, 6078-6616, 6096-6911, 6171-6720, 6185-6696, 6185-6827, 6205-6895, 6225-6757, 6229-6734, |
| | 6262-6895, 6274-6745, 6358-6949, 6358-6957, 6379-6886, 6393-6843, 6459-7033, 6543-7204, 6555-7075, 6563-7136, |
| | 6580-7132, 6601-7098, 6611-7185, 6611-7187, 6633-7216, 6690-7230, 6716-7255, 6716-7298, 6716-7385, |
| | 6718-7365, 6730-7284, 6734-7315, 6792-7307, 6842-7484, 6934-7579, 6939-7467, 6950-7555, 6993-7433, 7020-7556, |
| | 7132-7616, 7257-7543, 7271-7835, 7308-7878, 7362-8045, 7384-7877, 7394-8063, 7414-8091, 7424-7907, |
| | 7445-8088, 7468-8099, 7485-8064, 7512-8095, 7517-8094, 7596-8140, 7609-8161, 7664-8180, 7673-8308, 7683-8373, |
| | 7692-8113, 7744-8210, 7773-8317, 7784-8469, 7802-8375, 7815-8092, 7819-8450, 7821-8499, 7823-8529, |
| | 7828-8160, 7830-8588, 7854-8358, 7858-8420, 7865-8444, 7922-8548, 7923-8570, 7942-8184, 7945-8535, 7968-8249, |
| | 7969-8547, 8014-8673, 8020-8525, 8044-8559, 8049-8689, 8063-8375, 8063-8540, 8077-8590, 8082-8591, |
| | 8084-8325, 8095-8804, 8110-8650, 8117-8760, 8119-8646, 8143-8466, 8144-8781, 8149-8699, 8152-8698, 8164-8813, |
| | 8188-8843, 8189-8774, 8189-8838, 8199-8376, 8199-8877, 8214-8693, 8237-8709, 8237-8865, 8246-8790, |
| | 8292-8516, 8314-8913, 8314-9096, 8321-8779, 8335-8470, 8347-8728, 8411-8992, 8423-9031, 8534-8652, 8543-9256, |
| | 8574-9261, 8576-9190, 8591-8926, 8600-9141, 8603-9093, 8610-8766, 8637-8916, 8706-9316, 8709-9353, |
| | 8714-9268, 8723-8970, 8723-9157, 8723-9266, 8772-9221, 8776-9315, 8781-9353 |
| 39/7497717CB1/ | 1-394, 125-349, 208-391, 225-698, 228-623, 232-391, 246-394, 626-4936, 844-1177, 1150-1715, 1223-1743, 1377-1879, |
| 9449 | 1384-1671, 1418-1588, 3199-3844, 3199-3855, 3199-3856, 3243-3768, 3294-4031, 3520-4225, 3546-4124, |
| | 3546-4142, 3546-4146, 3546-4188, 3546-4210, 3546-4221, 3546-4247, 3546-4269, 3546-4281, 3549-3992, 3549-4264, |
| | 3555-4326, 3715-4207, 3718-4209, 3719-4343, 3719-4352, 3725-4340, 3760-4414, 3926-4340, 4057-4777, |
| | 4143-4716, 4146-4802, 4155-4785, 4179-4751, 4262-4866, 4340-4556, 4383-5046, 4621-5065, 5002-5061, 5033-6283, |
| | 5151-5400, 5220-5846, 5244-5604, 5326-5584, 5326-5753, 5493-6095, 5872-6403, 5872-6594, 5880-6499, |
| | 5901-6402, 6073-6747, 6074-6727, 6096-6803, 6108-6749, 6114-6803, 6155-6741, 6164-6631, 6166-6753, 6174-6712, |
| | 6192-7007, 6267-6816, 6281-6792, 6281-6923, 6301-6991, 6321-6853, 6325-6830, 6358-6991, 6370-6841, |
| | 6454-7045, 6454-7053, 6475-6982, 6489-6939, 6555-7129, 6639-7300, 6651-7171, 6659-7232, 6676-7228, 6697-7194, |
| | 6707-7281, 6707-7283, 6729-7312, 6786-7326, 6812-7351, 6812-7394, 6812-7481, 6814-7461, 6826-7380, |
| | 6830-7411, 6888-7403, 6938-7580, 7030-7675, 7035-7563, 7046-7651, 7089-7529, 7116-7652, 7228-7712, 7353-7639, |
| | 7367-7931, 7404-7974, 7458-8141, 7480-7973, 7490-8159, 7510-8187, 7520-8003, 7541-8184, 7564-8195, |
| | 7581-8160, 7608-8191, 7613-8190, 7692-8236, 7705-8257, 7760-8276, 7769-8404, 7779-8469, 7788-8209, 7840-8306, |
| | 7869-8413, 7880-8565, 7898-8471, 7911-8188, 7915-8546, 7917-8595, 7919-8625, 7924-8256, 7926-8684, |
| | 7950-8454, 7954-8516, 7961-8540, 8018-8644, 8019-8666, 8038-8280, 8041-8631, 8064-8345, 8065-8643, 8110-8769, |
| | 8116-8621, 8140-8655, 8145-8785, 8159-8471, 8159-8636, 8173-8686, 8178-8687, 8180-8421, 8191-8900, |
| | 8206-8746, 8213-8856, 8215-8742, 8239-8562, 8240-8877, 8245-8795, 8248-8794, 8260-8909, 8284-8939, 8285-8870, |
| | 8285-8934, 8295-8472, 8295-8973, 8310-8789, 8331-8479, 8333-8805, 8333-8961, 8342-8886, 8388-8612, |
| | 8410-9009, 8410-9192, 8417-8875, 8431-8566, 8443-8824, 8507-9088, 8519-9127, 8639-9352, 8670-9357, 8672-9286, |
| | 8687-9022, 8696-9237, 8699-9189, 8706-8862, 8733-9012, 8802-9412, 8805-9449, 8810-9364, 8819-9066, |
| | 8819-9253, 8819-9362, 8868-9317, 8872-9411, 8877-9449 |
| 40/7506420CB1/ | 1-635, 1-2065, 2-765, 286-662, 514-767, 624-828, 641-1176, 684-962, 684-1092, 769-1442, 1088-1343, 1095-1354, |
| 2065 | 1104-1191, 1118-1553, 1275-1536, 1282-1874, 1345-1612, 1345-1878, 1394-1500, 1394-1663, 1395-1989, 1407-1629, |
| | 1425-1827, 1470-1731, 1601-1859, 1601-2062, 1607-2065, 1618-1738, 1654-1855, 1707-1967, 1768-2019 |

TABLE 5

| Polynucleotide | | |
|---|---|---|
| SEQ ID NO: | Incyte Project ID: | Representative Library |
| 22 | 1966295CB1 | PROSNOT15 |
| 23 | 113399CB1 | PENITUT01 |
| 24 | 3418524CB1 | PITUNON01 |
| 26 | 700648CB1 | KIDNTUE01 |
| 27 | 2744459CB1 | BRAUNOT01 |
| 28 | 60204026CB1 | PROSNOT14 |
| 30 | 8186336CB1 | EYERNON01 |
| 31 | 7493330CB1 | BRAIFEC01 |
| 33 | 2655990CB1 | BRABDIK02 |
| 34 | 71768694CB1 | BRSTNOT04 |
| 35 | 5079019CB1 | BRONNOT02 |
| 36 | 894500CB1 | BRANDIN01 |
| 37 | 7497866CB1 | COLNNOT16 |
| 38 | 832718CB1 | BRAIFER05 |
| 39 | 7497717CB1 | BRAIFER05 |
| 40 | 7506420CB1 | COLNNOT16 |

TABLE 6

| Library | Vector | Library Description |
|---|---|---|
| BRABDIK02 | PSPORT1 | This amplified and normalized library was constructed using pooled cDNA from three different donors. cDNA was |
| | | generated using mRNA isolated from diseased vermis tissue removed from a 79-year-old Caucasian female (donor A) |
| | | who died from pneumonia, an 83-year-old Caucasian male (donor B) who died from congestive heart failure, and an |
| | | 87-year-old Caucasian female (donor C) who died from esophageal cancer. Pathology indicated severe Alzheimer's |
| | | disease in donors A & B and moderate Alzheimer's disease in donor C. Patient history included glaucoma, pseudophakia, |
| | | gastritis with gastrointestinal bleeding, peripheral vascular disease, chronic obstructive pulmonary disease, seizures, |
| | | tobacco abuse in remission, and transitory ischemic attacks in donor A; Parkinson's disease and atherosclerosis in donor |
| | | B; hypertension, coronary artery disease, cerebral vascular accident, and hypothyroidism in donor C. Family history |
| | | included Alzheimer's disease in the mother and sibling(s) of donor A. Independent clones from this amplified library |
| | | were normalized in one round using conditions adapted Soares et al., PNAS (1994) 91: 9228-9232 and Bonaldo |
| | | et al., Genome Research 6 (1996): 791, except that a significantly longer (48 hours/round) reannealing hybridization was |
| | | used. |
| BRAIFEC01 | pINCY | This large size-fractionated library was constructed using RNA isolated from brain tissue removed from a Caucasian male |
| | | fetus who was stillborn with a hypoplastic left heart at 23 weeks' gestation. |
| BRAIFER05 | pINCY | Library was constructed using RNA isolated from brain tissue removed from a Caucasian male fetus who was stillborn |
| | | with a hypoplastic left heart at 23 weeks' gestation. |
| BRANDIN01 | pINCY | This normalized pineal gland tissue library was constructed from .4 million independent clones from a pineal gland tissue |
| | | library from two different donors. Starting RNA was made from pooled pineal gland tissue removed from two Caucasian |
| | | females: a 68-year-old (donor A) who died from congestive heart failure and a 79-year-old (donor B) who died from |
| | | pneumonia. Neuropathology for donor A indicated mild to moderate Alzheimer disease, atherosclerosis, and multiple |
| | | infarctions. Neuropathology for donor B indicated severe Alzheimer disease, arteriolosclerosis, cerebral amyloid |
| | | angiopathy and multiple infarctions. There were diffuse and neuritic amyloid plaques and neurofibrillary tangles |
| | | throughout the brain sections examined in both donors. Patient history included diabetes mellitus, rheumatoid arthritis, |
| | | hyperthyroidism, amyloid heart disease, and dementia in donor A; and pseudophakia, gastritis with bleeding, glaucoma, |
| | | peripheral vascular disease, COPD, delayed onset tonic/clonic seizures, and transient ischemic attack in donor B. The |
| | | library was normalized in one round using conditions adapted from Soares et al., PNAS (1994) 91: 9228-9232 and |
| | | Bonaldo et al., Genome Research 6 (1996): 791, except that a significantly longer (48 hours/round) reannealing |
| | | hybridization was used. |
| BRAUNOT01 | pINCY | The library was constructed using RNA isolated from caudate/putamen/nucleus accumbens tissue removed from the brain |
| | | of a 35-year-old Caucasian male who died from cardiac failure. Pathology indicated moderate leptomeningeal fibrosis and |
| | | multiple microinfarctions of the cerebral neocortex. Microscopically, the cerebral hemisphere revealed moderate fibrosis |
| | | of the leptomeninges with focal calcifications. There was evidence of shrunken and slightly eosinophilic pyramidal |
| | | neurons throughout the cerebral hemispheres. In addition, scattered throughout the cerebral cortex, there were multiple |
| | | small microscopic areas of cavitation with surrounding gliosis. Patient history included dilated cardiomyopathy, |
| | | congestive heart failure, cardiomegaly and an enlarged spleen and liver. |
| BRONNOT02 | pINCY | Library was constructed using RNA isolated from right lower lobe bronchial tissue removed from a pool of 9 |
| | | nonasthmatic Caucasian male and female donors, 18-to 55-years-old during bronchial pinch biopsies. Patient history |
| | | included atopy as determined by positive skin tests to common aero-allergens with no bronchial hyperresponsiveness to |
| | | histamine. The donors were not current smokers and had no history of alcohol or drug abuse. |
| BRSTNOT04 | PSPORT1 | Library was constructed using RNA isolated from breast tissue removed from a 62-year-old East Indian female during a |
| | | unilateral extended simple mastectomy. Pathology for the associated tumor tissue indicated an invasive grade 3 ductal |
| | | carcinoma. Patient history included benign hypertension, hyperlipidemia, and hematuria. Family history included |
| | | cerebrovascular and cardiovascular disease, hyperlipidemia, and liver cancer. |
| COLNNOT16 | pINCY | Library was constructed using RNA isolated from sigmoid colon tissue removed from a 62-year-old Caucasian male |
| | | during a sigmoidectomy and permanent colostomy. |
| EYERNON01 | PSPORT1 | This normalized pooled retina tissue library was constructed from independent clones from a pooled retina tissue library. |
| | | Starting RNA was made from pooled retina tissue removed from 34 male and female donors, aged 9 to 80-years-old. The |
| | | library was normalized in one round using conditions adapted from Soares et al., PNAS (1994) 91: 9228-9232 and |
| | | Bonaldo et al., Genome Research 6 (1996): 791, except that a significantly longer (48 hours/round) reannealing |
| | | hybridization was used. |
| KIDNTUE01 | PCDNA2.1 | This 5′ biased random primed library was constructed using RNA isolated from kidney tumor tissue removed from a 46- |
| | | year-old Caucasian male during nephroureterectomy. Pathology indicated grade 2 renal cell carcinoma, clear-cell type, |
| | | forming a mass in the upper pole. The patient presented with kidney cancer, backache, headache, malignant hypertension, |
| | | nausea, and vomiting. Previous surgeries included repair of indirect inguinal hernia. Patient medications included Lasix, |
| | | Inderal, and Procardia. Family history included cerebrovascular accident in the mother; acute myocardial infarction and |
| | | atherosclerotic coronary artery disease in the father; and type II diabetes in the sibling(s). |
| PENITUT01 | pINCY | Library was constructed using RNA isolated from tumor tissue removed from the penis of a 64-year-old Caucasian male |
| | | during penile amputation. Pathology indicated a fungating invasive grade 4 squamous cell carcinoma involving the inner |
| | | wall of the foreskin and extending onto the glans penis. Patient history included benign neoplasm of the large bowel, |
| | | atherosclerotic coronary artery disease, angina pectoris, gout, and obesity. Family history included malignant pharyngeal |
| | | neoplasm, chronic lymphocytic leukemia, and chronic liver disease. |
| PITUNON01 | pINCY | This normalized pituitary gland tissue library was constructed from 6.92 million independent clones from a pituitary |
| | | gland tissue library. Starting RNA was made from pituitary gland tissue removed from a 55-year-old male who died from |
| | | chronic obstructive pulmonary disease. Neuropathology indicated there were no gross abnormalities, other than mild |
| | | ventricular enlargement. There was no apparent microscopic abnormality in any of the neocortical areas examined, except |
| | | for a number of silver positive neurons with apical dendrite staining, particularly in the frontal lobe. The significance |
| | | of this was undetermined. The only other microscopic abnormality was that there was prominent silver staining with |
| | | some swollen axons in the CA3 region of the anterior and posterior hippocampus. Microscopic sections of the cerebellum |
| | | revealed mild Bergmann's gliosis in the Purkinje cell layer. Patient history included schizophrenia. The library was |
| | | normalized in two rounds using conditions adapted from Soares et al., PNAS (1994) 91: 9228-9232 and Bonaldo et al., |
| | | Genome Research (1996) 6: 791, except that a significantly longer (48 hours/round) reannealing hybridization was used. |
| PROSNOT14 | pINCY | Library was constructed using RNA isolated from diseased prostate tissue removed from a 60-year-old Caucasian male |
| | | during radical prostatectomy and regional lymph node excision. Pathology indicated adenofibromatous hyperplasia. |
| | | Pathology for the associated tumor tissue indicated an adenocarcinoma (Gleason grade 3 + 4). The patient presented with |
| | | elevated prostate specific antigen (PSA). Patient history included a kidney cyst and hematuria. Family history included |
| | | benign hypertension, cerebrovascular disease, and arteriosclerotic coronary artery disease. |
| PROSNOT15 | pINCY | Library was constructed using RNA isolated from diseased prostate tissue removed from a 66-year-old Caucasian male |
| | | during radical prostatectomy and regional lymph node excision. Pathology indicated adenofibromatous hyperplasia. |
| | | Pathology for the associated tumor tissue indicated an adenocarcinoma (Gleason grade 2 + 3). The patient presented with |
| | | elevated prostate specific antigen (PSA). Family history included prostate cancer, secondary bone cancer, and benign |
| | | hypertension. |

TABLE 7

| Program | Description | Reference | Parameter Threshold |
|---|---|---|---|
| HMMER | An algorithm for searching a query sequence against | Krogh, A. et al. (1994) J. Mol. Biol. | PFAM, INCY, SMART or |
| | hidden Markov model (HMM)-based databases of | 235: 1501-1531; Sonnhammer, E. L. L. et al. | TIGRFAM hits: Probability |
| | protein family consensus sequences, such as PFAM, | (1988) Nucleic Acids Res. 26: 320-322; | value = 1.0E−3 or less; Signal |
| | INCY, SMART and TIGRFAM. | Durbin, R. et al. (1998) Our World View, in | peptide hits: Score = 0 or |
| | | a Nutshell, Cambridge Univ. Press, pp. 1-350. | greater |
| ProfileScan | An algorithm that searches for structural and | Gribskov, M. et al. (1988) CABIOS 4: 61-66; | Normalized quality score ≧ |
| | sequence motifs in protein sequences that match | Gribskov, M. et al. (1989) Methods | GCG specified “HIGH” value |
| | sequence patterns defined in Prosite. | Enzymol. 183: 146-159; Bairoch, A. et al. | for that particular Prosite |
| | | (1997) Nucleic Acids Res. 25: 217-221. | motif. Generally, score = |
| | | | 1.4-2.1. |
| Phred | A base-calling algorithm that examines automated | Ewing, B. et al. (1998) Genome Res. 8: 175-185; | |
| | sequencer traces with high sensitivity and probability. | Ewing, B. and P. Green (1998) Genome | |
| | | Res. 8: 186-194. | |
| Phrap | A Phils Revised Assembly Program including | Smith, T. F. and M. S. Waterman (1981) Adv. | Score = 120 or greater; Match |
| | SWAT and CrossMatch, programs based on efficient | Appl. Math. 2: 482-489; Smith, T. F. and | length = 56 or greater |
| | implementation of the Smith-Waterman algorithm, | M. S. Waterman (1981) J. Mol. Biol. 147: 195- | |
| | useful in searching sequence homology and | 197; and Green, P., University of | |
| | assembling DNA sequences. | Washington, Seattle, WA. | |
| Consed | A graphical tool for viewing and editing Phrap | Gordon, D. et al. (1998) Genome Res. 8: | |
| | assemblies. | 195-202. | |
| SPScan | A weight matrix analysis program that scans protein | Nielson, H. et al. (1997) Protein Engineering | score = 3.5 or greater |
| | sequences for the presence of secretory signal | 10: 1-6; Claverie, J. M. and S. Audic (1997) | |
| | peptides. | CABIOS 12: 431-439. | |
| TMAP | A program that uses weight matrices to delineate | Persson, B. and P. Argos (1994) J. Mol. Biol. | |
| | transmembrane segments on protein sequences and | 237: 182-192; Persson, B. and P. Argos | |
| | determine orientation. | (1996) Protein Sci. 5: 363-371. | |
| TMHMMER | A program that uses a hidden Markov model | Sonnhammer, E. L. et al. (1998) proc. Sixth | |
| | (HMM) to delineate transmembrane segments on | Intl. Conf. On Intelligent Systems for Mol. | |
| | protein sequences and determine orientation. | Biol., Glasgow et al., eds., The Am. Assoc. | |
| | | for Artificial Intelligence (AAAI) Press, | |
| | | Menlo Park, CA, and MIT Press, Cambridge, | |
| | | MA, pp. 175-182. | |
| Motifs | A program that searches amino acid sequences for | Bairoch, A. et al. (1997) Nucleic Acids Res. | |
| | patterns that matched those defined in Prosite. | 25: 217-221; Wisconsin Package Program | |
| | | Manual, version 9, page M51-59, Genetics | |
| | | Computer Group, Madison, WI. | |

TABLE 8

| SEQ | | | | | | | | | | Caucasian | African | Asian | Hispanic |
|---|---|---|---|---|---|---|---|---|---|---|---|---|---|
| ID | | | | EST | CB1 | EST | | | Amino | Allele 1 | Allele 1 | Allele 1 | Allele 1 |
| NO: | PID | EST ID | SNP ID | SNP | SNP | Allele | Allele 1 | Allele 2 | Acid | frequency | frequency | frequency | frequency |
| 40 | 7506420 | 8520949H1 | SNP00142707 | 595 | 319 | A | G | A | Y105 | n/a | n/a | n/a | n/a |

1. An isolated polypeptide selected from the group consisting of:
a) a polypeptide comprising an amino acid sequence selected from the group consisting of SEQ ID NO: 1-20,
b) a polypeptide comprising a naturally occurring amino acid sequence at least 90% identical to an amino acid sequence selected from the group consisting of SEQ ID NO: 1-3, SEQ ID NO:5-9, and SEQ ID NO: 13-17,
c) a polypeptide comprising a naturally occurring amino acid sequence at least 91% identical to an amino acid sequence selected from the group consisting of SEQ ID NO: 10 and SEQ ID NO: 12,
d) a polypeptide comprising a naturally occurring amino acid sequence at least 97% identical to an amino acid sequence selected from the group consisting of SEQ ID NO: 18-19,
e) a polypeptide comprising a naturally occurring amino acid sequence at least 98% identical to an amino acid sequence selected from the group consisting of SEQ ID NO:4 and SEQ ID NO: 11,
f) a polypeptide consisting essentially of a naturally occurring amino acid sequence at least 90% identical to the amino acid sequence of SEQ ID NO:20,
g) a biologically active fragment of a polypeptide having an amino acid sequence selected from the group consisting of SEQ ID NO: 1-20, and
h) an immunogenic fragment of a polypeptide having an amino acid sequence selected from the group consisting of SEQ ID NO: 1-20.

2. An isolated polypeptide of claim 1 comprising an amino acid sequence selected from the group consisting of SEQ ID NO: 1-20.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:21-40.

6. A recombinant polynucleotide comprising a promoter sequence operably linked to a polynucleotide of claim 3.

7. A cell transformed with a recombinant polynucleotide of claim 6.

8. A transgenic organism comprising a recombinant polynucleotide of claim 6.

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO: 1-20.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:21-40,
b) a polynucleotide comprising a naturally occurring polynucleotide sequence at least 90% identical to a polynucleotide sequence selected from the group consisting of SEQ ID NO:21-23, and SEQ ID NO:25-40,
c) a polynucleotide comprising a naturally occurring polynucleotide sequence at least 98% identical to the amino acid sequence of SEQ ID NO:24,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO: 1-20.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (canceled)

22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (canceled)

25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. A method of screening for a compound that modulates the activity of the polypeptide of claim 1, the method comprising:
a) combining the polypeptide of claim 1 with at least one test compound under conditions permissive for the activity of the polypeptide of claim 1,
b) assessing the activity of the polypeptide of claim 1 in the presence of the test compound, and
c) comparing the activity of the polypeptide of claim 1 in the presence of the test compound with the activity of the polypeptide of claim 1 in the absence of the test compound, wherein a change in the activity of the polypeptide of claim 1 in the presence of the test compound is indicative of a compound that modulates the activity of the polypeptide of claim 1.

28. (canceled)

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-95. (canceled)


SEQUENCE LISTING (40 sequences)

SEQ ID NO: 1 - protein, 144 aa, Homo sapiens
  misc_feature: Incyte ID No 7488243CD1
MQAIKRVVVGDRAAGKTCLLTGYTTNSFPGDYILTVFDNCSANVMVLGKSVYLGLWNTTGQETMPPILSAHRRALNLLFPWSPTSFETAGAKWYPKVWHHYFPNTFVILVGIELDVREPLYTLLRKWWHLHTQCQVFGTDLIFP

SEQ ID NO: 2 - protein, 665 aa, Homo sapiens
  misc_feature: Incyte ID No 1966295CD1
MSEQSCQMSELRLLLLGKCRSGKSATGNAILGKHVFKSKFSDQTVIKMCQRESWVLRERKVVVIDTPDLFSSIACAEDKQRNIQHCLELSAPSLHALLLVIAIGHFTREDEETAKGIQQVFGAEARRHIIIVFTRKDDLGDDLLQDFIEKNKPLKQLVQDYEGRYCIFNNKTNSKDEQITQVLELLRKVESLVNTNGGPYHVNFKTEGSRFQDCVNEAASQEGDKPQGPRERQLQSTGPEQNPGTSELTVLLVGKRGAGKSAAGNSILGRQAFQTGFSEQSVTQSFLSESRSWRKKKVSIIDAPDISSLKNIDSEVRKHICTGPHAFLLVTPLGFYTKNDEAVLSTIQNNFGEKFFEYMIILLTRKEDLGDQDLDTFLRNSNKALYGLIQKCKNRYSAFNYRATGEEEQRQADELLEKIESMVHQNGNKHCVFREKETLNIVLVGRSGTGKSATGNSILGSLVFTSRLRAQPVTKTSQSGRRTWDGQEVVVVDTPSFNQMLDVEKDPSRLEEEVKRCLSCCEKGDTFFVLVFQLGRFTEEDKTAVAKLEAIFGADFTKYAIMLFTRKEDLGAGNLEDFMKNSDNKALRRIFKKCGRRVCAFNNKETGQAQETQVKALLTKVNDLRKESGWSGYPHTQENVSKLIKNVQEMSQAEKLLKNLIGILQ

SEQ ID NO: 3 - protein, 574 aa, Homo sapiens
  misc_feature: Incyte ID No 113399CD1
MALKGRALYDFHSENKEEISIQQDEDLVIFSETSLDGWLQGQNSRGETGLFPASYVEIVRSGISTNHADYSSSPAGSPGAQVSLYNSPSVASPARSGGGSGFLSNQGSFEEDDDDDWDDWDDGCTVVEEPRAGGLGTNGHPPLNLSYPGAYPSQHMAFRPKPPLERQDSLASAKRGSVVGRNLNRFSCFVRSGVEAFILGDVPMMAKIAETYSIEMGPRGPQWKANPHPFACSVEDPTKQTKFKGIKSYISYKLTPTHAASPVYRRYKHFDWLYNRLLHKFTVISVPHLPEKQATGRFEEDFIEKRKRRLILWMDHMTSHPVLSQYEGFQHFLSCLDDKQWKMGKRRAEKDEMVGASFLLTFQIPTEHQDLQDVEDRVDTFKAFSKKMDDSVLQLSTVASELVRKHVGGFRKEFQKLGSAFQAISHSFQMDPPFCSEALNSAISHTGRTYEAIGEMFAEQPKNDLFQMLDTLSLYQGLLSNFPDIIHLQKGAFAKVKESQRMSDEGRMVQDEADGIRRRCRVVGFALQAEMNHFHQRRELDFKHMMQNYLRQQILFYQRVGQQLEKTLRMYDNL

SEQ ID NO: 4 - protein, 972 aa, Homo sapiens
  misc_feature: Incyte ID No 3418524CD1
MYGSARSVGKVEPSSQSPGRSPRLLRSPRLGHRRTNSTGGSSGSSVGGGSGKTLSMENIQSLNAAYATSGPMYLSDHENVGSETPKSTMTLGRSGGRLPYGVRMTAMGSSPNIASSGVASDTIAFGEHHLPPVSMASTVPHSLRQARDNTIMDLQTQLKEVLRENDLLRKDVEVKESKLSSSMNSIKTFWSPELKKERALRKDEASKITIWKEQYRVVQEENQHMQMTIQALQDELRIQRDLNQLFQQDSSSRTGEPCVAELTEENFQRLHAEHERQAKELFLLRKTLEEMELRIETQKQTLNARDESIKKLLEMLQSKGLSAKATEEDHERTRRLAEAEMHVHHLESLLEQKEKENSMLREEMHRRFENAPDSAKTKALQTVIEMKDSKISSMERGLRDLEEEIQMLKSNGALSTEEREEEMKQMEVYRSHSKFMKNKVEQLKEELSSKEAQWEELKKKAAGLQAEIGQVKQELSRKDTELLALQTKLETLTNQFSDSKQHIEVLKESLTAKEQRAAILQTEVDALRLRLEEKETMLNKKTKQIQDMAEEKGTQAGEIHDLKDMLDVKERKVNVLQKKIENLQEQLRDKEKQMSSLKERVKSLQADTTNTDTALTTLEEALAEKERTIERLKEQRDRDEREKQEEIDNYKKDLKDLKEKVSLLQGDLSEKEASLLDLKEHASSLASSGLKKDSRLKTLEIALEQKKEECLKMESQLKKAHEAALEARASPEMSDRIQHLEREITRYKDESSKAQAEVDRLLEILKEVENEKNDKDKKIAELERQVKDQNKKVANLKHKEQVEKKKSAQMLEEARRREDNLNDSSQQLQVEELLMAMEKVKQELESMKAKLSSTQQSLAEKETHLTNLRAERRKHLEEVLEMKQEALLAAISEKDANIALLELSSSKKKTQEEVAALKREKDRLVQQLKQQTQNRMKLMADNYEDDHFKSSHSNQTNHKPSPDQDEEEGIWA

SEQ ID NO: 5 - protein, 189 aa, Homo sapiens
  misc_feature: Incyte ID No 7490407CD1
MQTIKCVVVGDEAIGKTCLLISYTTNVFPEEYIPTVFDNYSVQTSVDGQIISLNTWDTAGQEEYDDCEHSPNPRSIFVICFSTGNPSSYANVRHKWHPEVSHHCPNVPVLLVGTKRDLWSNLETVKKLKEQSLVPTTPQQGTSLAKQLGAVKYLEYSALMQDGVHEVFLEAVRAVLYPATKNTKKYILL

SEQ ID NO: 6 - protein, 1935 aa, Homo sapiens
  misc_feature: Incyte ID No 700648CD1
MRKLFGGPGSRRPSADSESPGTPSPDGAAWEPPARESRQPPTPPPRTCFPLAGLRSARPLTGPETEGRLRRPQQQQERAQRPADGLHSWHIFSQPQAGARASCSSSSIAASYPVSRSRAASSSEEEEEGPPQLPGAQSPAYHGGHSSGSDDDRDGEGGHRWGGRPGLRPGSSLLDQDCRPDSDGLNLSSMNSAGVSGSPEPPTSPRAPREEGLREWGSGSPPCVPGPQEGLRPMSDSVGGAFRVAKVSFPSYLASPAGSRGSSRYSSTETLKDDDLWSSRGSGGWGVYRSPSFGAGEGLLRSQARTRAKGPGGTSRALRDGGFEPEKSRQRKSLSNPDIASETLTLLSFLRSDLSELRVRKPGGSSGDRGSNPLDGRDSPSAGGPVGQLEPIPIPAPASPGTRPTLKDLTATLRRAKSFTCSEKPMARRLPRTSALKSSSSELLLTGPGAEEDPLPLIVQDQYVQEARQVFEKIQRMGAQQDDGSDAPPGSPDWAGDVTRGQRSQEELSGPESSLTDEGIGADPEPPVAAFCGLGTTGMWRPLSSSSAQTNHHGPGTEDSLGGWALVSPETPPTPGALRRRRKVPPSGSGGSELSNGEAGEAYRSLSDPIPQRHRAATSEEPTGFSVDSNLLGSLSPKTGLPATSAMDEGLTSGHSDWSVGSEESKGYQEVIQSIVQGPGTLGRVVDDRIAGKAPKKKSLSDPSRRGELAGPGFEGPGGEPIREVEPMLPPSSSEPILVEQRAEPEEPGATRSRAQSERALPEALPPPATAHRNFHLDPKLADILSPRLIRRGSKKRPARSSHQELRRDEGSQDQTGSLSRARPSSRHVRHASVPATFMPIVVPEPPTSVGPPVAVPEPIGFPTRAHPTLQAPSLEDVTKQYMLNLHSGEVPAPVPVDMPCLPLAAPPSAEAKPPEAARPADEPTPASKCCSKPQVDMRKHVAMTLLDTEQSYVESLRTLMQGYMQPLKQPENSVLCDPSLVDEIFDQIPELLEHHEQFLEQVRHCMQTWHAQQKVGALLVQSFSKDVLVNIYSAYIDNFLNAKDAVRVAKEARPAFLKFLEQSMRENKEKQALSDLMIKPVQRIPRYELLVKDLLKHTPEDHPDHPLLLEAQRNIKQVAERINKGVRSAEEAERHARVLQEIEAHIEGMEDLQAPLRRFLRQEMVIEVKAIGGKKDRSLFLFTDLIVCTTLKRKSGSLRRSSMSLYTAASVIDTASKYKMLWKLPLEDADIIKGASQATNRENIQKAISRLDEDLTTLGQMSKLSESLGFPHQSLDDALRDLSAAMHRDLSEKQALCYALSFPPTKLELCATRPEGTDSYIFEFPHPDARLGFEQAFDEAKRKLASSKSCLDPEFLKAIPIMKTRSGMQFSCAAPTLNSCPEPSPEVWVCNSDGYVGQVCLLSLRAEPDVEACIAVCSARILCIGAVPGLQPRCHREPPPSLRSPPETAPEPAGPELDVEAAADEEAATLAEPGPQPCLHISIAGSGLEMTPGLGEGDPRPELVPFDSDSDDESSPSPSGTLQSQASRSTISSSFGNEETPSSKEATAETTSSEEEQEPGFLPLSGSFGPGGPCGTSPMDGRALRRSSHGSFTRGSLEDLLSVDPEAYQSSVWLGTEDGCVHVYQSSDSIRDRRNSMKLQHAASVTCILYLNNQVFVSLANGELVVYQREAGHFWDPQNFKSVTLGTQGSPITKMVSVGGRLWCGCQNRVLVLSPDTLQLEHMFYVGQDSSRCVACMVDSSLGVWVTLKGSAHVCLYHPDTFEQLAEVDVTPPVHRMLAGSDAIIRQHKAACLRITALLVCEELLWVGTSAGVVLTMPTSPGTVSCPRAPLSPTGLGQGHTGHVRFLAAVQLPDGFNLLCPTPPPPPDTGPEKLPSLEHRDSPWHRGPAPARPKMLVISGGDGYEDFRLSSGGGSSSETVGRDDSTNHLLLWRV

SEQ ID NO: 7 - protein, 567 aa, Homo sapiens
  misc_feature: Incyte ID No 2744459CD1
MPGKPKHLGVPNGRMVLAVSDGELSSTTGPQGQGEGRGSSLSIHSLPSGPSSPFPTEEQPVASWALSFERLLQDPLGLAYFTEFLKKEFSAENVTFWKACERFQQIPASDTQQLAQEARNTYQEFLSSQALSPVNIDRQAWLGEEVLAEPRPDMFRAQQLQIFNLMKFDSYARFVKSPLYRECLLAEAEGRPLREPGSSRLGSPDATRKKPKLKPGKSLPLGVEELGQLPPVEGPGGRPLRKSFRRELGGTANAALRRESQGSLNSSASLDLGFLAFVSSKSESHRKSLGSTEGESESRPGKYCCVYLPDGTASLALARPGLTIRDMLAGICEKRGLSLPDIKVYLVGNEQKALVLDQDCTVLADQEVRLENRITFELELTALERVVRISAKPTKRLQEALQPILEKHGLSPLEVVLHRPGEKQPLDLGKLVSSVAAQRLVLDTLPGVKISKARDKSPCRSQGCPPRTQDKATHPPPASPSSLVKVPSSATGKRQTCDIEGLVELLNRVQSSGAHDQRGLLRKEDLVLPEFLQLPAQGPSSEETPPQTKSAAQPIGGSLNSTTDSAL

SEQ ID NO: 8 - protein, 731 aa, Homo sapiens
  misc_feature: Incyte ID No 60204026CD1
MSGSRQAGSGSAGTSPGSSAASSVTSASSSLSSSPSPPSVAVSAAALVSGGVAQAAGSGGLGGPVRPVLVAPAVSGSGGGAVSTGLSRHSCAARPSAGGGGSSSSLGSGSRKRPLLAPLCNGLINSYEDKSNDFVCPICFDMIEEAYMTKCGHSFCYKCIHQSLEDNNRCPKCNYVVDNIDHLYPNFLVNELILKQKQRFEEKRFKLDHSVSSTNGHRWQIFQDWLGTDQDNLDLANVNLMLELLVQKKKQLEAESHAAQLQILMEFLKVARRNKREQLEQIQKELSVLEEDIKRVEEMSGLYSPVSEDSTVPQFEAPSPSHSSIIDSTEYSQPPGFSGSSQTKKQPWYNSTLASRRKRLTAHFEDLEQCYFSTRMSRISDDSRTASQLDEFQECLSKFTRYNSVRPLATLSYASDLYNGSSIVSSIEFDRDCDYFAIAGVTKKIKVYEYDTVIQDAVDIHYPENEMTCNSKISCISWSSYHKNLLASSDYEGTVILWDGFTGQRSKVYQEHEKRCWSVDFNLMDPKLLASGSDDAKVKLWSTNLDNSVASIEAKANVCCVKFSPSSRYHLAFGCADHCVHYYDLRNTKQPIMVFKGHRKAVSYAKFVSGEEIVSASTDSQLKLWNVGKPYCLRSFKGHINEKNFVGLASNGDYIACGSENNSLYLYYKGLSKTLLTFKFDTVKSVLDKDRKEDDTNEFVSAVCWRALPDGESNVLIAANSQGTIKVLELV

SEQ ID NO: 9 - protein, 654 aa, Homo sapiens
  misc_feature: Incyte ID No 7473835CD1
MKISNEETLQSFKAWRKRWFILRRGQTSSDPDVLEYYKNDGSKKPLRTINLNLCEQLDVDVTLNFNKKEIQKGYMFDIKTSERTFYLVAETREDMNEWVQSICQICGFRQEESTAAVFILGAVAAWPPSSPGDLHGSSSWSAHSSEPSCSHQHLPQEQEPTSEPPVSHCVPPTWPIPAPPGCLRSHQHASQRAEHARRSASFSQGSEAPFIMRRNTAMQNLAQHSGYSVDGVSGHIHGFHSLSKPSQHNAEFRGSTHRIPWSLASHGHTRGSLTGSEADNEGVYPFKAPRSTLFQEFGGHLVNNSGVPATPLSVHQIPRTVTLDKNLYAMVVATPGPIASLPLPKASQAEACQWGSPQQRPLVSESSRWSVAAAIPRRNTLPAVDNSRCHQASSGKYTQHGGGNASRPAESMHEGVCSFLPGRTLVGLSDSIASEGSCVPMNPGSPTLPAVKQAGDDSQGVCIPVGSCLVRFDLLGSPLTELSMHQDLSQGHEVQLPPVNRSLKPNQKDQPTPPNLRNNRVINELSFKPPVTEPWSGTSHTFDSSSSQHPISTQSITNTDSEDSGERYLFPQNPASAFPVSGGTSSSAPPRSTGNIHYAALDFQPSKPSIGSVTSGKKVDYVQVDLEKTQALQKTMHEQMCLRQSSEPPRGAKL

SEQ ID NO: 10 - protein, 80 aa, Homo sapiens
  misc_feature: Incyte ID No 8186336CD1
MGAAAVRWHLCVLLALGTRGRLAGGSGLPGSVDVDECSEGTDDCHIDAIYQNTPKSYKCLCKPGYKGEGKQCEDLVFLET

SEQ ID NO: 11 - protein, 963 aa, Homo sapiens
  misc_feature: Incyte ID No 7493330CD1
MAERGGAGGGPGGAGGGSGQRGSGVAQSPQQPPPQQQQQQPPQQPTPPKLAQATSSSSSTSAAAASSSSSSTSTSMAVAVASGSAPPGGPGPGRTPAPVQMNLYATWEVDRSSSSCVPRLFSLTLKKLVMLKEMDKDLNSVVIAVKLQGSKRILRSNEIVLPASGLVETELQLTFSLQYPHFLKRDANKLQIMLQRRKRYKNRTILGYKTLAVGLINMAEVMQHPNEGALVLGLHSNVKDVSVPVAEIKIYSLSSQPIDHEGIKSKLSDRSPDIDNYSEEEEESFSSEQEGSDDPLHGQDLFYEDEDLRKVKKTRRKLTSTSAITRQPNIKQKFVALLKRFKVSDEVGFGLEHVSREQIREVEEDLDELYDSLEMYNPSDSGPEMEETESILSTPKPKLKPFFEGMSQSSSQTEIGSLNSKGSLGKDTTSPMELAALEKIKSTWIKNQDDSLTETDTLEITDQDMFGDASTSLVVPEKVKTPMKSSKTDLQGSASPSKVEGVHTPRQKRSTPLKERQLSKPLSERTNSSDSERSPDLGHSTQIPRKVVYDQLNQILVSDAALPENVILVNTTDWQGQYVAELLQDQRKPVVCTCSTVEVQAVLSALLTRIQRYCNCNSSMPRPVKVAAVGGQSYLSSILRFFVKSLANKTSDWLGYMRFLIIPLGSHPVAKYLGSVDSKYSSSFLDSGWRDLFSRSEPPVSEQLDVAGRVMQYVNGAATTHQLPVAEAMLTCRHKFPDEDSYQKFIPFIGVVKVGLVEDSPSTAGDGDDSPVVSLTVPSTSPPSSSGLSRDATATPPSSPSMSSALAIVGSPNSPYGDVIGLQVDYWLGHPGERRREGDKRDASSKNTLKSVFRSVQVSRLPHSGEAQLSGTMAMTVVTKEKNKKVPTIFLSKKPREKEVDSKSQVIEGISRLICSAKQQQTMLRVSIDGVEWSDIKFFQLAAQWPTHVKHFPVGLFSGSKAT

SEQ ID NO: 12 - protein, 175 aa, Homo sapiens
  misc_feature: Incyte ID No 7487969CD1
MDNLSSEEIQQRAHQITDESLESTRRILGLAIESQDAGIKTITMLDEQKEQLNRIEEGLDQINKDMRETEKTLTELNKCCGLCVCPCNSITNDAREDEMEENLTQVGSILGNLKDMALNIGNEIDAQNPQIKRITDKADTNRDRIDIANARAKKLIDSKATAVLLYHLFTSVAPP

SEQ ID NO: 13 - protein, 731 aa, Homo sapiens
  misc_feature: Incyte ID No 2655990CD1
MKLRSSHNASKTLNANNMETLIECQSEGDIKEHPLLASCESEDSICQLIEVKKRKKVLSWPFLMRRLSPASDFSGALETDLKASLFDQPLSIICGDSDTLPRPIQDILTILCLKGPSTEGIFRRAANEKARKELKEELNSGDAVDLERLPVHLLAVVFKDFLRSIPRKLLSSDLFEEWMGALEMQDEEDRIEALKQVADKLPRPNLLLLKHLVYVLHLISKNSEVNRMDSSNLAICIGPNMLTLENDQSLSFEAQKDLNNKVKTLVEFLIDNCFEIFGENIPVHSSITSDDSLEHTDSSDVSTLQNDSAYDSNDPDVESNSSSGISSPSRQPQVPMATAAGLDSAGPQDAREVSPEPIVSTVARLKSSLAQPDRRYSEPSMPSSQECLESRVTNQTLTKSEGDFPVPRVGSRLESEEAEDPFPEEVFPAVQGKTKRPVDLKIKNLAPGSVLPRALVLKAFSSSSLDASSDSSPVASPSSPKRNFFSRHQSFTTKTEKGKPSREIKKHSMSFTFAPHKKVLTKNLSAGSGKSQDFTRDHVPRGVRKESQLAGRIVQENGCETHNQTARGFCLRPHALSVDDVFQGADWERPGSPPSYEEAMQGPAARLVASESQTVGSMTVGSMRARMLEAHCLLPPLPPAHHVEDSRHRGSKEPLPGHGLSPLPERWKQSRTVHASGDSLGHVSGPGRPELLPLRTVSESVQRNKRDCLVRRCSQPVFEADQFQYAKESYI

SEQ ID NO: 14 - protein, 727 aa, Homo sapiens
  misc_feature: Incyte ID No 71768694CD1
MACGPPPHTAPAPGLWLSGFGLLRGDHLFLCSAPGPGPPAPEDMVHLRRLQEISVVSAADTPDKKEHLVLVETGRTLYLQGEGRLDFTAWNAAIGGAAGGGGTGLQEQQMSRGDIPIIVDACISFVTQHGLRLEGVYRKGGARARSLRLLAEFRRDARSVKLRPGEHFVEDVTDTLKRFFRELDDPVTSARLLPRWREAAELPQKNQRLEKYKDVIGCLPRVNRRTLATLIGHLYRVQKCAALNQMCTRNLALLFAPSVFQTDGRGEHEVRVLQELIDGYISVFDIDSDQVAQIDLEVSLITTWKDVQLSQAGDLIMEVYIEQQLPDNCVTLKVSPTLTAEELTNQVLEMRGTAAGMDLWVTFEIREHGELERPLHPKEKVLEQALQWCQLPEPCSASLLLKKVPLAQAGCLFTGIRRESPRVGLLRCREEPPRLLGSRFQERFFLLRGRCLLLLKEKKSSKPEREWPLEGAKVYLGIRKKLKPPTPWGFTLILEKMHLYLSCTDEDEMWDWTTSILKAQHDDQQPVVLRRHSSSDLARQKFGTMPLLPIRGDDSGATLLSANQTLPMKSSQGSVEEQEELEEPVYEEPVYEEVGAFPELIQDTSTSFSTTREWTVKPENPLTSQKSLDQPFLSKSSTLGQEERPPEPPPGPPSKSSPQARGSLEEQLLQELSSLILRKGETTAGLGSPSQPSSPQSPSPTGLPTQTPGFPTQPPCTSSPPSSQPLT

SEQ ID NO: 15 - protein, 159 aa, Homo sapiens
  misc_feature: Incyte ID No 5079019CD1
MGVYERLSAEQIKEYKGVFEMFDEEGNGEVKTGELEWLMSLLGINPTKSELASMAKDVDRDKDGDRTIDYEGEWPMGVYHEKAQNQESELRAAFRVFDKEGKGYIDWNTLKYVLMNAGEPLNEVEAEQMMKEADKDGDRTIDYEEFVAMMTGESFKLIQ

SEQ ID NO: 16 - protein, 1356 aa, Homo sapiens
  misc_feature: Incyte ID No 894500CD1
MALNLQLSDTDDNETFDELHIESSDEKTPSDVSLAADTDKSVENLDVLVGFGKSLCGSPEEEEKQVPIPSETRPKTFSFIKQQRVVKRTSSEECVTVIFDAEDGEPIEFSSHQTGVVTVTRNEISINSTPAGPKAEHTELLPQGIACLQPRAAARDYTFFKRSEEDTEKNIPKDNVDNVPRVSTESFSSRTVTQNPQQQKLVKPTHNISCQSNSRSSAPMGIYQKQNLTKIPPRGKSSPQKSKLMEPEATTLLPSSGLATLEKSPALAPGKLSRFMKTESSGPSLNYDQIHTFQNIPPNFRTAPGCPSRRDWVQCPKSQTPGSRSRPAIESSDSGEPPTRDEHCGSGPEAGVKSPSPPPPPGRSVSLLARPSYDYSPAPSSTKSETRVPSETARTPFKSPLLKGTSAPVISSNPATTEVQRKKPSVAFKKPIFTHPMPSPEAVIQTRCPAHAPSSSFTVMALGPPKVSPKRGVPKTSPRQTLGTPQRDIGLQTPRISPSTHEPLEMTSSKSVSPGRKGQLNDSASTPPKPSFLGVNESPSSQVSSSSSSSSPAKSHNSPHGCQSAHEKGLKTRLPVGLKVLMKSPQLLRKSSTVPGKHEKDSLNEASKSSVAVNKSKPEDSKNPASMEITAGERNVTLPDSQAQGSLADGLPLETALQEPLESSIPGSDGRDGVDNRSMRRSLSSSKPHLKPALGMNGAKARSHSFSTHSGDKPSTPPIEGSGKVRTQIITNTAERGNSLTRQNSSTESSPNKAPSAPMLESLPSVGRPSGHPSSGKGSLGSSGSFSSQHGSPSKLPLRIPPKSEGLLIPPGKEDQQAFTQGECPSANVAVLGEPGSDRRSCPPTPTDCPEALQSPGRTQHPSTFETSSTSKLETSGRHPDASATATDAVSSEAPLSPTIEEKVMLCIQENVEKGQVQTKPTSVEAKQKPGPSFASWFGFRKSRLPALSSRKMDISKTKVEKKDAKVLGFGNRQLKSERKKEKKKPELQCETENELIKDTKSADNPDGGLQSKNNRRTPQDIYNQLKIEPRNRHSPVACSTKDTFMTELLNRVDKKAAPQTESGSSNASCRNVLKGSSQGSCLIGSSISTQGNHKKNMKIKADMEVPKDSLVKEANENLQEDEDDAVADSVFQSHIIESNCQMRTLDSGIGTFPLPDSGNRSTGRYLCQPDSPEDAEPLLPLQSALSAVSSMRAQTLEREVPSSTDGQRPADSAIVHSTSDPIMTARGMRPLQSRLPKPASSGKVSSQKQNEAEPRPQTCSSFGYAEDPMASQPLPDWGSEVAATGTQDKAPRMCTYSASGGSNSDSDLDYGDNGFGAGRGQLVKALKSAAPAGKSSEKACAGDNSVKVKERASQL

SEQ ID NO: 17 - protein, 672 aa, Homo sapiens
  misc_feature: Incyte ID No 7497866CD1
MADGSLTGGGLEAAAMAPERTGWAVEQELASLEKGLFQDEDSCSDCSYRDKPGSSLQSFMPEGKTFFPEIFQTNQLLFYERFRAYQDYILADCKASEVQEFTAEFLEKVLEPSGWRAVWHTNVFKVLVEITDVDFAALKAVVRLAEPYLCDSQVSTFTMECMKELLDLKEHRLPLQELWVVFDDSGVFDQTALAIEHVRFFYQNIWRSWDEEEEDEYDYFVRCVEPRLRLHYDILEDRVPSGLIVDYHNLLSQCEESYRKFLNLRSSLSNCNSDSEQENISMVEGLKLYSEMEQLKQKLKLIENPLLRYVFGYQKNSNIQAKGVRSSGQKITHVVSSTMMAGLLRSLLTDRLCQEPGEEEREIQFHSDPLSAINACFEGDTVIVCPGHYVVHGTFSIADSIELEGYGLPDDIVIEKRGKGDTFVDCTGADIKISGIKFVQHDAVEGILIVHRGKTTLENCVLQCETTGVTVRTSAEFLMKNSDLYGAKGAGIEIYPGSQCTLSDNGIHHCKEGILIKDFLDEHYDIPKISMVNNIIHNNEGYGVVLVKPTIFSDLQENAEDGTEENKALKIQTSGEPDVAERVDLEELIECATGKMELCARTDPSEQVEGNCEIVNELIAASTQKGQIKKKRLSELGITQADDNLMSQEMFVGIVGNQFKWNGKGSFGTFLF

SEQ ID NO: 18 - protein, 2937 aa, Homo sapiens
  misc_feature: Incyte ID No 832718CD1
MASEKPGPGPGLEPQPVGLIAVGAAGGGGGGSGGGGTGGSGMGELRGASGSGSVMLPAGMINPSVPIRNIRMKFAVLIGLIQVGEVSNRDIVETVLNLLVGGEFDLEMNFIIQDAESITCMTELLEHCDVTCQAEIWSMFTAILRKSVRNLQTSTEVGLIEQVLLKMSAVDDMIADLLVDMLGVLASYSITVKELKLLFSMLRGESGIWPRHAVKLLSVLNQMPQRHGPDTFFNFPGCSAAAIALPPIAKWPYQNGFTLNTWFRMDPLNNINVDKDKPYLYSFRTSKGVGYSAHFVGNCLIVTSLKSKGKGFQHCVKYDFQPRKWYMISIVHIYNRWRNSEIRCYVNGQLVSYGDMAWHVNTNDSYDKCFLGSSETADANRVFCGQLGAVYVFSEALNPAQIFAIHQLGPGYKSTFKFKSESDIHLAEHHKQVLYDGKLASSIAFTYNAKATDAQLCLESSPKENASIFVHSPHALMLQDVKAIVTHSIHSAIHSIGGIQVLFPLFAQLDNRQLNDSQVETTVCATLLAFLVELLKSSVAMQEQMLGGKGFLVIGYLLEKSSRVHITRAVLEQFLSFAKYLDGLSHGAPLLKQLCDHILFNPAIWIHTPAKVVQLSLYTYLSAEFIGTATIYTTIRRVGTVLQLMHTLKYYYWVINPADSSGITPKGLDGPRPSQKEIISLRAFMLLFLKQLILKDRGVKEDELQSILNYLLTMHEDENIHDVLQLLVALMSEHPASMIPAFDQRNGIRVIYKLLASKSESIWVQALKVLGYFLKHLGHKRKVEIMHTHSLFTLLGERLMLHTNTVTVTTYNTLYEVILTEQVCTQVVHKPHPEPDSTVKIQNPVILKVVATLLKNSTPSAELMEVRRLFLSDMIKLFSNSRENRRCLLQCSVWQDWMFSLGYINPKNSEEQKITEMVYNIFRILLYHAIKYEWGGWRVWVDTLSIAHSKVTYEAHKEYLAKMYEEYQRQEEENIKKGKKGNVSTISGLSSQTTGAKGGMEIREIEDLSQSQSPESETDYPVSTDTRDLLMSTKVSDDILGNSDRPGSGVHVEVHDLLVDIKAEKVEATEVKLDDMDLSPETLVGGENGALVEVESLLDNVYSAAVEKLQNNVHGSVGIIKKNEEKDNGPLITLADEKEDLPNSSTSFLFDKIPKQEEKLLPELSSNHIIPNIQDTQVHLGVSDDLGLLAHMTGSVDLTCTSSIIEEKEFKIHTTSDGMSSISERDLASSTKGLEYAEMTATTLETESSSSKIVPNIDAGSIISDTERSDDGKESGKEIRKIQTTTTTQAVQGRSITQQDRDLRVDLGFRGMPMTEEQRRQFSPGPRTTMFRIPEFKWSPMHQRLLTDLLFALETDVHVWRSHSTKSVMDFVNSNENIIFVHNTIHLISQMVDNIIIACGGILPLLSAATSPTGSKTELENIEVTQGMSAETAVTFLSRLMAMVDVLVFASSLNFSEIEAEKNMSSGGLMRQCLRLVCCVAVRNCLECRQRQRDRGNKSSHGSSKPQEVPQSVTATAASKTPLENVPGNLSPIKDPDRLLQDVDINRLRAVVFRDVDDSKQAQFLALAVVYFISVLMVSKYRDILEPQRETTRTGSQPGRNIRQEINSPTSTETPAAFPDTIKEKETPTPGEDIQVESSIPHTDSGIGEEQVASILNGAELETSTGPDAMSELLSTLSSEVKKSQESLTENPSETLKPATSISSISQTKGINVKEILKSLVAAPVEIAECGPEPIPYPDPALKRETQAILPMQFHSFDRSVVVPVKKPPPGSLAVTTVGATTAGSGLPTGSTSNIFAATGATPKSMINTTGAVDSGSSSSSSSSSFVNGATSKNLPAVQTVAPMPEDSAENMSITAKLERALEKVAPLLREIFVDFAPFLSRTLLGSHGQELLIEGLVCMKSSTSVVELVMLLCSQEWQNSIQKNAGLAFIELINEGRLLCHAMKDHIVRVANEAEFILNRQRAEDVHKHAEFESQCAQYAADRREEEKMCDHLISAAKHRDHVTANQLKQKILNILTNKHGAWGAVSHSQLHDFWRLDYWEDDLRRRRRFVRNAFGSTHAEALLKAAIEYGTEEDVVKSKKTFRSQAIVNQNAETELMLEGDDDAVSLLQEKEIDNLAGPVVLSTPAQLIAPVVVAKGTLSITTTEIYFEVDEDDSAFKKIDTKVLAYTEGLHGKWMFSEIRAVFSRRYLLQNTALEVFMANRTSVMFNFPDQATVKKVVYSLPRVGVGTSYGLPQARRISLATPRQLYKSSNMTQRWQRREISNFEYLMFLNTIAGRTYNDLNQYPVFPWVLTNYESEELDLTLPGNFRDLSKPIGALNPKRAVFYAERYETWEDDQSPPYHYNTHYSTATSTLSWLVRIEPFTTFFLNANDGKFDHPDRTFSSVARSWRTSQRDTSDVKELIPEFYYLPEMFVNSNGYNLGVREDEVVVNDVDLPPWAKKPEDFVRINRMALESEFVSCQLHQWIDLIFGYKQRGPEAVRALNVFHYLTYEGSVNLDSITDPVLREIPEAYFIRDPHTFLLTKDFIKAMEAQIQNFGQTPSQLLIEPHPPRSSAMHLCFLPQSPLMFKDQMQQDVIMVLKFPSNSPVTHVAANTLPHLTIPAVVTVTCSRLFAVNRWHNTVGLRGAPGYSLDQAHHLPIEMDPLIANNSGVNKRQITDLVDQSIQINAHCFVVTADNRYILICGFWDKSFRVYSTETGKLTQIVFGHWDVVTCLARSESYIGGDCYIVSGSRDATLLLWYWSGRHHIIGDNPNSSDYPAPRAVLTGHDHEVVCVSVCAELGLVISGAKEGPCLVHTITGDLLRALEGPENCLFPRLISVSSEGHCIIYYERGRFSNFSINGKLLAQMEINDSTRAILLSSDGQNLVTGGDNGVVEVWQACDFKQLYIYPGCDAGIRAMDLSHDQRTLITGMASGSIVAFNIDFNRWHYEHQNRY

SEQ ID NO: 19 - protein, 2969 aa, Homo sapiens
  misc_feature: Incyte ID No 7497717CD1
MASEKPGPGPGLEPQPVGLIAVGAAGGGGGGSGGGGTGGSGMGELRGASGSGSVMLPAGMINPSVPIRNIRMKFAVLIGLIQVGEVSNRDIVETVLNLLVGGEFDLEMNFIIQDAESITCMTELLEHCDVTCQAEIWSMFTAILRKSVRNLQTSTEVGLIEQVLLKMSAVDDMIADLLVDMLGVLASYSITVKELKLLFSMLRGESGIWPRHAVKLLSVLNQMPQRHGPDTFFNFPGCSAAAIALPPIAKWPYQNGFTLNTWFRMDPLNNINVDKDKPYLYSFRTSKGVGYSAHFVGNCLIVTSLKSKGKGFQHCVKYDFQPRKWYMISIVHIYNRWRNSEIRCYVNGQLVSYGDMAWHVNTNDSYDKCFLGSSETADANRVFCGQLGAVYVFSEALNPAQIFAIHQLGPGYKSTFKFKSESDIHLAEHHKQVLYDGKLASSIAFTYNAKATDAQLCLESSPKENASIFVHSPHALMLQDVKAIVTHSIHSAIHSIGGIQVLFPLFAQLDNRQLNDSQVETTVCATLLAFLVELLKSSVAMQEQMLGGKGFLVIGYLLEKSSRVHITRAVLEQFLSFAKYLDGLSHGAPLLKQLCDHILFNPAIWIHTPAKVVQLSLYTYLSAEFIGTATIYTTIRRVGTVLQLMHTLKYYYWVINPADSSGITPKGLDGPRPSQKEIISLRAFMLLFLKQLILKDRGVKEDELQSILNYLLTMHEDENIHDVLQLLVALMSEHPASMIPAFDQRNGIRVIYKLLASKSESIWVQALKVLGYFLKHLGHKRKVEIMHTHSLFTLLGERLMLHTNTVTVTTYNTLYEVILTEQVCTQVVHKPHPEPDSTVKIQNPVILKVVATLLKNSTPSAELMEVRRLFLSDMIKLFSNSRENRRCLLQCSVWQDWMFSLGYINPKNSEEQKITEMVYNIFRILLYHAIKYEWGGWRVWVDTLSIAHSKVTYEAHKEYLAKMYEEYQRQEEENIKKGKKGNVSTISGLSSQTTGAKGGMEIREIEDLSQSQSPESETDYPVSTDTRDLLMSTKVSDDILGNSDRPGSGVHVEVHDLLVDIKAEKVEATEVKLDDMDLSPETLVGGENGALVEVESLLDNVYSAAVEKLQNNVHGSVGIIKKNEEKDNGPLITLADEKEDLPNSSTSFLFDKIPKQEEKLLPELSSNHIIPNIQDTQVHLGVSDDLGLLAHMTGSVDLTCTSSIIEEKEFKIHTTSDGMSSISERDLASSTKGLEYAEMTATTLETESSSSKIVPNIDAGSIISDTERSDDGKESGKEIRKIQTTTTTQAVQGRSITQQDRDLRVDLGFRGMPMTEEQRRQFSPGPRTTMFRIPEFKWSPMHQRLLTDLLFALETDVHVWRSHSTKSVMDFVNSNENIIFVHNTIHLISQMVDNIIIACGGILPLLSAATSPTGSKTELENIEVTQGMSAETAVTFLSRLMAMVDVLVFASSLNFSEIEAEKNMSSGGLMRQCLRLVCCVAVRNCLECRQRQRDRGNKSSHGSSKPQEVPQSVTATAASKTPLENVPGNLSPIKDPDRLLQDVDINRLRAVVFRDVDDSKQAQFLALAVVYFISVLMVSKYRDILEPQRETTRTGSQPGRNIRQEINSPTSTVVVIPSIPHPSLNHGFLAKLIPEQSFGHSFYKETPAAFPDTIKEKETPTPGEDIQVESSIPHTDSGIGEEQVASILNGAELETSTGPDAMSELLSTLSSEVKKSQESLTENPSETLKPATSISSISQTKGINVKEILKSLVAAPVEIAECGPEPIPYPDPALKRETQAILPMQFHSFDRSVVVPVKKPPPGSLAVTTVGATTAGSGLPTGSTSNIFAATGATPKSMINTTGAVDSGSSSSSSSSSFVNGATSKNLPAVQTVAPMPEDSAENMSITAKLERALEKVAPLLREIFVDFAPFLSRTLLGSHGQELLIEGLVCMKSSTSVVELVMLLCSQEWQNSIQKNAGLAFIELINEGRLLCHAMKDHIVRVANEAEFILNRQRAEDVHKHAEFESQCAQYAADRREEEKMCDHLISAAKHRDHVTANQLKQKILNILTNKHGAWGAVSHSQLHDFWRLDYWEDDLRRRRRFVRNAFGSTHAEALLKAAIEYGTEEDVVKSKKTFRSQAIVNQNAETELMLEGDDDAVSLLQEKEIDNLAGPVVLSTPAQLIAPVVVAKGTLSITTTEIYFEVDEDDSAFKKIDTKVLAYTEGLHGKWMFSEIRAVFSRRYLLQNTALEVFMANRTSVMFNFPDQATVKKVVYSLPRVGVGTSYGLPQARRISLATPRQLYKSSNMTQRWQRREISNFEYLMFLNTIAGRTYNDLNQYPVFPWVLTNYESEELDLTLPGNFRDLSKPIGALNPKRAVFYAERYETWEDDQSPPYHYNTHYSTATSTLSWLVRIEPFTTFFLNANDGKFDHPDRTFSSVARSWRTSQRDTSDVKELIPEFYYLPEMFVNSNGYNLGVREDEVVVNDVDLPPWAKKPEDFVRINRMALESEFVSCQLHQWIDLIFGYKQRGPEAVRALNVFHYLTYEGSVNLDSITDPVLREIPEAYFIRDPHTFLLTKDFIKAMEAQIQNFGQTPSQLLIEPHPPRSSAMHLCFLPQSPLMFKDQMQQDVIMVLKFPSNSPVTHVAANTLPHLTIPAVVTVTCSRLFAVNRWHNTVGLRGAPGYSLDQAHHLPIEMDPLIANNSGVNKRQITDLVDQSIQINAHCFVVTADNRYILICGFWDKSFRVYSTETGKLTQIVFGHWDVVTCLARSESYIGGDCYIVSGSRDATLLLWYWSGRHHIIGDNPNSSDYPAPRAVLTGHDHEVVCVSVCAELGLVISGAKEGPCLVHTITGDLLRALEGPENCLFPRLISVSSEGHCIIYYERGRFSNFSINGKLLAQMEINDSTRAILLSSDGQNLVTGGDNGVVEVWQACDFKQLYIYPGCDAGIRAMDLSHDQRTLITGMASGSIVAFNIDFNRWHYEHQNRY

SEQ ID NO: 20 - protein, 616 aa, Homo sapiens
  misc_feature: Incyte ID No 7506420CD1
MADGSLTGGGLEAAAMAPERTGWAVEQELASLEKADCKASEVQEFTAEFLEKVLEPSGWRAVWHTNVFKVLVEITDVDFAALKAVVRLAEPYLCDSQVSTFTMECMKELLDLKEHRLPLQELWVVFDDSGVFDQTALAIEHVRFFYQNIWRSWDEEEEDEYDYFVRCVEPRLRLHYDILEDRVPSGLIVDYHNLLSQCEESYRKFLNLRSSLSNCNSDSEQENISMVEGLKLYSEMEQLKQKLKLIENPLLRYVFGYQKNSNIQAKGVRSSGQKITHVVSSTMMAGLLRSLLTDRLCQEPGEEEREIQFHSDPLSAINACFEGDTVIVCPGHYVVHGTFSIADSIELEGYGLPDDIVIEKRGKGDTFVDCTGADIKISGIKFVQHDAVEGILIVHRGKTTLENCVLQCETTGVTVRTSAEFLMKNSDLYGAKGAGIEIYPGSQCTLSDNGIHHCKEGILIKDFLDEHYDIPKISMVNNIIHNNEGYGVVLVKPTIFSDLQENAEDGTEENKALKIQTSGEPDVAERVDLEELIECATGKMELCARTDPSEQVEGNCEIVNELIAASTQKGQIKKKRLSELGITQADDNLMSQEMFVGIVGNQFKWNGKGSFGTFLF

SEQ ID NO: 21 - DNA, 901 bp, Homo sapiens
  misc_feature: Incyte ID No 7488243CB1
tcagtttccagtctgtggatggtgaggtggcacggacagcggccctggtgccccacactccactgcaagctgcctgcctgtctgccgtgccccaagtccacagcttcccatctcagcacccagctgccactgccctctgtggcccagtgagcagcccagatgcaggccatcaagcgtgtggtggtgggagacagagctgcaggtaaaacttgcctattgactggttatacaaccaactcatttcctggagactatatcctcactgtctttgacaactgttctgccaatgttatggtacttggaaaatcagtgtatctgggcttatggaatacaactggacaagaaactatgccccctatcctatctgcacacagacgtgctcttaatttgcttttcccttggagtcccacatcatttgaaactgctggtgcaaagtggtatcctaaagtgtggcaccactactttccaaacacttttgtcatccttgtgggaattgaacttgatgttagggagcctttgtacactttgctgagaaaatggtggcaccttcatactcaatgccaagtttttggtacagatttaattttcccataaaaccattttgaaccaagcagtaattttaaggtgtaagagtttagactctaaaacgacaagctcttttttttttttggagccaaggtcttgctctgtcgcccaggctgggcttgaactcctggtctcaagtgatcctctcaccttagcctcccagaatatggggattataggtgtaagccaccacacctggctaacaagccttcttaaacgcttatttttcaaagcccctattcttgtttagtttaagagttgccaacctaccttcagaactaaattgttttcttgtgctaaggacactgagcactaa

SEQ ID NO: 22 - DNA, 4064 bp, Homo sapiens
  misc_feature: Incyte ID No 1966295CB1
ggcgccccagcccctggccaagcctctgctgtcatttctcctccctcctctcagtctgcagctgcgggacgggccgggctcctcagtttctgctgtgttgtgaccccacgaggcgctcagcacccagggaaggcgcgtgtgtccccgatgctggctcctccctgagccccgacggctctcgaggttctgagcctgtggcctgcacagggaacttcctctccgactgcatttatgcctctgtggatgtgaaggctatttctagaaatctcttcctttgcagaaacacccgaaaccctcctgccaggaagaccagggcctgggaagagggtcgctctccggccattctcccctcaccctcctcaccttcctcacatcctgtgccctgggggaccagcagctgcttccacccagaacaagcgggagcctgtgtcaggaaagcatgtcagagcagagctgccagatgtccgaactgcggctcctcctcctgggaaaatgccgctcgggaaaaagtgccacaggaaatgccattctgggcaaacatgtgttcaagtccaagttcagtgatcagacagtgatcaaaatgtgccagagagagagttgggtcctgagagaaaggaaggttgtggtaattgacacccctgaccttttctcctcaatagcttgtgctgaagacaagcaacgcaacatccaacactgcttggagctctctgctcccagcctccatgctctgctcttggtaattgccatcggccatttcacaagggaggatgaggaaacagccaagggcatccaacaagtgtttggagctgaagccaggaggcacatcattattgtcttcactcggaaggatgatttgggggatgacttgctgcaagatttcattgaaaaaaacaaacctctcaagcagttggttcaagactatgagggccgatactgcattttcaacaacaagaccaatagtaaggatgagcagatcacccaggtgttggagctccttcgcaaggttgagtctttggtgaatacgaacggaggaccctatcatgtgaacttcaaaactgaaggcagcaggtttcaagattgtgtgaatgaagctgcatctcaagagggagacaagccacagggcccaagggaaaggcagctgcagtccacaggacccgagcagaatccggggacatcagaactgacagtcctccttgtggggaaacgcggtgctggaaaaagtgcagcaggaaacagcattctggggaggcaggcctttcagaccggatttagtgagcagtcagtaacccagagcttcttgtctgagagcagaagctggagaaaaaagaaagtttcgatcattgatgctccggacatctcatctttaaagaacattgactcagaagttagaaaacacatctgtacaggcccccatgccttcctgctggtgacaccactgggcttttacactaagaatgatgaggcagtgctgagcaccatccaaaacaattttggagaaaaattctttgagtacatgatcatacttcttaccaggaaagaagatttaggggatcaggatctagatacgttcttaagaaacagcaataaagctctctatggtctcatccagaagtgtaaaaacagatatagtgccttcaactaccgggcaacaggagaagaagagcaaaggcaggcggacgagctcctggaaaaaattgagagcatggtgcatcagaatgggaacaagcattgtgttttcagagaaaaagaaaccctgaacattgtccttgtggggagaagcgggactgggaagagtgcgaccgggaactctatcctggggagcctcgtcttcacctctcggctccgggcccagccagtcaccaagaccagccagagtggcaggaggacatgggacggacaggaggtggtggttgtggacactccttccttcaaccagatgctggatgtcgaaaaggacccatcccggttagaagaggaggtcaagcgctgtttgtcctgctgtgaaaaaggggacacattttttgtcctggtgttccagctgggacgattcactgaagaggacaaaacagctgtggcgaaactggaggccatctttggagcagactttacgaaatacgcgattatgctgttcacccggaaggaagacctaggggcggggaatttggaagacttcatgaagaactcagataacaaagcccttcggcgcatttttaaaaagtgtgggcggcgagtttgtgcttttaacaacaaagaaacaggccaggcccaggaaacccaggtgaaagctcttttaacaaaggtcaatgatctgagaaaagaaagtgggtggtccgggtatccccatacacaggagaacgtcagcaaactaattaaaaatgtccaggaaatgtcccaagccgaaaaactccttaaaaatttaataggtattttacaataggtagccgaagtgcctggggtctcttcaattagagacaccctcaggttggggggaggggcggggcatggtacaacctgtgggaagggaagcgggttcatggctttgagggcctgagaggcaaatgcatcccgccttgtgatgtatcagctatttgtagataaataaattgcaggtgggggcgaatagtagggtattataaaggagaaagaagatacaaggtggggaaatctggaaaaagatttcagacattaggctgataataagtggtagaatcaagtcacaagaatcacctcacttgtgtaggtaggttggaatcaggatagacactgtgatcaaggctgaggccacctgggatgagaatatagatagtcgtacaacaacccaggggatccagatacatgagaccagggcagcgttcagcacactcctgggcatggcggaagcaggaagcagactccagagggcctccctgttgtccagggccgttggctccctgagctctcctgtctcctttgggatgcgacttagggctagagtgttcttttcttcacacgttagccattccttacggctcttcacgttccacgcctagatgtgaagtgggacatagtcttccattgctggctgtccctgaggaaacagacatgagtcttggaggttctgaatgattttattgctaattatcacagtgacatagactagaacagaaattagaagcatagtaagattgccaaaatcagagaatcttgcaaagttctgtaattctaagtgttgttctagatttcctctagagaaggttattagaatctccattgcgtttctctttctccttctctttcccttgaggttaggaaacaggttaaaactcagagaactccaataataatggtttaaaaacatcaggggcttcctgtatctcctgtcaggaagccgaggaataagcaggctggggctggtgggcgtcaacatcccagttttgttctatctttctgtcccacctgcactgggatgtggcttctacactcgaatttgcttcttggtttcaagacagtggtgttctttccatagctgagcagattattttgagaggtgggtgatatgtgagagagaaatctggaaccttcttctgggtagatacaggataagatagatacagggtaaaatgttgagcactttgtacatgctttgagagcataatctttgtcatctgtttttttcccctagacaatatcaggttaccgtcaacattaatccatttaaaaggacatggactgttgccattaatacttttggattccatataacccttaacacaataacttctagaaaatgtgtgtgccgtagacacaaagaagggaacaatagacaccagggtatacttgagggtggagggtggcaggagggtgaagattgaaaagctgcctattgcgtgttatgctggtttcctgggtgacaaaattacctgtacaccacacccctgtgacacacaacttactcatgtaacaaacctgtgcatgtaccccttgaacctgaaataaaaattggaaagtaaaaaaaagtgtgtaaatatatttagttgaatcaaataataaagatgaatcattatgaattcacaaaaaaaaaaaaa

SEQ ID NO: 23 - DNA, 3407 bp, Homo sapiens
  misc_feature: Incyte ID No 113399CB1
agactgtggacatgagccctccctgctcacaagcatatgcccggagacctgatagggcagtttctgggccatggacattgctttgaagagggggagactggacagcatctgtgggtgctgagaccccaccttaggacctgagagattgaactgtgtaagcgccattcagctgcgagtgcattcttggactgccttgtgagcatccccggtctgggcaggaccctctccttcccatctttctataccacccagcccagccatggcactgaaaggccgagccctctatgactttcacagtgagaacaaggaggaaatcagcatccagcaggatgaggacctggtcatctttagcgagacctcactggatggctggctgcagggccagaacagccgtggggagacagggctctttcctgcctcttatgtggagatcgtccgttctggcatcagcaccaaccatgctgactactccagcagccctgcaggctctcccggagcccaggtgagcttgtacaacagccccagtgtggccagcccagctaggagtggtgggggcagtggcttcctctcaaaccagggtagctttgaggaggatgatgatgatgactgggatgactgggacgacggatgcacagtggtggaggagccacgggctggtgggctgggcaccaacgggcaccctcccctcaacctctcctaccctggtgcctaccccagccagcacatggccttccggcccaagccaccactggagcggcaggacagcctggcatctgccaagcgaggcagtgtggtgggccgtaacctcaaccgtttctcatgctttgtgcgttctggagtggaggccttcatcctgggtgatgtgcccatgatggccaagatcgctgagacatactccattgaaatgggccctcgtggcccccagtggaaggccaatccccacccatttgcctgctctgtggaggaccccacaaaacagaccaaattcaagggcatcaaaagctacatctcctacaagctcacacccacccatgctgcctcacccgtctaccggcgctacaaacactttgactggctctataaccgcctgctacacaagttcactgtcatctcggtgccccacctgcctgagaagcaggccactggccgcttcgaggaggacttcatcgaaaagcggaagcggagactcatcctctggatggaccacatgaccagccaccctgtgctctcccagtacgaaggcttccagcatttcctcagctgcctggatgacaagcagtggaagatgggcaaacgccgggcggagaaggatgagatggtgggtgccagcttcctgctcaccttccagatccccaccgagcaccaggacttgcaggacgtggaagatcgcgtggacactttcaaggccttcagtaagaagatggacgacagcgtcctgcagctcagcactgtggcatcagagctggtgcgtaaacatgtggggggcttccgcaaggaattccagaagctgggcagtgccttccaggccatcagtcattccttccagatggacccccccttttgctctgaggccctcaacagtgccatttctcacacgggccgtacctatgaagccatcggggagatgtttgctgagcagcccaagaatgacctcttccagatgctggacacactgtctctctaccagggcctgctctccaacttccctgacatcatccatctacaaaaaggcgccttcgccaaggtgaaggagagccaacgcatgagtgacgagggccgcatggtgcaggacgaggcagacggcattcgcaggcgctgccgcgtggtgggtttcgccctgcaggccgagatgaaccacttccaccagcgccgtgagctcgacttcaagcacatgatgcagaactacttgcgccagcagatcctcttctaccagcgggtgggccagcagctggagaagaccctgcgcatgtatgacaacctctgaccgcgtgtgcctgggccccctccttcccctgggcctggtcactgcagtgtaccccactttcccgacctccctataccagcagtgactgggggaggggtcagcggtgggggagataagcggcctgtcctgcctcctgggagaaggagctttcaaggagtcatgggtgcccctgggaaattccccactccttagaagtggggcacagcaggggtgagaatagagtcaggagccctcgaggccaaggcctgggctgccggtcagtcagtgaaggtcaggccagggtctcagcctcccctagagcctattttgcttgctcacctggccactgctgccttatccattcagcagacaccgaggcctgctgcacccttgggtcggatgctgggcacccagggctgtgacatgcctgctcttcaggagtcctcagtgaaggtcggggtcagacacagacagagtcaatgcagtatgactgatgtttaagtgagggatttctggaagctcatagaagggaccacagcattccactggtcagggaagactccatagagtaggcaacatttgggcagtgttttgaagaatgacaagggcctgccagacagtacatgggggagaaggactttcaggggagaggaacagcatgggcaaagttatggaggcatgcaaacatctccctcttctctcccttactttccaagcaagttaggtacgctttccatggggattctggcctgtgtggtaggaagggatctcccttgctcccatgttgctggctgtccgtacatcaccctgtcccctgcaggagggggctacaggccatctccctcctgtaggcctctgactcccctccacttttgggccctcagcttatctcgggcaggggaccattgcagcatcctcccctcctcggactcaaggtgctgaggtataagccctgggccccagatccctggtgacaccttcctggagaagactctcaaaagtgactgtatatttgagttcaccagcaataactccccacactcgaagcaggtccaaacccaggatctcagggtccttgggctctgtggcactgtcttcccaagatccttcctgttgcacaatgggaaacctaagaggaaaaagacaggggcctgcttgcccagccatgcgagggattccatgcccacctgccctctgtctgcctcgctggaatgtgggcccctgctccccgtcaggttgtgctgtctctgacctatgtttacatccccgaggggtttctgcctcctccccacccaggtcagggtgtggtccagcagcttgctgtggggtgctgacatgtgtcaccactgccccccttgcccccgggggggtcatggtctcctcctggatgctgctccttgaatcttttttcttgataaaccttttacaattaagaaaaaaaaaa

SEQ ID NO: 24 - DNA, 3270 bp, Homo sapiens
  misc_feature: Incyte ID No 3418524CB1
agccgagccgggagcccggggaggcgcgctgcgccccgcggggggctgggggcggggctaggtgtctcgcgcaggtggcggggcgcggtgacgtcgcgggcgcctgggccgtgctgtggcggcggcggcggcggtagtggcggcggcggcggtgcccgggcggcagcggcagcagtagcggcagccctgaggacgatatggtgtaagatacttcttcaagattggacagctggggaccttcttctgattaaccttaaaccaacttgtagccatagagacacctcacaaggttcccatttttgttgttgttgttgttgattttctgctcacacctttcctgaccttgcaaccatgtatggaagtgcccgctctgttgggaaggtggagccgagcagccagagccctgggcgttcacccaggcttctacgttcccctcgcttgggtcaccgtcgaaccaacagtacgggagggagttcgggaagcagtgttggaggtggcagtgggaaaaccctttcaatggaaaatatacaatctttaaatgctgcctatgccacctctggccctatgtatctaagtgaccatgaaaatgtgggttcagaaacacctaaaagcaccatgacacttggccgttctgggggacgtctgccttacggtgttcggatgactgctatgggtagtagccccaatatagctagcagtggggttgctagtgacaccatagcatttggagagcatcacctccctcctgtgagtatggcatccactgtacctcactcccttcgtcaggcgagagataacacaatcatggatctgcagacacagctgaaggaagtattaagagaaaatgatctcttgcggaaggatgtggaagtaaaggagagcaaattgagttcttcaatgaatagcatcaagaccttctggagcccagagctgaagaaggaacgagccctgagaaaagatgaagcttccaaaatcaccatttggaaggaacagtacagagttgtacaggaggaaaaccagcacatgcagatgacaatccaggctctccaggatgaattgcggatccagagggacctgaatcagctgtttcagcaggatagtagcagcaggactggcgaaccttgtgtagcagagctgacagaggagaactttcagaggcttcatgctgagcatgagcggcaggccaaagagctgtttcttcttcgaaagacattggaggaaatggagctgcgtattgagactcaaaagcagaccctaaatgctcgggatgaatccattaagaagcttctggaaatgttgcagagcaaaggactttctgccaaggctaccgaggaagaccatgagagaacaagacgactggcagaggcagagatgcacgttcatcacctagaaagccttttggagcagaaggaaaaagagaacagtatgttgagagaggagatgcatcgaaggtttgagaatgctcctgattctgccaaaacaaaagctctgcaaactgttattgagatgaaggattcaaaaatttcctctatggagcgtgggcttcgagacctggaagaggaaattcagatgctgaaatcgaatggtgctttgagtactgaggaaagggaagaagaaatgaagcaaatggaagtgtatcggagccattctaaatttatgaaaaataaggtagaacaactgaaggaggaactaagttcgaaagaggctcaatgggaggagctgaaaaagaaagcggctggtcttcaggctgagattggccaggtgaaacaggagctgtccagaaaggacacagaactactcgccctgcagacaaagctagaaacactcacaaaccagttctcagatagtaaacagcacattgaagtgttgaaggagtccttgactgctaaggagcagagggctgccatcctgcagactgaggtggatgctctccgattgcgtttggaagagaaggaaaccatgttgaataaaaagacaaaacaaattcaggatatggctgaagagaaggggacacaagctggagagatacatgacctcaaggacatgttggatgtgaaggagcggaaggttaatgttcttcagaagaagattgaaaatcttcaagagcagcttagagacaaggaaaagcagatgagcagcttgaaagaacgggtcaaatccttgcaggctgacaccaccaacactgacactgccttgacaactttggaggaggcccttgcagagaaagagcggacaattgaacgcttaaaggagcagagggacagagatgagcgagagaagcaagaggaaattgataactacaaaaaagatcttaaagacttgaaggaaaaagtcagcctgttgcaaggcgacctttcagagaaagaggcttcacttttggatctgaaagagcatgcttcttctctggcatcctcaggactgaaaaaggactcacggcttaagacactagagattgctttggagcagaagaaggaggagtgtctgaaaatggaatcacaattgaaaaaggcacatgaggcagcattggaagccagagccagtccagagatgagtgaccgaatacagcacttggagagagagatcaccaggtacaaagatgaatctagcaaggcccaggcagaagttgatcgactcttagaaatcttgaaggaggtggaaaatgagaagaatgacaaagataagaagatagctgagttggaaaggcaagtgaaagaccagaataagaaggtagcaaatctgaagcacaaggaacaggtggaaaaaaagaagagtgcacaaatgttagaggaggcgcgacgacgggaggacaatctcaacgacagctctcagcagctacaggtggaggagttactgatggccatggagaaggtaaagcaggaactagaatccatgaaagcaaagctgtcctccacccagcagtctctggcagaaaaggaaactcacttgactaatcttcgggcagagagaaggaaacacttagaggaagttctggagatgaagcaagaagctcttctggctgccattagtgaaaaagacgccaatatagctctcttggagctttcgtcctctaagaagaagacccaagaggaagtggctgccctgaagcgggagaaggatcgtctggtacagcagcttaagcagcagacgcaaaatcgaatgaagctaatggccgacaactacgaggatgaccacttcaaatcctcccattccaatcaaacaaatcacaagccctccccagaccaggatgaggaggagggtatatgggcatag

SEQ ID NO: 25 - DNA, 570 bp, Homo sapiens
  misc_feature: Incyte ID No 7490407CB1
atgcagacaatcaaatgtgtggttgtaggagatgaggctataggtaaaacctgcctcctcatcagttatacaacaaatgtctttcctgaggagtatatccccactgtctttgacaactatagcgtccagacatccgtggatgggcaaatcattagcctgaacacgtgggacactgctggccaagaggagtatgatgactgcgaacactctcctaaccccagaagtatctttgttatttgtttttccactgggaacccatcctcttatgccaatgtgaggcataagtggcacccagaggtctcccatcattgccccaatgtgcctgttctgctggtaggcaccaagagggacctgtggagtaaccttgagacagtgaagaagctgaaggagcagagcctagtacccacgactccccagcaaggcacttccctggctaagcagttgggggctgtgaagtatctggaatattcggccctgatgcaggatggggtgcatgaggtatttttagaagctgtccgggctgtgctttaccctgctacaaagaacaccaagaagtacatcctcttatag

SEQ ID NO: 26 - DNA, 7768 bp, Homo sapiens
  misc_feature: Incyte ID No 700648CB1
accccgagttccaaggaggccacggcagagaccaccagctcagaggaggagcaggagccaggcttcctgccactgtctggctcctttgggcctggtggtccctgcggcaccagcccaatggatgggagagcccttcgccgctccagccacggctccttcacccggggcagccttgaggacctgctgagtgtcgaccctgaggcctaccagagctccgtgtggctgggcactgaggatggctgtgtccacgtgtaccagtcctccgacagcatccgtgaccgcaggaacagcatgaagctccagcatgcggcctctgtgacctgcatcttgtaaggccccagggtggcccttcctatctagactcttctcagccaccgacgccaccccctcggacatgcttccccctggcgggtctgcgttcggcgcggcccctgaccgggccggagaccgaagggaggctgcgccggccgctccgcagcctctcgccgtcggttcgccagctctcccggcgcttcgacgcgccgcgtctggacgacggctccgctgggacccgagacggaggcgtcttacccgcggccgcggaagaagcggccgagggcccagcgcgaggagcctggcccagcgtcaccgagatgcgcaagctcttcggcggtcctggctccaggaggcccagcgccgactctgaatccccaggaacgcccagccccgacggtgccgcgtgggagcctccggctcgggagtcgcggcagccaccgacgccaccccctcggacatgcttccccctggcgggtctgcgttcggcgcggcccctgaccgggccggagaccgaagggaggctgcgccggccgcagcagcaacaggagcgggcgcagcgtccagcggatggtttacattcttggcatatcttctcccaaccgcaggccggggcccgggcctcctgctcctcctcctccatcgccgcctcctatcctgtcagccgcagtcgtgctgccagctccagcgaggaggaagaggagggcccgccgcagctgcctggagcccagagtccggcctaccacggcggccactcctcgggcagtgacgacgaccgagacggtgagggcggccaccgctggggagggaggcccgggctcaggcctggaagctccctattggatcaggactgcaggcctgacagtgatgggttaaatctaagcagcatgaactcagcaggggtttctgggagccctgagcccccaacatctccaagagcccctagagaagaaggactccgggagtggggtagtggctctccgccctgcgtcccaggtccccaggagggacttcggcctatgtctgactctgtgggaggagctttccgtgtggccaaggtgagctttccctcgtacctggccagccccgcaggctcccgcggtagcagccgttattccagcacggagaccctcaaggacgacgacctatggtctagtaggggttctgggggctggggcgtgtaccgctcccctagctttggagctggggaagggctcctgcggtcccaggctcgaacccgtgccaaaggacctggaggcacctctagggcattgagggatggaggatttgagcctgaaaagagtcgacagcggaagtccctgtcaaatccagatatcgcctcagagaccctgacgcttctcagtttcctgcgctcagacctttcagagctgagggtccgaaaacctggtgggagctccggggaccgtggaagcaaccccctagatggcagagactcaccatccgcaggtggccctgtggggcaacttgaacccatacccatcccagccccagcatcacctggcacgcgccccacactcaaggacttgacagccactctgcggagagcaaagtcattcacctgctctgagaagcccatggcccgccgcctgccccgcaccagtgctctgaagtccagctcctccgagctcctgctcacaggccctggtgccgaggaggatccgctgcccctcatcgtccaggaccaatatgtgcaggaggcccgccaggtttttgagaagatccagcgcatgggtgcccaacaagatgatggaagcgatgccccccctggaagccctgactgggcaggggatgtgacccgagggcagcggtcccaggaggagctctcaggccctgagtccagtctgacagatgaaggcattggggcagaccctgagcctcctgttgcagcattttgcggcctgggtaccacagggatgtggcgacctctttcctcatcctcggcccagacgaaccaccatggccctgggactgaggacagtctgggcgggtgggccctggtgtcgcctgagacccctcccacaccaggtgccctccgccgacgacgcaaagtcccaccttcaggttctggtgggagcgaattgagcaatggggaggcaggggaggcctacaggtccctgagtgacccaattcctcagcgccaccgggctgccacctctgaagagcctactgggttctctgtggacagcaacctcctgggctcactgagccccaagacagggctccctgccacctcagccatggatgagggcttgaccagtggtcacagtgactggtctgtgggcagtgaagagagcaagggatatcaggaggttattcagagcatagttcaggggcctggcaccctggggcgtgtggtggacgacaggattgctggcaaagcccccaagaagaaatccctgagtgaccccagccgccgtggggagctggctgggcctggattcgagggccctggaggggagcccatccgagaagttgagcccatgctgcctccatccagcagcgagcccatccttgtagagcagcgggcagagccagaagaacctggtgccaccaggagccgggcacagtctgaaagggccctacctgaggctctgcctccccctgccactgcccaccgaaactttcaccttgaccccaagctggctgacattctgtccccgaggctaatccgccgaggctccaagaagcgcccagctcggagtagtcaccaggagcttcggagagacgagggcagtcaggaccagactggcagcctgtctcgggcccggccctcctccagacacgttcgccatgccagtgtgcccgccacatttatgcctattgtggtgcctgagccaccaacttctgttggtccccctgtggctgtgccagaacccataggcttccctacccgagcccatcccacgttgcaggcaccatcgctcgaggacgtcaccaagcagtacatgctgaacctgcactccggtgaggtccctgccccagtgccagtggacatgccctgcttgcctctggctgcaccgccctctgctgaggccaagccccctgaggcagctcggcctgcagatgagcctacccctgccagcaagtgctgcagcaagccacaggtggacatgcggaagcacgtggccatgaccctgctggacacagagcagtcgtatgtggagtcgctgcgcaccctgatgcagggctacatgcagccgctgaagcagccagagaactccgtgctctgtgacccttcactggtggacgagatcttcgaccagatccccgagctcctggagcaccacgagcaattcctggagcaggttcggcactgcatgcagacctggcatgcccagcagaaggtgggagccctgctcgtccagtcgttctccaaggatgtcctagtaaacatctattctgcctatatcgataacttcctcaatgcaaaggatgctgtgcgtgtggccaaggaggcgaggcctgcctttctcaagttcctagagcaaagcatgcgtgagaacaaggagaagcaggcgctgtctgacctcatgatcaagcctgtgcagcggatcccacgctacgagcttctggtgaaggacctcctgaagcatacacctgaggaccacccggaccatccactcctgctggaggcgcagcggaacatcaagcaggtggctgagcgcatcaacaagggtgtgcggagtgccgaggaggcggagcgccatgcccgtgtgctgcaggagatagaggctcacatcgagggcatggaggatctccaggcccctctgcggcggttcctgagacaggagatggtcattgaagtgaaggcgatcggtggcaagaaggaccggtctctcttcctgttcacggacctcatcgtctgcaccactctgaagcgaaagtcaggctccctgcggcgcagctccatgagcctgtacacggcagccagtgtcattgacacagccagcaagtacaagatgctgtggaagctgccgctggaagacgcagacatcatcaaaggggcatcccaagccaccaatcgggagaacatccagaaggccatcagccgccttgatgaggacctcaccaccctgggccaaatgagcaagctctctgagagccttggtttcccccaccagagcctggacgatgcactgcgggacctctcagctgccatgcaccgggacctgtcggagaagcaggcgctgtgctacgcgctttccttcccgccaaccaagctggagctgtgcgccactcggcccgagggcaccgactcctacatttttgagttccctcaccctgacgcccgccttggttttgaacaggccttcgatgaggccaagaggaagctggcatccagcaaaagctgtctagaccctgagttcctgaaggccatccccatcatgaaaacccgcagtggcatgcagttctcctgtgcggctcccaccctgaacagctgcccggagccctcgcctgaggtatgggtctgcaacagcgacggctacgtgggccaggtgtgcctgctgagcctgcgcgccgagccggacgtggaggcctgcatcgccgtctgttccgcccgcatcctctgcatcggggcggtgcccgggctgcagcctcgctgccaccgggagcctcctccgtcgctgaggagtcctccagagacggcaccggagcccgccgggccggagctggacgtcgaggccgctgcagacgaggaagccgcgacgctcgcggagccggggccgcagccctgccttcacatctccattgcaggctcgggcttggagatgacgccgggcctcggcgagggtgacccccgcccagagctggtgccctttgacagtgactctgacgatgagtcttcgcccagcccctcggggacgctgcagagccaggccagccggtccaccatctcctccagctttggcaatgaggagaccccgagttccaaggaggccacggcagagaccaccagctcagaggaggagcaggagccaggcttcctgccactgtctggctcctttgggcctggtggtccctgcggcaccagcccaatggatgggagagcccttcgccgctccagccacggctccttcacccggggcagccttgaggacctgctgagtgtcgaccctgaggcctaccagagctccgtgtggctgggcactgaggatggctgtgtccacgtgtaccagtcctccgacagcatccgtgaccgcaggaacagcatgaagctccagcatgcggcctctgtgacctgcatcttgtatctgaataaccaggtgtttgtgtctctggccaatggagagcttgtggtctaccaaagggaagcaggccatttctgggacccccagaacttcaaatcagtgaccttgggcacccaggggagccccatcaccaagatggtatctgtgggtgggcggctgtggtgtggctgccagaaccgagtccttgtcctgagccctgacacgctgcagctggagcacatgttttacgtgggtcaggattcaagccgctgcgtggcttgcatggtggactccagcctgggtgtgtgggtgacattgaaaggtagtgcccacgtgtgtctctaccatccagacacctttgagcagctggcagaagtagacgtcactcctcccgtgcacaggatgctggcaggctcggatgccatcatccggcagcacaaggctgcctgtctgcgaatcacagcgctgctggtgtgtgaggagctgctgtgggtgggcaccagtgctggtgtcgtcctcaccatgcccacttcgcccggtactgtcagctgcccacgggcaccactcagtcccacaggcctcggccagggacacaccggccacgtccgcttcttggctgcagtccagctgccagatggcttcaacctgctctgcccaaccccaccacctcccccagacacaggccccgagaagctgccatcactggagcaccgggactccccttggcaccgaggccccgcccctgccaggcctaaaatgctggttatcagtggaggtgatggctatgaggacttccgactcagcagtgggggcggcagcagcagtgagactgtgggtcgagacgacagcacaaaccacctcctcctgtggagggtgtgaccctgtctgccgtggcccaggactcgcccgcccacctgccttcagcctgcttgcctctccctagcccacacgcagactttgaccaggagtatccagccaggggcacacatgtgcctgcgtgggctctgccttgtcttcgcggaagcattcctgatggaacacccactggccagccaggccatggcttctcccgaccctctggctgccccggtgcttccagtcatgatcgggtgggggacatgtgggctgaccaggacctctgaccctggagcttctaccaaagacacagctgggtctggaccccacggggctggggagggccatgtgcaatatttggagggttttctggagggcagcaggaaggctggggaattccccatgtacagtatttatgtttctttttagatgtgtaccttcccaagcacttatttatgcagtgacctggtcacctggggtgggggtgatttgaggaaatgacatgaggaaaagaaacctattcctgccctggggaccaccctgggactctaaccaagccttcctggagggacccatgcgcccctgagccccattccattcatacagacacacacgtacgcacactgcatgtccaaggccctaaacattgcccgttgacataaactttccagggccccagcctgatggggctgccctcagtcctctagatcaagatgctgactattagggggcagtgattgccatctggggacctgtcaggctttgtcatttcccagtttgttggtggtgcctttagtggttccctaatttgggaacactgatggggccttggacagggctttctctcaggtaggagaaatgggcccatgatctcctcacagtcgcccccagtccttggccctgcttccctgtgtctcatgcactggcacatatggtcaccttggagggcagacctaggagcccctctgaccactgaatccgtctccacaccccttctgccaagggaagccccttcaggaaggaccccccaaagctgaggggctgaatgtagccttttcaacagagaaggctcccacttgagagcagcctctacctgaccccctggaccacagagagccactctgaccctcagccccctcgcttcttcagctaaaactccaaaggtttggtttcagatggggtttgttttgttctgtttggttttggttttgtttggggtgggtgggtcattgcggtcttagattatgtttctcttgctaccaaacagtcatgtattaactctctttggatgatgaagtttaaagagtcaataaatagaaacaccagatgaaaaaaaaaaaaaaaaaaaaaaa

SEQ ID NO: 27 - DNA, 2076 bp, Homo sapiens
  misc_feature: Incyte ID No 2744459CB1
ttcaggatgtgagggcccgcaggagccgagtcaggctctctccactgcctgcccgccaccgtgcaagctctggccggcgctgcccacagtccccatggtgggcagcccccgcggcggggacccctgatcggcagcggcatgccagggaagcccaagcacctgggcgtccccaacgggcgcatggttctggctgtgtcagatggagagctgagcagcacgacggggccccagggccagggcgagggccgcggcagctctctcagcatccacagcctccccagtggtcccagcagccccttcccaaccgaggagcagcctgtggccagctgggccctgtccttcgagcggctgttgcaggacccgctgggcctggcttacttcactgagttcctgaagaaggagttcagcgcggaaaacgtgactttctggaaggcctgcgagcgcttccagcagatcccggccagcgatacccagcagctagctcaggaggcccgcaacacctaccaggagttcctgtccagccaggcgctgagcccagtgaacatcgaccgtcaggcctggcttggcgaggaggtgctggccgagccccggccggacatgtttcgggcacagcagcttcagatcttcaacttgatgaagttcgacagctatgcgcgcttcgtcaagtccccgctgtaccgcgagtgcctgctagccgaagccgagggacgccctctgcgggaacctggctcctcgcgcctcggcagccctgacgccacgaggaagaagccgaagctgaagcccgggaagtcgctgccgctgggtgtggaggagttggggcagctgccacccgttgagggtcctgggggccgccctctccgcaagtccttccgccgggagctgggcgggactgcaaacgccgccttgcgccgagagtctcagggctccctcaactcctccgccagcctggaccttggcttcctagccttcgtcagcagcaaatctgagagccaccggaagagccttgggagcacggagggtgaaagtgaaagccggccagggaagtactgctgtgtgtacctgcccgatggcacagcctccttggccctggccagacctggcctcaccatccgagacatgctggcagggatctgtgagaaacgaggcctctctctacctgacatcaaggtctacctggtgggcaatgaacagaaggccctggtcctggatcaggactgcaccgtgctggcggatcaggaagtgcggctggaaaacaggatcaccttcgagctggagctgacggcgctggagcgcgtggtacgaatctcagccaagcccaccaagcggctgcaggaggcgctgcagcccattctggagaagcacggcttgagcccgctagaggtggtgctgcaccggccaggcgagaaacagcctctggatctggggaagctagtgagctcggtggcggcccagagactggttttggacactcttccaggtgtgaagatctccaaagcccgtgacaaatctccctgccgcagccagggctgcccacctagaactcaggataaggccacccatccccctccagcgtcccccagttctctggtgaaggtgcccagtagtgccactggaaagcggcagacctgtgacatcgaaggcctggtggagctgctgaaccgggtgcagagcagcggggcccacgaccagaggggccttctgaggaaagaggacctggtacttccagaatttctgcagctgcccgcccaagggcccagctccgaggagaccccaccacagaccaaatcagcagcccagcccatcgggggatccttgaactccaccaccgactcagccctctgacagctacccaacagtccaggacagctgcatggcacccggcgggccgagcatgccatgggtccgctctgcatgccctgtctgtgccatgagtgtccctggccccttcctgccatgggcaggcccgcaggaagagccggtaggggtggaaaggggactcagatgagacacaccccacagctgccaccgccttgtccctcaacaagctcacctcccccattgtggcctggctgcaag

SEQ ID NO: 28 - DNA, 2818 bp, Homo sapiens
  misc_feature: Incyte ID No 60204026CB1
gccggccacggcgcgccggggttggctgctacagggaccgcggtggcggcggcggcctcgacagcggtagtgccttctactccgcttttttagtttcctccgccccctcccgtgggacgctgttgtgtggctgccttaaaaaaaaacgcaactttattgttcctcagccccacctccggctcggcgggcgtcctcaggatgcactgaggctgaggggaggggaggcggcggagggtcgaggtcgcggtccctctcctccgagcgcccggctggaggggagggagtcacgatgtctggtagccgccaggccgggtcgggctccgctgggacaagccccgggtcctcggcggcctcctcggtgacttccgcctcctcgtctttatcctcttccccgtcgccgccttccgtggcggtttcggcggcagcgctggtgtccggcggggtggcccaggccgccggctcgggcggcctcgggggcccggtgcggcctgtgttggtggcgcccgccgtatcgggtagcggcggcggggcggtgtccacgggcctgtcccggcacagctgcgcggccaggcccagcgccggcggaggaggcagcagctccagcctaggcagcggcagcaggaagcgacctctcctcgcccccctctgcaacgggctcatcaactcctacgaggacaaaagcaacgacttcgtatgccccatctgctttgatatgattgaagaagcatacatgacaaaatgtggccacagcttttgctacaagtgtattcatcagagtttggaggacaataatagatgtcccaagtgtaactatgttgtggacaatattgaccatctgtatcctaatttcttggtgaatgaactcattcttaaacagaagcaaagatttgaggaaaagaggttcaaattggaccactcagtgagtagcaccaatggccacaggtggcagatatttcaagattggttgggaactgaccaagataaccttgatttggccaatgtcaatcttatgttggagttactagtgcagaagaagaaacaactggaagcagaatcacatgcagcccaactacagattcttatggaattcctcaaggttgcaagaagaaataagagagagcaactggaacagatccagaaggagctaagtgttttggaagaggatattaagagagtggaagaaatgagtggcttatactctcctgtcagtgaggatagcacagtgcctcaatttgaagctccttctccatcacacagtagtattattgattccacagaatacagccaacctccaggtttcagtggcagttctcagacaaagaaacagccttggtataatagcacgttagcatcaagacgaaaacgacttactgctcattttgaagacttggagcagtgttacttttctacaaggatgtctcgtatctcagatgacagtcgaactgcaagccagttggatgaatttcaggaatgcttgtccaagtttactcgatataattcagtacgacctttagccacattgtcatatgctagtgatctctataatggttccagtatagtctctagtattgaatttgaccgggattgtgactattttgcgattgctggagttacaaagaagattaaagtctatgaatatgacactgtcatccaggatgcagtggatattcattaccctgagaatgaaatgacctgcaattcgaaaatcagctgtatcagttggagtagttaccataagaacctgttagctagcagtgattatgaaggcactgttattttatgggatggattcacaggacagaggtcaaaggtctatcaggagcatgagaagaggtgttggagtgttgactttaatttgatggatcctaaactcttggcttcaggttctgatgatgcaaaagtgaagctgtggtctaccaatctagacaactcagtggcaagcattgaggcaaaggctaatgtgtgctgtgttaaattcagcccctcttccagataccatttggctttcggctgtgcagatcactgtgtccactactatgatcttcgtaacactaaacagccaatcatggtattcaaaggacaccgtaaagcagtctcttatgcaaagtttgtgagtggtgaggaaattgtctctgcctcaacagacagtcagctaaaactgtggaatgtagggaaaccatactgcctacgttccttcaagggtcatatcaatgaaaaaaactttgtaggcctggcttccaatggagattatatagcttgtggaagtgaaaataactctctctacctgtactataaaggactttctaagactttgctaacttttaagtttgatacagtcaaaagtgttctcgacaaagaccgaaaagaagatgatacaaatgaatttgttagtgctgtgtgctggagggcactaccagatggggagtccaatgtgctgattgctgctaacagtcagggtacaattaaggtgctagaattggtatgaagggttaactcaagtcaaattgtacttgatcctgctgaaatacatctgcagctgacaatgagagaagaaacagaaaatgtcatgtgatgtctctccccaaagtcatcatgggttttggatttgttttgaatatttttttctttttttcttttccctcctttatgacctttgggacattgggaatacccagccaactctccaccatcaatgtaactccatggacattgctgctcttggtggtgttatctaatttttgtgatagggaaacaaattcttttgaataaaaataaataacaaaacaataaaagtttattgagccacaaaaaaaaaaaa

SEQ ID NO: 29 - DNA, 2057 bp, Homo sapiens
  misc_feature: Incyte ID No 7473835CB1
atgaagtctttgggaccttccatgagtcattttagtggtgcagaaggggaacagcctgattagagtgatttcacaaaacaggagaggaggaaatgaagattagcaatgaggaaactcttcagagttttaaggcctggaggaaacgctggtttatcctgcggaggggccagactagcagtgacccagatgttctggaatactacaagaatgatggctccaagaagcccctgcgcaccatcaacctgaacctctgtgagcagctggatgttgatgtgactctgaacttcaacaagaaggagattcagaagggctatatgtttgacatcaagaccagtgagcgtaccttttacctggtggctgagaccagggaggacatgaatgagtgggtccagagcatctgtcagatctgtggcttcaggcaggaggaaagcacagcagctgtgttcatccttggtgccgtggctgcttggccccctagctcaccaggagacctgcatgggagctcatcttggagtgcacatagctctgagcccagctgctcccatcagcacctcccccaagaacaagaacccacatctgagcccccggtgtctcactgtgtgccgcccacctggcccatccctgcacctccggggtgtctccgctcgcaccagcacgccagccagagagcagaacatgcaagaaggagtgccagcttctcccagggttctgaggccccattcatcatgaggagaaacacagccatgcaaaatcttgcccagcacagtggatacagtgttgatggggtcagcggtcacatccatggcttccatagcctttccaagcccagccagcacaatgcagaattcagaggcagcacccacagaatcccctggagcctggcctcccatggccacaccagaggcagcctcacaggctctgaggcggataatgagggtgtgtaccccttcaaggcacccagaagcaccctgttccaggagtttgggggccacctggtgaacaatagtggtgttccggccaccccactctcagtgcaccagattcctaggacagtcacgttagacaagaacctctatgccatggtggtggccactcctgggcccatagcctcccttcccctgcccaaggcaagtcaggcagaagcatgtcagtggggcagccctcagcagagacctttagtcagtgaaagtagcaggtggtctgttgctgctgccatccccaggcggaatacccttcctgcagtagacaacagccgatgtcaccaagcttcctccggcaagtacacccagcatggtggagggaatgccagccggcctgctgagtccatgcatgagggagtctgttctttcctgccaggaagaacgcttgtgggcctgtcagacagcattgcttctgagggcagctgtgtgcccatgaacccaggctcccctaccctgccggctgtgaagcaagcaggcgatgattcccagggtgtctgcatccctgtgggctcatgtcttgttcgctttgacctgcttggctccccactcacagagctttctatgcaccaagacctcagccagggacatgaggtccagctgccccctgtcaaccgcagcctcaagcctaaccagaaggaccagccaacaccgcccaacctgagaaacaacagggtcatcaatgagctctccttcaagccacctgtcacagagccctggtctgggaccagccacacctttgactccagctcctcccaacaccccatctccacgcagagcatcaccaacacagactcagaagacagtggagagaggtatcttttcccgcagaacccggcatctgcatttcctgtttccggtggcaccagcagttcagccccgccgaggagcactggtaacatccactacgcggccctggacttccagccgagcaagccatccataggctctgtcacgtccggcaagaaggtggactatgtccaggtggatctggagaagacccaggccctgcagaagaccatgcatgaacagatgtgcctgcggcagtcctcagagcctcccaggggcgccaagctgtga

SEQ ID NO: 30 - DNA, 632 bp, Homo sapiens
  misc_feature: Incyte ID No 8186336CB1
cgcctgcgggagcggccggtcggtcgggtccccgcgccccgcacgcccgcacgcccagcggggcccgcattgagcatgggcgcggcggccgtgcgctggcacttgtgcgtgctgctggccctgggcacacgcgggcggctggccgggggcagcgggctcccagggtcagtcgacgtggatgagtgctcagagggcacagatgactgccacatcgatgccatctatcagaacacgcccaagtcctacaaatgcctctgcaagccaggctacaagggggaaggcaagcagtgtgaagatttggtttttctggagacttgatgtaaatagaaccatacgactatgtgatcctgtgacagagacatcactaactctagaagtcaagctgcctggctcccttcctggctccccttgcatttgctgtgtgatctgaggcaggacaggcaatgtctctgagccttggatttgctgctgttgagatagacatggtggtacccagctctcagggcagtcctgtgtgtggggcctgcagactgcctgacatgtcatgagagtcggtcagcaaggccaccgtcgtgattgttcattcatgcaatgcccagaggatgcctttgaacatgctcgggctctgctcg

SEQ ID NO: 31 - DNA, 3044 bp, Homo sapiens
  misc_feature: Incyte ID No 7493330CB1
cgcctcggcctccgtaacccccgcctagccgggccatggcggaacgcggaggggcgggcggtggtcccggaggcgccgggggcggcagcggccagcggggatccggggtcgcccagtcccctcagcagccgccgccgcagcagcagcagcagcagccgccgcagcagccgacgccccccaagctggcccaggccacctcgtcgtcctcgtccacctcggcggcggctgcctcctcctcgtcctcgtctacctccacctccatggccgtggcggtggcctcgggctccgcgcctcccggtggcccggggccaggccgcacccccgccccggtgcagatgaacctgtacgccacctgggaggtggaccggagctcgtccagctgcgtgcctaggctattcagcttgaccctgaagaaactcgtcatgctaaaagaaatggacaaagatcttaactcagtggtcatcgctgtgaagctgcagggttcaaaaagaattcttcgctccaacgagatcgtccttccagctagtggactggtggaaacagagctccaattaaccttctcccttcagtaccctcatttccttaagcgagatgccaacaagctgcagatcatgctgcaaaggagaaaacgttacaagaatcggaccatcttgggctataagaccttggccgtgggactcatcaacatggcagaggtgatgcagcatcctaatgaaggcgcactggtgcttggcctacacagcaacgtgaaggatgtctctgtgcctgtggcagaaataaagatctactccctgtccagccaacccattgaccatgaaggaatcaaatccaagctttctgatcgttctcctgatattgacaattattctgaggaagaggaagagagtttctcatcagaacaggaaggcagtgatgatccattgcatgggcaggacttgttctacgaagacgaagatctccggaaagtgaagaagacccggaggaaactaacctcaacctctgccatcacaaggcaacctaacatcaaacagaagtttgtggccctcctgaagcggtttaaagtttcagatgaggtgggctttgggctggagcatgtgtcccgcgagcagatccgggaagtggaagaggacttggatgaattgtatgacagtctggagatgtacaaccccagcgacagtggccctgagatggaggagacagaaagcatcctcagcacgccaaagcccaagctcaagcctttctttgaggggatgtcgcagtccagctcccagacggagattggcagcctcaacagcaaaggcagcctcggaaaagacaccaccagccctatggaattggctgctctagaaaaaattaaatctacttggattaaaaaccaagatgacagcttgactgaaacagacactctggaaatcactgaccaggacatgtttggagatgccagcacgagtctggttgtgccggagaaagtcaaaactcccatgaagtccagtaaaacggatctccagggctctgcctcccccagcaaagtggagggggtgcacacaccccggcagaagaggagcacgcccctgaaggagcggcagctctccaagcccctaagtgagaggaccaacagttccgacagcgagcgctccccagatctgggccacagcacgcagattccaagaaaggtggtgtatgaccagctcaatcagatcctggtgtcagatgcagccctcccagaaaatgtcattctggtgaacaccactgactggcagggccagtatgtggctgagctgctccaggaccagcggaagcctgtggtgtgcacctgctccaccgtggaggtccaggccgtgctgtccgccctgctcacccggatccagcgctactgcaactgcaactcttccatgccgaggccagtgaaggtggctgctgtgggaggccagagctacctgagctccatcctcaggttctttgtcaagtccctggccaacaagacctccgactggcttggctacatgcgcttcctcatcatccccctcggttctcaccctgtggccaaatacttggggtcagtcgacagtaaatacagtagttccttcctggattctggttggagagatctgttcagtcgctcggagccaccagtgtcagagcaactggacgtggcagggcgggtgatgcagtacgtcaacggggcagccacgacacaccagcttcccgtggccgaagccatgctgacttgccggcataagttccctgatgaagactcctatcagaagtttattcccttcattggcgtggtgaaggtgggtctggttgaagactctccctccacagcaggcgatggggacgattctcctgtggtcagccttactgtgccctccacatcaccaccctccagctcgggcctgagccgagacgccacggccacccctccctcctccccatctatgagcagcgccctggccatcgtggggagccctaatagcccatatggggacgtgattggcctccaggtggactactggctgggccaccccggggagcggaggagggaaggcgacaagagggacgccagctcgaagaacaccctcaagagtgtcttccgctcagtgcaggtgtcccgcctgccccatagtggggaggcccagctttctggcaccatggccatgactgtggtcaccaaagaaaagaacaagaaagttcccaccatcttcctgagcaagaaaccccgagaaaaggaggtggattctaagagccaggtcattgaaggcatcagccgcctcatctgctcagccaagcagcagcagactatgctgagagtgtccatcgatggggtcgagtggagtgacatcaagttcttccagctggcagcccagtggcccacccatgtcaagcactttccagtgggactcttcagtggcagcaaggccacctgaggcctgtctcccagccactttccctcctgcactgccaccagcctcaccgcctgcggcagggggagccacagcccggcccagcaccccttcctgcaaaggacgcttcagcacctgtac

SEQ ID NO: 32 - DNA, 693 bp, Homo sapiens
  misc_feature: Incyte ID No 7487969CB1
ggccaggtccggtgtggggtgtccgagtgccgccggagaggagtggcctcgcccgcttgagtttgattcatcatggataatctgtcatcagaagaaattcaacagagagctcaccagattactgatgagtctctggaaagtacgaggagaatcctgggtttagccattgagtctcaggatgcaggaatcaagaccatcactatgctggatgaacaaaaggaacaactaaaccgcatagaagaaggcttggaccaaataaataaggacatgagagagacagagaagactttaacagaactcaacaaatgctgtggcctttgtgtctgcccatgtaatagcataactaatgatgccagagaagatgaaatggaagagaacctgactcaagtgggcagtatcctgggaaatctaaaagacatggccctgaacataggcaatgagattgatgctcaaaatccacaaataaaacgaatcacagacaaggctgacaccaacagagatcgtattgatattgccaatgccagagcaaagaaactcattgacagtaaagctactgctgttcttctttatcatttattcacttccgtagctcctccttgaaagttattaccttttcagagttaaagttttcggttccacgctcttctaatggggagataatattgggaagaaggggccagagcagttacaagc

SEQ ID NO: 33 - DNA, 3323 bp, Homo sapiens
  misc_feature: Incyte ID No 2655990CB1
ataatgcagagcatgtgaagggagaccggctcggtctctctctctcccagtggactagaaggagcagagagttatgctgtttctcccattctttacagctcaccggatgtaaaagaactctggctagagaccctccaaggacagaggcacagccacacgggagtgaaatccacccctggacagtcagccgcaatactgatgaagctgagaagcagccacaatgcttcaaaaacactaaacgccaataatatggagacactaatcgaatgtcaatcagagggtgatatcaaggaacatcccctgttggcatcatgtgagagtgaagacagtatttgccagctcattgaagttaagaagagaaagaaggtgctgtcctggccctttctcatgagaaggctctcccctgcatcagatttttctggggctttggagacagacttgaaagcatcgctatttgatcagcccttgtcaattatctgcggtgacagtgacacactccccagacccatccaggacattctcactattctatgccttaaaggcccttcaacggaagggatattcaggagagcagccaacgagaaagcccgtaaggagctgaaggaggagctcaactctggggatgcggtggatctggagaggctccccgtgcacctcctcgctgtggtctttaaggacttcctcagaagtatcccccggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtgaagacactggtggaattcctcattgataactgctttgaaatatttggggagaacattccagtgcattccagtatcacttctgatgactccctggagcacactgacagttcagatgtgtcgaccctgcagaatgactcagcctacgacagcaacgaccctgatgtggaatccaacagcagcagtggcatcagctctcccagcaggcagccccaggtgcccatggccacagctgctggcttggatagcgcgggcccacaggatgcccgagaggtcagcccagagcccattgtgagcaccgtggccaggctgaaaagctccctcgcacagcccgataggagatactcagagcccagcatgccatcctcccaggagtgcctcgagagccgggtgacaaaccaaacactaacaaagagtgaaggggacttccccgtgccccgggtaggctctcgtttggaaagtgaggaggctgaagacccatttccagaggaggtcttccctgcagtgcaaggcaaaaccaagaggccggtggacctgaagatcaagaacttggccccgggttcggtgctcccgcgggcactggttctcaaagccttctccagcagctcgctggacgcgtcctctgacagctcgcccgtggcttctccttccagtcccaaaagaaatttcttcagcagacatcagtctttcaccacaaagacagagaaaggcaagcccagccgagaaattaaaaagcactccatgtctttcacctttgcccctcacaaaaaagtgctgaccaaaaacctcagcgcgggctctgggaaatcgcaagactttaccagggaccacgtcccgaggggtgtcagaaaggaaagccagcttgccggccgaatcgtgcaggaaaatgggtgtgaaacccacaaccaaacagcccgcggcttctgcctgagaccccacgccctctcggtggatgatgtgttccagggagctgactgggagaggcctggaagcccaccctcttatgaagaggccatgcagggcccggcagccagactagtggcctccgagagccagaccgtggggagcatgacggtggggagcatgagggcgaggatgctggaggcgcactgcctcctaccccctcttccacctgctcaccacgtagaggactcaagacacaggggcagcaaagagccactccctggccacggactctctcccctgcctgagcgatggaaacagagcagaactgtccatgcttctggggactctctggggcacgtgtctggcccagggagacctgagctcctcccgctgaggaccgtctccgagtccgtgcagaggaataagcgggactgtctcgtgcgacgatgtagccagccggtctttgaggctgaccaattccaatatgccaaagaatcgtatatttaggagggaggccatacgccatgccatagcttgtgctatctgtaaatatgagacttgtaaagaactgcctgtagattgtttttaaaaggtcttgaataagctccttgagaaagttgtggaaagccctcctcagtgaggatagctacaccatggccatggcgcatcagatagtctctgtgtacctggatttgtgcaatatgtaaaaatgtatcaaatgtattatagataaggtgttaggtgcaaaggatgtctaataatccctgcacacgttttgaacttgcagtgaagtacactgctgttccttgcttcctggggcacttttctcttggttagtgtttaaaaattatcttcgcttttttaatgtggcctcaaatgtcatgccaattttcacatcttccacaaactccatttagggagaaatgtttaaatctctggtataagtttactccataccagagtaaactatatattactctatataagcagtcttgcaataactaatcaccaccatagaagaaagaaacagactgcaaggaacagagttgagtgtctggagtcatcaaaggcattaaaaactccagtaaaagctggggccgtagcaaaaatcatgaaaaacacttcaacgtgtcctttcaatcatccaattaaatgtgggtagattaatgaaaatgtattacatcaatattaactcatctatagcactttgagtatctttgtagttcatgatatcctatcctataatgtggaggtaaatgattttatatgcattgggggtcatatataaaacttcaatgtaatttcactacaataaattgccttccttatttgaaagtaaaaatgttctttttttattgacatgttaactcctttccctcaattaatagaaatccactaagagaatcattcacatattggttcactcaacaagcatt

SEQ ID NO: 34 - DNA, 2959 bp, Homo sapiens
  misc_feature: Incyte ID No 71768694CB1
tgatcccctcgcagtgctactgactccaggtgacaggttccggtgtgtcctgggagctcagcctcgctgggggtgaggatccagcatgttgggcactagatggagcttgactagttctggaggcctgggactagtggctgtgggcaagtggttctccccgctgatgctgcccaccagctgctgggccccgggctgctgcggctgggccgcctatggcttgcggtccccctccccatacagccccggcccctggtctctggctgtcagggtttggcctccttcgtggtgaccacctcttcctgtgctcagcgccgggcccaggccccccagcccctgaggacatggtgcatctgcggcggctacaggagatcagtgtggtttctgcagctgacaccccagataagaaagagcatttggtcctggtggagacaggaaggaccctgtatctgcaaggagagggccggctggacttcacggcatggaacgcagccattgggggcgcggctggtgggggcggcacagggctgcaggagcagcagatgagccggggtgacatccccatcatcgtggatgcctgcatcagttttgttacccagcatgggctccggctggaaggtgtataccggaaagggggcgctcgtgcccgcagcctgagactcctggctgagttccgtcgggatgcccggtcggtgaagctccgaccaggggagcactttgtggaggatgtcactgacacactcaaacgcttctttcgtgagctcgatgaccctgtgacctctgcacggttgctgcctcgctggagggaggctgctgagctgccccagaagaatcagcgcctggagaaatataaagatgtgattggctgcctgccgcgggtcaaccgccgcacactggccaccctcattgggcatctctatcgggtgcagaaatgtgcggctctaaaccagatgtgcacgcggaacttggctctgctgtttgcacccagcgtgttccagacggatgggcgaggggagcacgaggtgcgagtgctgcaagagctcattgatggctacatctctgtctttgatatcgattctgaccaggtagctcagattgacttggaggtcagtcttatcaccacctggaaggacgtgcagctgtctcaggctggagacctcatcatggaagtttatatagagcagcagctcccagacaactgtgtcaccctgaaggtgtccccaaccctgactgctgaggagctgactaaccaggtactggagatgcgggggacagcagctgggatggacttgtgggtgacttttgagattcgcgagcatggggagctggagcggccactgcatcccaaggaaaaggtcttagagcaggctttacaatggtgccagctcccagagccctgctcagcctccctgctcttgaaaaaagtccccctggcccaagctggctgcctcttcacaggtatccgacgtgagagcccacgggtggggctgttgcggtgtcgtgaggagccacctcgcttgctgggaagccgcttccaggagaggttctttctgctgcgtggccgctgcctgctgctgctcaaggagaagaaaagctctaaaccagaacgggagtggcctttggaaggtgccaaggtctacctgggaatccgcaagaagttaaagcccccaacaccgtggggcttcacattgatactagagaagatgcacctctacttgtcctgcactgacgaggatgaaatgtgggattggaccaccagcatccttaaagcccagcacgatgaccagcagccagtggtcttacgacgccattcctcctctgaccttgcccgtcagaagtttggcactatgcctttgctgcctatccgtggggatgacagtggagccaccctcctctctgccaatcagaccctgccaatgaagtcatcccaggggtctgtggaggagcaagaggagctggaggagcctgtgtacgaggagccagtgtatgaggaagtaggggccttccctgagttgatccaggacacttctacctccttctccaccacacgggagtggacagtgaagccagagaaccccctcaccagccagaagtcattggatcaaccctttctctccaagtcaagcacccttggccaggaggagaggccacctgagccccctccaggccccccttcaaagagcagtccccaggcacgggggtccctagaggaacagctgctccaggagctcagcagcctcatcctgaggaaaggagagaccactgcaggcctgggaagtccttcccagccatccagcccccaatcccccagccccactggccttccaacacagacacctggcttccccacccaacccccatgcacttccagtccaccctccagccagcccctcacatgaccctaggaccagcagtctgagagggtaggtaccagaagacccagaaactcttatcgtggcactgttgcagcttcctctgccctggctggaaagactccagaatccagtgtggtgctgtggaaggagcactggactaaaggcttcagtggctgcgtgtcccaggacaggtcatggcccctctctgggcccagcccatttatctataccatgaggtaactgaagtaaggagagcagtgaatgtcaaactgtgtttcttagagccataagccccacatattatccctgaacaagggcagctcctgctttatatatttgatacgtaggggttccatgagagattttgggttttaaaggaatggttttactgcattaaagaaaaaaaatgctttggaaaccagaggcctgggtgatgttaaagtctatcctgtcccacttcctacattctgggactaccgtgaagcctggagtagggagagcgagtttgggagctgggactcggggagtcaaaaatagatgagtaattgtcaataaacctgggaaccaaaagacaaaaaaaaaaaaaa

SEQ ID NO: 35 - DNA, 693 bp, Homo sapiens
  misc_feature: Incyte ID No 5079019CB1
gggaagaggcaaagcccatggggagtgctgtgccccatcccttgagccccagctgtgccccttgcagacaaagggttcttcaactgcgatggtttcctggcactaatgggagtttacgagcgcctgtcggctgagcagatcaaggagtacaagggagtctttgagatgttcgacgaagagggcaacggggaggtgaagacgggggagctggagtggctcatgagcctgctgggtatcaaccccaccaagagtgagctggcctcaatggccaaggatgtggacagagacaaggatggggacaggaccatcgactatgagggtgagtggcctatgggagtttaccatgagaaggcccagaaccaggagagcgagctgagggcggcattccgtgtctttgacaaagagggcaagggctacattgactggaacacactcaagtacgtgctaatgaacgcaggggagcccctcaacgaggtggaggcggagcagatgatgaaggaggccgacaaggatggggacaggaccatcgactatgaggagtttgtggccatgatgacgggggagtccttcaagctgatccagtaggtgcagctgccgcagccgggggaggcctgcccgggaaggctgctgcccctgccccctggcccccactcccccggctccgtgtaaaataaatgttccagcccgaaaaaa

SEQ ID NO: 36 - DNA, 4938 bp, Homo sapiens
  misc_feature: Incyte ID No 894500CB1
agaaggggcccacaggcccaaggccatggcgcatggctctcaacctccagctttcagacactgatgacaatgaaacgtttgatgagctgcacatagagagcagtgatgagaaaactccttcagacgtgtcattggctgccgacaccgataagtctgtggagaacctggatgtccttgtggggtttggaaaatctctatgtgggtctcctgaagaggaggaaaaacaagtgcccatcccttcagagactaggccaaagacttttagtttcattaagcagcaaagagttgtaaaaaggacttcttcagaagaatgtgtgactgtgatatttgatgcggaagacggtgagcccattgaattcagctctcaccagactggggttgtcactgttaccagaaatgagatttccatcaattcaactcctgctggacccaaggcagaacatactgagcttttacctcagggaattgcttgtttacagccaagagctgctgcaagagactatactttctttaaaaggtctgaagaggacactgagaaaaacattccaaaagataatgtagataatgttcccagggtgtccactgaatctttcagctccaggacagtgacacaaaatcctcagcagcaaaagctggtcaaaccaacacacaatatatcatgccagagtaattccaggtcttcggcacccatgggcatctatcaaaagcaaaatctgacaaaaatacctcccaggggcaagtcttcacctcagaaatcaaaactaatggagcccgaagccaccacactactcccttcatctggcctggcgactcttgaaaaatcaccagccttagctcctgggaaactctcacgattcatgaagactgagagctcagggccctctttgaattacgatcagatccacacattccaaaacattccgcccaacttccgcacagctccaggatgccccagcaggagggactgggtccagtgccccaagagtcagactccagggtcacggtcaaggcctgccattgagtctagtgacagtggagagccccccacgagggatgaacactgtggctctgggccggaggcaggggtgaaatccccttcccctccgccccctccaggcaggtccgtctccctgctggccaggcccagctatgactattcaccagcaccttcatccaccaagtccgaaaccagggtccccagtgaaacagcaaggaccccattcaaatccccgctgctgaaaggaacttctgctccggttatttcttctaatccggccacgacagaagtgcagaggaagaaaccttctgtggccttcaaaaagcctatcttcactcaccctatgccctccccagaagcagtcattcaaacccgatgccctgctcatgccccctccagctccttcaccgtaatggctctggggcctccaaaggtctctccgaagagaggtgtccccaaaacctctcctcgccaaacacttgggaccccacaaagggacataggattacagactcccaggatctctccttcaacccatgagccactggaaatgacgtcctccaaaagtgtatctccagggagaaaaggacaattgaatgatagcgcctccacaccccccaagccttccttcttaggggtaaatgagtcaccatcatctcaggtcagcagttcctcatcatcctcatcacccgccaaaagccataacagccctcatggttgtcaaagtgctcatgagaaaggactgaaaactcgccttccagtgggactcaaagtgctcatgaagtctccccagctgctcaggaaaagttccaccgtgccagggaaacatgaaaaagacagtttaaatgaagcctccaaaagttccgtggctgtgaacaagtctaagccagaggactccaagaatccagcaagcatggagatcacagcgggtgaaagaaatgtgaccctaccggattcacaagcacagggcagtttagctgatgggcttcccctggaaacagcactacaagagccattggaaagtagcatccctgggagtgatggaagggatggggtagataatagatccatgagaagatccctttcctccagcaaaccacacctaaaaccagctctgggtatgaatggcgccaaagcccgcagccacagcttcagtacacactcaggagacaagccttctacgccccccatcgaagggtcaggcaaagtccgcactcagatcattaccaataccgccgagagaggcaattctcttacccggcagaactcttccacggaaagctctcccaacaaggccccttctgctcccatgttggagagtctccccagtgttgggaggccctcggggcacccctcctccgggaagggctccctggggagctcaggcagcttcagcagtcagcatgggagcccaagtaagttgcctttgaggatccctccaaagtctgagggactcctcatcccacctggaaaggaagaccagcaggccttcacccagggagagtgccccagtgccaatgtggctgtacttggggaaccaggcagtgaccgccgcagttgcccacccaccccaacagactgccctgaagccctgcagagcccagggaggactcagcatccaagcacttttgaaacaagcagtacatccaagctagaaacttctggaaggcatccagatgcctctgcaaccgcgactgatgctgtgagttcagaagcccccctctcacccacaatcgaagaaaaggtcatgttgtgcattcaggaaaatgtggaaaagggccaagtgcaaacaaagcccacctctgtggaagcaaagcagaagcctgggccttcttttgccagctggtttggttttcggaagagtagacttccagctctgagtagcaggaaaatggacatctccaaaaccaaagtagaaaagaaagatgcaaaagtcttggggtttggaaacagacaactaaaatcagaaagaaaaaaagagaaaaagaagcctgaactacagtgtgagacagaaaatgagcttatcaaggacaccaagtcagcagataatccagatggcggtttacaaagcaaaaataatcggagaacaccacaagacatttacaaccaactgaagattgaaccaaggaatagacatagccctgttgcatgttcaacgaaagacaccttcatgacggaactcttgaacagagttgataagaaagcagctccacagacagaaagtggatcaagtaatgcttcctgcaggaatgtgttaaagggcagttctcagggctcctgtctcatcggcagctctatcagtactcaaggaaaccacaagaaaaacatgaaaatcaaagccgatatggaagtaccaaaagactccctggtaaaagaggcaaatgaaaacttgcaagaggatgaagacgatgcagttgcagattctgtatttcagagccacatcatagaatccaactgccagatgagaacattggacagtgggatcggaacctttccactcccagactcgggaaatcgctcgacaggacgctacctatgccagccagactccccagaggacgctgagcctctcctgcctctccagtcagccctttctgcagtttcttccatgagagcccaaacccttgaacgtgaagtgccttcctccacagacggccagcgccctgcagacagcgccattgttcattccacatccgaccccatcatgaccgccagagggatgaggcctcttcagagccgcctccccaaaccagcttcctcaggaaaagtcagttcccaaaagcagaatgaagcagagccaaggcctcagacatgctcatcattcggatatgctgaagacccaatggcaagccagccgcttccagactgggggagtgaagttgctgccaccgggacccaggacaaggcacccagaatgtgtacgtactctgccagcggtggcagtaatagtgacagtgacctggactatggagataatggttttggagctggaaggggacagttagtgaaagcactgaagagcgctgccccagcaggcaagtcctcagagaaggcatgtgcgggggacaacagtgtaaaggtgaaggagcgagcaagccagttgtaggaagtggggaagacctagtgtggtcccaatctccggatgaagggagaaaggggagtctcacctggacccaatggaccttgctggtaaaaagtaaaataacaattctagtaaatccagctctcccaagcttctgtttcatctacctggctgtgtgtcaccaatcaccagccctgccagacaccccaagaggaaacatccaaatggaaacctctcttttttcctctagaaatctgctctgcctcccaggttctctgtcttcactgaatgtctcgccatccttgtaggagtacaaactatgaagcaaggagtcgccctggactctccccctctcatcgtccatgctctggccatcccaaggcatgctgcctcccctcttagaggcttttcaggtctgcctctgtcccactcaccccagcactgcctcggccttggcccccatctgactcacgcagcctgctgcctccctttgtccctagaccctgtcaatttgctgccagtctcgtgccccttaaatgcatcatcctcactgccaccgaagtgccctccctgaaatgccaaagcctgtcatgctgttcctttgctgggaacttttcgagggttgtccatcacccacacagtccactctcctaggtacatgtatctctccaaaatcaccaaggtacacgttaaatatgttcagctttttgtctgtcaattatacctcagtagagtggttttgaaaacaacaacttcaagagcaaggagaggtcccagttgtccttttgctgttaaaataatatttcttgcacaaaaataaagtttaaaatattttaaatgacaaaaaaaaaaaaatttc

SEQ ID NO: 37 - DNA, 2244 bp, Homo sapiens
  misc_feature: Incyte ID No 7497866CB1
tccgcggaaatttgaaatggctgacgggtcgctgacgggcggcggtctggaggcagcggccatggcgccggagcgcacgggctgggcggtggagcaggagctggcgtctctggagaaaggtttgttccaagatgaagattcatgcagtgattgtagctaccgtgataaaccaggttctagtttacaaagttttatgccagaaggaaaaacctttttcccagaaattttccaaacaaatcaacttttgttctatgagcgattcagagcctatcaagattacattttagctgactgcaaggcctctgaggtacaggaattcacagctgagttcttggagaaggtccttgagccatctggatggcgggcagtctggcacactaatgtgttcaaggtgctggttgagatcacagatgtggactttgcagccttgaaggcagtggtgaggcttgctgaaccatacctctgtgactctcaagtgagcacttttaccatggagtgcatgaaggagctccttgatctgaaggagcatcggttgcccctgcaggagctgtgggtggtgtttgatgattcaggagtgtttgaccagacagcccttgcaattgagcatgtcagatttttctaccaaaacatttggaggagttgggatgaagaagaggaggatgaatacgattattttgtcagatgtgttgaacctcgattaagattgcattatgacattcttgaagaccgagttccatcaggacttattgttgactaccacaatctgttgtctcaatgtgaggagagttacaggaaatttttaaatctgagaagcagtttgtcaaattgtaactctgattccgagcaggaaaatatctccatggtggaagggttaaaattgtattcggagatggaacagttgaaacaaaagctgaaactcattgagaatcctttgttgaggtatgtgtttggttatcagaagaattctaacatccaagcaaagggtgtccgttccagcggtcagaagatcactcatgtggtctcctccaccatgatggctggtctcctgcggtccctgcttacggacaggctttgccaggagcctggtgaggaagaaagagaaattcagttccatagtgatccattgtctgctataaatgcctgcttcgaaggtgacactgttattgtttgtcctggccattatgtggtacatggcactttctccattgctgactccattgagttggaaggatatggcctaccagatgacattgtgatagaaaagaggggcaaaggcgacacttttgtggactgcactggtgctgatattaaaatctcaggcataaaatttgttcagcatgatgctgtagagggaatcttaattgttcaccgtggtaagactacgctggaaaactgtgtgctgcagtgtgagacgaccggagtcacagtgcggacatcagcagagtttctaatgaagaactcggatttatatggcgccaagggtgctggtatagaaatctaccctgggagtcagtgcaccctgagtgacaatgggatccatcactgcaaggaagggatcctcattaaggacttcttagatgaacattatgacattcccaagatatccatggtgaataatataatacataataatgaaggttatggtgttgtcttggtgaaacctacaatcttctctgacctgcaagaaaatgctgaagatggaactgaagaaaataaagcgcttaaaattcagacaagtggagagccagatgtggctgaaagagtggatctagaagagctgattgagtgtgcaactggtaaaatggagctttgtgcaagaactgacccttctgagcaagtcgagggaaattgtgaaattgtaaatgaactaattgctgcctccacacagaaaggccagataaagaagaaaaggttgagtgaactggggatcacgcaagctgatgacaacttaatgtcacaggagatgtttgttgggattgtggggaaccagttcaagtggaatgggaaaggtagttttggcacatttcttttctgactacagtgatgtaagtagatagcaaaatactggattttgcacatgctgccctaagaatcactgctgccattgtagtttgctgtattgtctgtattttatatttgattatttgggcttgagtgaaaggtagatttatttccatttgcaggtgttgcacataaaacactccctctttataagaaaaatcataaatgcatataaaatagaca

SEQ ID NO: 38 - DNA, 9353 bp, Homo sapiens
  misc_feature: Incyte ID No 832718CB1
ggctccgaggcgacggccggggggcgggggccgaggcaggtataacggtaccggcggcggcagcgccgctgctcttcccttctcctcaggaggggggccaatggctagcgagaagccgggcccgggcccggggctcgagcctcagcccgtggggctcattgccgtcggggccgctggcggaggcggcgggggcagcggtggtggcggcaccgggggcagcgggatgggggagctaaggggggcgtccggctccggctcggtgatgctccccgcggggatgattaacccttcggtgccgatccgcaacatccggatgaaattcgcagtgttgattggactcatacaggtcggagaggtcagcaacagggacatcgtggagacggtgctcaacctgctggttggtggagaatttgacttggagatgaactttattatccaggatgctgagagtataacatgtatgacagagcttttggagcactgtgatgtaacatgtcaagcagaaatatggagcatgtttacagccattctacgaaaaagtgttcggaatttacagactagcacagaagttgggctaattgaacaagtattgctgaaaatgagtgctgtagatgacatgatagcagatcttctagttgatatgttgggggttcttgccagctacagcatcactgtcaaggagttgaagcttttgttcagcatgcttcgaggagaaagtggaatctggccaagacatgcagtaaaattattatcagttcttaatcagatgccacagagacacggtcctgatacttttttcaatttccctggttgtagcgctgcggcaattgccttgcctcctattgcaaagtggccttatcagaatggcttcaccttaaacacttggtttcgtatggatccattaaataatattaatgttgataaggataaaccttatctttatagttttcgtactagcaaaggagttggttactctgctcattttgttggcaactgtttaatagtcacatcattgaagtccaaaggaaaaggttttcagcattgtgtgaaatatgattttcaaccacgcaagtggtacatgatcagcattgtccacatttacaatcgatggaggaacagtgaaattcggtgttatgttaatggacaactggtatcttatggtgatatggcttggcatgttaacacaaatgatagctatgacaagtgctttcttggatcatcagaaactgctgatgcaaatagggtattctgtggtcaacttggtgccgtgtatgtgttcagtgaagcactcaacccagcacagatatttgcaattcatcagttaggacctggatataagagtaccttcaagtttaaatctgagagtgatattcatttggcagaacatcataaacaggtgttatatgatgggaaacttgcaagtagcattgcctttacatataatgctaaggccactgatgctcagctctgcctggaatcatcaccaaaagagaatgcatcaatttttgtgcattccccacatgctctaatgcttcaggatgtgaaagcgatagtaacacattcaattcatagtgcaattcattcaattggagggattcaagtgctttttccactttttgcccaattggataataggcagctcaatgacagtcaagtggaaacaactgtctgtgctactctgttggcattcctggttgaactacttaaaagttcagtagccatgcaagaacagatgctgggtggaaaaggctttttagtcattggctacttacttgaaaagtcatcaagagttcatataactagagctgtcctggagcaatttttatcttttgcaaaataccttgatggtttatctcatggagcacctttgctgaagcagctttgtgatcacattttatttaacccagccatctggatacatacacctgcaaaggtagttcagctttccctatacacatatttgtctgctgaatttattggaactgctaccatctacaccaccatacgcagagtaggaacagtattacagctaatgcacaccttaaaatattactactgggttattaatcctgctgacagtagtggcattacacctaaaggattagatggtccccggccatcacaaaaagaaattatatcactgagggcatttatgctactttttctgaaacagctgatactaaaggatcgaggggtcaaggaagatgaacttcagagtatattaaattacctacttacgatgcatgaggatgaaaatattcatgatgtgctacagttactggtggctttaatgtcggaacacccagcctcaatgataccagcatttgatcaaagaaatggaataagggtgatctacaaattattggcttctaaaagtgaaagtatttgggttcaagctttgaaggttctgggatactttctgaagcatttaggtcacaagagaaaagttgaaattatgcacacccatagtcttttcactcttcttggagaaaggctgatgttgcatacaaacactgtgactgtcaccacatacaacacactttatgaggtaatcttgacagaacaagtatgtactcaggtcgtacacaaaccacatccagagccagattctacagtgaaaattcagaatccagtgattcttaaagtggtggcaactttgttaaaaaactctacaccaagtgcagagctgatggaagttcgtcgtttatttttatctgatatgataaaacttttcagtaacagccgtgaaaatagaaggtgcttattgcagtgttcagtgtggcaggattggatgttttctcttggctatatcaatcctaaaaattctgaggaacagaagattaccgaaatggtctacaatatcttccggattcttttgtatcatgcaataaaatatgaatggggaggctggagagtctgggtggataccctctcaatagcccattccaaggtaacatatgaagctcataaggaatacctagccaaaatgtatgaggaatatcaaagacaagaggaggaaaacattaaaaagggaaagaaagggaatgtgagcaccatctctggtctttcatcacagacaacaggagcaaaaggtggaatggaaattcgagagatagaagatctttcacaaagccagagcccagaaagtgagaccgattaccctgtcagcacagatactcgagacttactcatgtcaacaaaagtgtcagatgatattcttggaaattcagatagaccaggaagtggtgtacatgtggaagtacatgatcttttagtagatataaaagcagagaaagtggaagcaacagaagtaaagctcgatgatatggatttatcaccggagactttagtaggtggagagaatggtgcccttgtggaggttgaatctctgttggataatgtatatagtgctgctgttgagaaactccagaacaatgtacatggaagtgttggtatcattaaaaaaaatgaagaaaaggataatggtccattgataacattagcagatgagaaagaagaccttcccaatagtagtacatcatttctctttgataaaatacccaaacaggaggaaaaactacttcctgaactttctagcaatcacattattccaaatattcaggacacacaagtacatcttggtgttagtgatgatcttggattgcttgctcacatgaccggtagcgtagacttaacttgtacatccagtataatagaagaaaaagaattcaaaatccatacaacttcagatggaatgagcagtatttctgaaagagacttagcgtcatcaactaaggggctggagtatgctgaaatgactgctacaactctggaaactgagtcttctagtagcaaaattgtaccaaatattgatgcaggaagtataatttcagatactgaaaggtctgacgatggcaaagaatcaggaaaagaaatccgaaaaatccaaacaactactacgacacaagctgtgcagggtcggtctatcacccaacaagaccgagatctccgagttgatttaggatttcgaggaatgccaatgactgaggaacagcgacgccagtttagcccaggtccacggactacaatgtttcgtattcctgagtttaaatggtctccaatgcaccagcggcttctcactgatttactatttgcattagaaactgatgtacatgtttggaggagccattctacaaagtctgtaatggattttgtcaatagcaatgaaaatattatttttgtacataacacaattcacctcatttcccaaatggtagacaacatcatcattgcttgtggaggaattttacctttgctctctgctgctacatcaccaactggttctaagacggaattggaaaatattgaagtgacacaaggcatgtcagctgagacagcagtaactttcctcagccggctgatggctatggttgatgtacttgtgtttgcaagctctctaaattttagtgagattgaagctgagaaaaacatgtcttctggaggtttaatgcgacagtgcctaagattagtttgttgtgttgctgtgagaaactgtttagaatgtcggcaaagacagagagacaggggaaataaatcttcccatggaagcagtaaacctcaggaagttcctcaaagtgtgactgctacagcagcttcgaagactccattggaaaatgttccaggtaacctttctcctattaaggatccggatagacttcttcaggatgttgatatcaatcgccttcgtgctgttgtctttcgggatgtggatgatagcaaacaagcacagttcttagctctggctgttgtttacttcatttcggttctgatggtttccaagtatcgtgacatattagaaccccagagagagactacaagaactggaagccaaccaggtagaaacatcaggcaagaaataaattcaccaacaagtacagaaacacctgctgcatttccagacaccataaaagaaaaagaaacaccaactcctggtgaagatattcaggtagaaagttcaattccccatacagattcaggaattggagaggagcaagtggctagcatcctgaatggggcagaattagaaacaagtacaggccctgatgccatgagtgaactcttatccactttgtcatccgaagtgaagaaatcacaagagagcttaactgaaaatcctagtgaaacgttgaagcctgcaacatccatatctagcattagtcaaaccaaaggcatcaatgtgaaggaaatactgaaaagtcttgtggctgctccagttgaaatagcagaatgtggccctgaacctatcccatacccagatccagcattgaagagagaaacacaagctattcttcctatgcagtttcattcctttgacaggagtgttgtggtgcctgtaaagaaaccacctccaggtagtttagctgtaaccactgtgggagccactactgctggaagtgggctgccaacaggcagtacctctaatatatttgctgctactggagctacaccaaaaagtatgattaatacaacaggtgccgtggattcagggtcctcctcctcttcctcctcttctagttttgtgaatggtgctactagcaaaaaccttccagctgtacaaactgttgctccaatgccagaagattcagctgaaaatatgagcatcactgcaaaacttgaaagagcgttagaaaaagttgctcctcttcttcgtgaaatttttgtagactttgccccattcctatctcgtacacttcttggcagtcatggacaagagctattgatagaaggccttgtttgtatgaagtccagcacatctgtggttgagcttgttatgctgctttgttctcaggaatggcaaaactctattcagaagaatgcaggacttgcatttattgagctcatcaatgaaggaagattactgtgccatgctatgaaggaccatatagtccgtgttgcaaatgaagctgagtttattttgaacagacaaagagccgaggatgtacataaacatgcagagtttgagtcacagtgtgcccaatatgctgctgatagaagagaggaagaaaagatgtgtgaccatcttatcagtgctgctaaacatcgagatcatgtaacagcaaatcagctgaaacagaagattctcaatattctcacaaataaacatggtgcttggggagcagtttctcatagccaattgcatgatttctggcgtttggattactgggaagatgatcttcgtcgaaggagacgatttgttcgcaatgcatttggctccactcatgctgaagcattgctgaaagctgcaatagaatatggcacggaagaagatgtagtaaagtcaaagaaaacattcagaagtcaagcaatagtgaaccaaaatgcagagacagaacttatgctggaaggagacgatgatgcagtcagtctgctacaggagaaagaaattgacaaccttgcaggcccagtggttctcagcacccctgcccagctcatcgctcccgtggtggtggccaaggggactctctccatcaccacgacagaaatctacttcgaggtagatgaggatgattctgccttcaagaagatcgacacgaaagttcttgcatacactgagggacttcacggaaaatggatgttcagcgagatacgagctgtattttcaagacgttaccttctacaaaacactgctttggaagtatttatggcaaaccgaacctcagttatgtttaatttccctgatcaagcaacagtaaaaaaagttgtctatagcttgcctcgggttggagtagggaccagctatggtctgccacaagccaggaggatatcattggccactcctcgacagctttataaatcttccaatatgactcagcgctggcaaagaagggaaatttcaaacttcgaatatttgatgttccttaatactattgcaggacggacatataatgatctgaaccaatatccagtgtttccgtgggtgttaaccaactatgaatcagaagagttggacctgactcttccaggaaacttcagggatctatcaaagccaattggtgctttgaaccccaagagagctgtgttttatgcagagcgttatgagacatgggaagatgatcaaagcccaccctaccattataatacccattattcaacagcaacatctactttatcctggcttgttcgaattgaacctttcacaaccttcttcctcaatgcaaatgatggaaaatttgatcatccagatcgaaccttctcatccgttgcaaggtcttggagaactagtcagagagatacttctgatgtaaaggaactaattccagagttctactacctaccagagatgtttgtcaacagtaatggatataatcttggagtcagagaagatgaagtagtggtaaatgatgttgatcttcccccttgggcaaaaaaacctgaagactttgtgcggatcaacaggatggccctagaaagtgaatttgtttcttgccaacttcatcagtggatcgaccttatatttggctataagcagcgaggaccagaagcagttcgtgctctgaatgtttttcactacttgacttatgaaggctctgtgaacctggatagtatcactgatcctgtgctcagggagattccagaagcttatttcattagagacccccacactttccttcttacaaaggactttattaaggccatggaggcacagatacagaactttggacagacgccatctcagttgcttattgagccacatccgcctcggagctctgccatgcacctgtgtttccttccacagagtccgctcatgtttaaagatcagatgcaacaggatgtgataatggtgctgaagtttccttcaaattctccagtaacccatgtggcagccaacactctgccccacttgaccatccccgcagtggtgacagtgacttgcagccgactctttgcagtgaatagatggcacaacacagtaggcctcagaggagctccaggatactccttggatcaagcccaccatcttcccattgaaatggatccattaatagccaataattcaggtgtaaacaaacggcagatcacagacctcgttgaccagagtatacaaatcaatgcacattgttttgtggtaacagcagataatcgctatattcttatctgtggattctgggataagagcttcagagtttattctacagaaacagggaaattgactcagattgtatttggccattgggatgtggtcacttgcttggccaggtccgagtcatacattggtggggactgctacatcgtgtccggatctcgagatgccaccctgctgctctggtactggagtgggcggcaccatatcataggagacaaccctaacagcagtgactatccggcaccaagagccgtcctcacaggccatgaccatgaagttgtctgtgtttctgtctgtgcagaacttgggcttgttatcagtggtgctaaagagggcccttgccttgtccacaccatcactggagatttgctgagagcccttgaaggaccagaaaactgcttattcccacgcttgatatctgtctccagcgaaggccactgtatcatatactatgaacgagggcgattcagtaatttcagcattaatgggaaacttttggctcaaatggagatcaatgattcaacacgggccattctcctgagcagtgacggccagaacctggtcaccggaggggacaatggggtagtagaggtctggcaggcctgtgacttcaagcaactgtacatttaccctggatgtgatgctggcattagagcaatggacttgtcccatgaccagaggactctgatcactggcatggcttctggtagcattgtagcttttaatatagattttaatcggtggcattatgagcatcagaacagatactgaagataaaggaagaaccaaaagccaagttaaagctgagagcacaagtgctgcatggaaaggcaatatctctggtggaaaaaactcgtctacatcgacctccgtttgtacattccatcacacccagcaatagctgtacattgtagtcagcaaccattttactttgtgtgttttttcacgactgaacaccagctgctatcaagcaagcttatatcatgtaaattatatgaattaggagatgttttggtaattatttcatatattgttgtttattgagaaaaggttgtaggatgtgtcacaagagacttttgacaattctgaggaaccttgtgtccagttgttacaaagtttaagctttgaacctaacctgcatcccatttccagcctcttttcaagctgagaaaaaaaaaaaaaaacacgtttgatactttgtacatcagat

SEQ ID NO: 39 - DNA, 9449 bp, Homo sapiens
  misc_feature: Incyte ID No 7497717CB1
ggctccgaggcgacggccggggggcgggggccgaggcaggtataacggtaccggcggcggcagcgccgctgctcttcccttctcctcaggaggggggccaatggctagcgagaagccgggcccgggcccggggctcgagcctcagcccgtggggctcattgccgtcggggccgctggcggaggcggcgggggcagcggtggtggcggcaccgggggcagcgggatgggggagctaaggggggcgtccggctccggctcggtgatgctccccgcggggatgattaacccttcggtgccgatccgcaacatccggatgaaattcgcagtgttgattggactcatacaggtcggagaggtcagcaacagggacatcgtggagacggtgctcaacctgctggttggtggagaatttgacttggagatgaactttattatccaggatgctgagagtataacatgtatgacagagcttttggagcactgtgatgtaacatgtcaagcagaaatatggagcatgtttacagccattctacgaaaaagtgttcggaatttacagactagcacagaagttgggctaattgaacaagtattgctgaaaatgagtgctgtagatgacatgatagcagatcttctagttgatatgttgggggttcttgccagctacagcatcactgtcaaggagttgaagcttttgttcagcatgcttcgaggagaaagtggaatctggccaagacatgcagtaaaattattatcagttcttaatcagatgccacagagacacggtcctgatacttttttcaatttccctggttgtagcgctgcggcaattgccttgcctcctattgcaaagtggccttatcagaatggcttcaccttaaacacttggtttcgtatggatccattaaataatattaatgttgataaggataaaccttatctttatagttttcgtactagcaaaggagttggttactctgctcattttgttggcaactgtttaatagtcacatcattgaagtccaaaggaaaaggttttcagcattgtgtgaaatatgattttcaaccacgcaagtggtacatgatcagcattgtccacatttacaatcgatggaggaacagtgaaattcggtgttatgttaatggacaactggtatcttatggtgatatggcttggcatgttaacacaaatgatagctatgacaagtgctttcttggatcatcagaaactgctgatgcaaatagggtattctgtggtcaacttggtgccgtgtatgtgttcagtgaagcactcaacccagcacagatatttgcaattcatcagttaggacctggatataagagtaccttcaagtttaaatctgagagtgatattcatttggcagaacatcataaacaggtgttatatgatgggaaacttgcaagtagcattgcctttacatataatgctaaggccactgatgctcagctctgcctggaatcatcaccaaaagagaatgcatcaatttttgtgcattccccacatgctctaatgcttcaggatgtgaaagcgatagtaacacattcaattcatagtgcaattcattcaattggagggattcaagtgctttttccactttttgcccaattggataataggcagctcaatgacagtcaagtggaaacaactgtctgtgctactctgttggcattcctggttgaactacttaaaagttcagtagccatgcaagaacagatgctgggtggaaaaggctttttagtcattggctacttacttgaaaagtcatcaagagttcatataactagagctgtcctggagcaatttttatcttttgcaaaataccttgatggtttatctcatggagcacctttgctgaagcagctttgtgatcacattttatttaacccagccatctggatacatacacctgcaaaggtagttcagctttccctatacacatatttgtctgctgaatttattggaactgctaccatctacaccaccatacgcagagtaggaacagtattacagctaatgcacaccttaaaatattactactgggttattaatcctgctgacagtagtggcattacacctaaaggattagatggtccccggccatcacaaaaagaaattatatcactgagggcatttatgctactttttctgaaacagctgatactaaaggatcgaggggtcaaggaagatgaacttcagagtatattaaattacctacttacgatgcatgaggatgaaaatattcatgatgtgctacagttactggtggctttaatgtcggaacacccagcctcaatgataccagcatttgatcaaagaaatggaataagggtgatctacaaattattggcttctaaaagtgaaagtatttgggttcaagctttgaaggttctgggatactttctgaagcatttaggtcacaagagaaaagttgaaattatgcacacccatagtcttttcactcttcttggagaaaggctgatgttgcatacaaacactgtgactgtcaccacatacaacacactttatgaggtaatcttgacagaacaagtatgtactcaggtcgtacacaaaccacatccagagccagattctacagtgaaaattcagaatccagtgattcttaaagtggtggcaactttgttaaaaaactctacaccaagtgcagagctgatggaagttcgtcgtttatttttatctgatatgataaaacttttcagtaacagccgtgaaaatagaaggtgcttattgcagtgttcagtgtggcaggattggatgttttctcttggctatatcaatcctaaaaattctgaggaacagaagattaccgaaatggtctacaatatcttccggattcttttgtatcatgcaataaaatatgaatggggaggctggagagtctgggtggataccctctcaatagcccattccaaggtaacatatgaagctcataaggaatacctagccaaaatgtatgaggaatatcaaagacaagaggaggaaaacattaaaaagggaaagaaagggaatgtgagcaccatctctggtctttcatcacagacaacaggagcaaaaggtggaatggaaattcgagagatagaagatctttcacaaagccagagcccagaaagtgagaccgattaccctgtcagcacagatactcgagacttactcatgtcaacaaaagtgtcagatgatattcttggaaattcagatagaccaggaagtggtgtacatgtggaagtacatgatcttttagtagatataaaagcagagaaagtggaagcaacagaagtaaagctcgatgatatggatttatcaccggagactttagtaggtggagagaatggtgcccttgtggaggttgaatctctgttggataatgtatatagtgctgctgttgagaaactccagaacaatgtacatggaagtgttggtatcattaaaaaaaatgaagaaaaggataatggtccattgataacattagcagatgagaaagaagaccttcccaatagtagtacatcatttctctttgataaaatacccaaacaggaggaaaaactacttcctgaactttctagcaatcacattattccaaatattcaggacacacaagtacatcttggtgttagtgatgatcttggattgcttgctcacatgaccggtagcgtagacttaacttgtacatccagtataatagaagaaaaagaattcaaaatccatacaacttcagatggaatgagcagtatttctgaaagagacttagcgtcatcaactaaggggctggagtatgctgaaatgactgctacaactctggaaactgagtcttctagtagcaaaattgtaccaaatattgatgcaggaagtataatttcagatactgaaaggtctgacgatggcaaagaatcaggaaaagaaatccgaaaaatccaaacaactactacgacacaagctgtgcagggtcggtctatcacccaacaagaccgagatctccgagttgatttaggatttcgaggaatgccaatgactgaggaacagcgacgccagtttagcccaggtccacggactacaatgtttcgtattcctgagtttaaatggtctccaatgcaccagcggcttctcactgatttactatttgcattagaaactgatgtacatgtttggaggagccattctacaaagtctgtaatggattttgtcaatagcaatgaaaatattatttttgtacataacacaattcacctcatttcccaaatggtagacaacatcatcattgcttgtggaggaattttacctttgctctctgctgctacatcaccaactggttctaagacggaattggaaaatattgaagtgacacaaggcatgtcagctgagacagcagtaactttcctcagccggctgatggctatggttgatgtacttgtgtttgcaagctctctaaattttagtgagattgaagctgagaaaaacatgtcttctggaggtttaatgcgacagtgcctaagattagtttgttgtgttgctgtgagaaactgtttagaatgtcggcaaagacagagagacaggggaaataaatcttcccatggaagcagtaaacctcaggaagttcctcaaagtgtgactgctacagcagcttcgaagactccattggaaaatgttccaggtaacctttctcctattaaggatccggatagacttcttcaggatgttgatatcaatcgccttcgtgctgttgtctttcgggatgtggatgatagcaaacaagcacagttcttagctctggctgttgtttacttcatttcggttctgatggtttccaagtatcgtgacatattagaaccccagagagagactacaagaactggaagccaaccaggtagaaacatcaggcaagaaataaattcaccaacaagtacagttgtggtcataccatctatccctcatccaagtttgaaccatggattccttgccaagttaattcctgagcagagctttggccactcattttacaaagaaacacctgctgcatttccagacaccataaaagaaaaagaaacaccaactcctggtgaagatattcaggtagaaagttcaattccccatacagattcaggaattggagaggagcaagtggctagcatcctgaatggggcagaattagaaacaagtacaggccctgatgccatgagtgaactcttatccactttgtcatccgaagtgaagaaatcacaagagagcttaactgaaaatcctagtgaaacgttgaagcctgcaacatccatatctagcattagtcaaaccaaaggcatcaatgtgaaggaaatactgaaaagtcttgtggctgctccagttgaaatagcagaatgtggccctgaacctatcccatacccagatccagcattgaagagagaaacacaagctattcttcctatgcagtttcattcctttgacaggagtgttgtggtgcctgtaaagaaaccacctccaggtagtttagctgtaaccactgtgggagccactactgctggaagtgggctgccaacaggcagtacctctaatatatttgctgctactggagctacaccaaaaagtatgattaatacaacaggtgccgtggattcagggtcctcctcctcttcctcctcttctagttttgtgaatggtgctactagcaaaaaccttccagctgtacaaactgttgctccaatgccagaagattcagctgaaaatatgagcatcactgcaaaacttgaaagagcgttagaaaaagttgctcctcttcttcgtgaaatttttgtagactttgccccattcctatctcgtacacttcttggcagtcatggacaagagctattgatagaaggccttgtttgtatgaagtccagcacatctgtggttgagcttgttatgctgctttgttctcaggaatggcaaaactctattcagaagaatgcaggacttgcatttattgagctcatcaatgaaggaagattactgtgccatgctatgaaggaccatatagtccgtgttgcaaatgaagctgagtttattttgaacagacaaagagccgaggatgtacataaacatgcagagtttgagtcacagtgtgcccaatatgctgctgatagaagagaggaagaaaagatgtgtgaccatcttatcagtgctgctaaacatcgagatcatgtaacagcaaatcagctgaaacagaagattctcaatattctcacaaataaacatggtgcttggggagcagtttctcatagccaattgcatgatttctggcgtttggattactgggaagatgatcttcgtcgaaggagacgatttgttcgcaatgcatttggctccactcatgctgaagcattgctgaaagctgcaatagaatatggcacggaagaagatgtagtaaagtcaaagaaaacattcagaagtcaagcaatagtgaaccaaaatgcagagacagaacttatgctggaaggagacgatgatgcagtcagtctgctacaggagaaagaaattgacaaccttgcaggcccagtggttctcagcacccctgcccagctcatcgctcccgtggtggtggccaaggggactctctccatcaccacgacagaaatctacttcgaggtagatgaggatgattctgccttcaagaagatcgacacgaaagttcttgcatacactgagggacttcacggaaaatggatgttcagcgagatacgagctgtattttcaagacgttaccttctacaaaacactgctttggaagtatttatggcaaaccgaacctcagttatgtttaatttccctgatcaagcaacagtaaaaaaagttgtctatagcttgcctcgggttggagtagggaccagctatggtctgccacaagccaggaggatatcattggccactcctcgacagctttataaatcttccaatatgactcagcgctggcaaagaagggaaatttcaaacttcgaatatttgatgttccttaatactattgcaggacggacatataatgatctgaaccaatatccagtgtttccgtgggtgttaaccaactatgaatcagaagagttggacctgactcttccaggaaacttcagggatctatcaaagccaattggtgctttgaaccccaagagagctgtgttttatgcagagcgttatgagacatgggaagatgatcaaagcccaccctaccattataatacccattattcaacagcaacatctactttatcctggcttgttcgaattgaacctttcacaaccttcttcctcaatgcaaatgatggaaaatttgatcatccagatcgaaccttctcatccgttgcaaggtcttggagaactagtcagagagatacttctgatgtaaaggaactaattccagagttctactacctaccagagatgtttgtcaacagtaatggatataatcttggagtcagagaagatgaagtagtggtaaatgatgttgatcttcccccttgggcaaaaaaacctgaagactttgtgcggatcaacaggatggccctagaaagtgaatttgtttcttgccaacttcatcagtggatcgaccttatatttggctataagcagcgaggaccagaagcagttcgtgctctgaatgtttttcactacttgacttatgaaggctctgtgaacctggatagtatcactgatcctgtgctcagggagattccagaagcttatttcattagagacccccacactttccttcttacaaaggactttattaaggccatggaggcacagatacagaactttggacagacgccatctcagttgcttattgagccacatccgcctcggagctctgccatgcacctgtgtttccttccacagagtccgctcatgtttaaagatcagatgcaacaggatgtgataatggtgctgaagtttccttcaaattctccagtaacccatgtggcagccaacactctgccccacttgaccatccccgcagtggtgacagtgacttgcagccgactctttgcagtgaatagatggcacaacacagtaggcctcagaggagctccaggatactccttggatcaagcccaccatcttcccattgaaatggatccattaatagccaataattcaggtgtaaacaaacggcagatcacagacctcgttgaccagagtatacaaatcaatgcacattgttttgtggtaacagcagataatcgctatattcttatctgtggattctgggataagagcttcagagtttattctacagaaacagggaaattgactcagattgtatttggccattgggatgtggtcacttgcttggccaggtccgagtcatacattggtggggactgctacatcgtgtccggatctcgagatgccaccctgctgctctggtactggagtgggcggcaccatatcataggagacaaccctaacagcagtgactatccggcaccaagagccgtcctcacaggccatgaccatgaagttgtctgtgtttctgtctgtgcagaacttgggcttgttatcagtggtgctaaagagggcccttgccttgtccacaccatcactggagatttgctgagagcccttgaaggaccagaaaactgcttattcccacgcttgatatctgtctccagcgaaggccactgtatcatatactatgaacgagggcgattcagtaatttcagcattaatgggaaacttttggctcaaatggagatcaatgattcaacacgggccattctcctgagcagtgacggccagaacctggtcaccggaggggacaatggggtagtagaggtctggcaggcctgtgacttcaagcaactgtacatttaccctggatgtgatgctggcattagagcaatggacttgtcccatgaccagaggactctgatcactggcatggcttctggtagcattgtagcttttaatatagattttaatcggtggcattatgagcatcagaacagatactgaagataaaggaagaaccaaaagccaagttaaagctgagagcacaagtgctgcatggaaaggcaatatctctggtggaaaaaactcgtctacatcgacctccgtttgtacattccatcacacccagcaatagctgtacattgtagtcagcaaccattttactttgtgtgttttttcacgactgaacaccagctgctatcaagcaagcttatatcatgtaaattatatgaattaggagatgttttggtaattatttcatatattgttgtttattgagaaaaggttgtaggatgtgtcacaagagacttttgacaattctgaggaaccttgtgtccagttgttacaaagtttaagctttgaacctaacctgcatcccatttccagcctcttttcaagctgagaaaaaaaaaaaaaaacacgtttgatactttgtacatcagat

SEQ ID NO: 40 - DNA, 2065 bp, Homo sapiens
  misc_feature: Incyte ID No 7506420CB1
ttgaaatggctgacgggtcgctgacgggcggcggtctggaggcagcggccatggcgccggagcgcacgggctgggcggtggagcaggagctggcgtctctggagaaagctgactgcaaggcctctgaggtacaggaattcacagctgagttcttggagaaggtccttgagccatctggatggcgggcagtctggcacactaatgtgttcaaggtgctggttgagatcacagatgtggactttgcagccttgaaggcagtggtgaggcttgctgaaccatacctctgtgactctcaagtgagcacttttaccatggagtgcatgaaggagctccttgatctgaaggagcatcggttgcccctgcaggagctgtgggtggtgtttgatgattcaggagtgtttgaccagacagcccttgcaattgagcatgtcagatttttctaccaaaacatttggaggagttgggatgaagaagaggaggatgaatacgattattttgtcagatgtgttgaacctcgattaagattgcattatgacattcttgaagaccgagttccatcaggacttattgttgactaccacaatctgttgtctcaatgtgaggagagttacaggaaatttttaaatctgagaagcagtttgtcaaattgtaactctgattccgagcaggaaaatatctccatggtggaagggttaaaattgtattcggagatggaacagttgaaacaaaagctgaaactcattgagaatcctttgttgaggtatgtgtttggttatcagaagaattctaacatccaagcaaagggtgtccgttccagcggtcagaagatcactcatgtggtctcctccaccatgatggctggtctcctgcggtccctgcttacggacaggctttgccaggagcctggtgaggaagaaagagaaattcagttccatagtgatccattgtctgctataaatgcctgcttcgaaggtgacactgttattgtttgtcctggccattatgtggtacatggcactttctccattgctgactccattgagttggaaggatatggcctaccagatgacattgtgatagaaaagaggggcaaaggcgacacttttgtggactgcactggtgctgatattaaaatctcaggcataaaatttgttcagcatgatgctgtagagggaatcttaattgttcaccgtggtaagactacgctggaaaactgtgtgctgcagtgtgagacgaccggagtcacagtgcggacatcagcagagtttctaatgaagaactcggatttatatggcgccaagggtgctggtatagaaatctaccctgggagtcagtgcaccctgagtgacaatgggatccatcactgcaaggaagggatcctcattaaggacttcttagatgaacattatgacattcccaagatatccatggtgaataatataatacataataatgaaggttatggtgttgtcttggtgaaacctacaatcttctctgacctgcaagaaaatgctgaagatggaactgaagaaaataaagcgcttaaaattcagacaagtggagagccagatgtggctgaaagagtggatctagaagagctgattgagtgtgcaactggtaaaatggagctttgtgcaagaactgacccttctgagcaagtcgagggaaattgtgaaattgtaaatgaactaattgctgcctccacacagaaaggccagataaagaagaaaaggttgagtgaactggggatcacgcaagctgatgacaacttaatgtcacaggagatgtttgttgggattgtggggaaccagttcaagtggaatgggaaaggtagttttggcacatttcttttctgactacagtgatgtaagtagatagcaaaatactggattttgcacatgctgccctaagaatcactgctgccattgtagtttgctgtattgtctgtattttatatttgattatttgggcttgagtgaaaggtagatttatttccatttgcaggtgttgcacataaaacactccctctttataagaaaaatcataaatgcatataaaatagaca